(19) United States
(12) Patent Application Publication
(10) Pub. No.: US 2003/0068671 A1
(43) Pub. Date: Apr. 10, 2003

(54) Novel proteins and nucleic acids encoding same

(76) Inventors: Muralidhara Padigaru, Bronx, NY (US); Catherine E. Burgess, Wethersfield, CT (US); Vishnu Mishra, Gainesville, FL (US); Li Li, Cheshire, CT (US); Jason C. Baumgartner, Milford, CT (US); Kumud Majumder, Stamford, CT (US); Kimberly A. Spytek, New Haven, CT (US); Velizar T. Tchernev, Branford, CT (US)

Correspondence Address: Ivor R. Elrifi, MINTZ, LEVIN, COHN, FERRIS,, GLOVSKY and POPEO, P.C., One Financial Center, Boston, MA, 02111, US

(21) Appl. No.: 09/800,321

(22) Filed: Mar. 5, 2001

Related U.S. Application Data
(60) Provisional application No. 60/186,606, filed on Mar. 3, 2000.
(60) Provisional application No. 60/221,942, filed on Jul. 31, 2000.
(60) Provisional application No. 60/260,285, filed on Jan. 8, 2001.
(60) Provisional application No. 60/220,263, filed on Jul. 24, 2000.
(60) Provisional application No. 60/257,600, filed on Dec. 21, 2000.
(60) Provisional application No. 60/187,295, filed on Mar. 6, 2000.
(60) Provisional application No. 60/187,247, filed on Mar. 6, 2000.
(60) Provisional application No. 60/219,854, filed on Jul. 21, 2000.
(60) Provisional application No. 60/187,250, filed on Mar. 6, 2000.
(60) Provisional application No. 60/187,249, filed on Mar. 6, 2000.
(60) Provisional application No. 60/187,253, filed on Mar. 6, 2000.
(60) Provisional application No. 60/187,248, filed on Mar. 6, 2000.
(60) Provisional application No. 60/187,296, filed on Mar. 6, 2000.
(60) Provisional application No. 60/187,563, filed on Mar. 7, 2000.

(51) Int. Cl. C07K 14/705; C12Q 1/68; C07H 21/04; C12P 21/02; C12N 5/06

(52) U.S. Cl. 435/69.1; 435/320.1; 435/325; 530/350; 536/23.5; 435/6

(57) ABSTRACT

Disclosed herein are nucleic acid sequences that encode G-coupled protein-receptor related polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority from Provisional Applications U.S. Ser. No. 60/186,606, filed Mar. 3, 2000; U.S. Ser. No. 60/221,942, filed Jul. 31, 2000; U.S. Ser. No. 60/260,285, filed Jan. 8, 2001; U.S. Ser. No. 60/220,263, filed Jul. 24, 2000; U.S. Ser. No. 60/257,600, filed Dec. 21, 2000; U.S. Ser. No. 60/187,295, filed Mar. 6, 2000; U.S. Ser. No. 60/187,247, filed Mar. 6, 2000; U.S. Ser. No. 60/219,854, filed Jul. 21, 2000; U.S. Ser. No. 60/187,250, filed Mar. 6, 2000; U.S. Ser. No. 60/187,249, filed Mar. 6, 2000; U.S. Ser. No. 60/187,253, filed Mar. 6, 2000; U.S. Ser. No. 60/187, 248, filed Mar. 6, 2000; U.S. Ser. No. 60/187,296, filed Mar. 6, 2000; and U.S. Ser. No. 60/187,563, filed Mar. 7, 2000, each of which is incorporated by reference in its entirety.

### BACKGROUND OF THE INVENTION

The invention generally relates to nucleic acids and polypeptides. More particularly, the invention relates to nucleic acids encoding novel G-protein coupled receptor (GPCR) polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, NOV8, NOV9, and NOV10 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS:1, 3, 5, 7, 9, 11, 13, 15, 17, 19, 20, 22, 24, 26, 28, and 30. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS:2, 4, 6, 8, 10, 12, 14, 16, 18, 21, 23, 25, that encodes a NOVX polypeptide, or a fragment, homolog, analog or derivative thereof. For example, the nucleic acid can encode a polypeptide at least 80% identical to a polypeptide comprising the amino acid sequences of SEQ ID NOS:2, 4, 6, 8, 10, 12, 14, 16, 18, 21, 23, 25, 27, 29, and 31. The nucleic acid can be, for example, a genomic DNA fragment or a cDNA molecule that includes the nucleic acid sequence of any of SEQ ID NOS:1, 3, 5, 7, 9, 11, 13, 15, 17, 19, 20, 22, 24, 26, 28, and 30.

Also included in the invention is an oligonucleotide, e.g., an oligonucleotide which includes at least 6 contiguous nucleotides of a NOVX nucleic acid (e.g., SEQ ID NOS:1, 3, 5, 7, 9, 11, 13, 15, 17, 19, 20, 22, 24, 26, 28, and 30) or a complement of said oligonucleotide.

Also included in the invention are substantially purified NOVX polypeptides (SEQ ID NOS:2, 4, 6, 8, 10, 12, 14, 16, 18, 21, 23, 25, 27, 29, and 31). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, or other disorders related to cell signal processing and metabolic pathway modulation. The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from: developmental diseases, MHCII and III diseases (immune diseases), taste and scent detectability Disorders, Burkitt's lymphoma, corticoneurogenic disease, signal transduction pathway disorders, Retinal diseases including those involving photoreception, Cell growth rate disorders; cell shape disorders, feeding disorders; control of feeding; potential obesity due to over-eating; potential disorders due to starvation (lack of appetite), noninsulin-dependent diabetes mellitus (NIDDM1), bacterial, fungal, protozoal and viral infections (particularly infections caused by HIV-1 or HIV-2), pain, cancer (including but not limited to neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation, dentatorubro-pallidoluysian atrophy (DRPLA) hypophosphatemic rickets, autosomal dominant (2) acrocallosal syndrome and dyskinesias, such as Huntington's disease or Gilles de la Tourette syndrome and/or other pathologies and disorders of the like.

The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of nonlimiting example, the compositions of the present invention will have efficacy for treatment of patients suffering from bacterial, fungal, protozoal and viral infections (particularly infections caused by HIV-1 or HIV-2), pain, cancer (including but not limited to neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease, multiple sclerosis, and treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders.

The invention further includes a method for screening for a modulator of disorders or syndromes including, e.g., diabetes, metabolic disturbances associated with obesity, the metabolic syndrome X, anorexia, wasting disorders associated with chronic diseases, metabolic f disorders, diabetes, obesity, infectious disease, anorexia, cancer-associated cachexia, cancer, neurodegenerative disorders, Alzheimer's Disease, Parkinson's Disorder, immune disorders, and hematopoietic disorders or other disorders related to cell signal processing and metabolic pathway modulation. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to an disorders or syndromes including, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders or other disorders related to cell signal processing and metabolic pathway modulation by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders. Also, the expression levels of the new polypeptides of the invention can be used in a method to screen for various cancers as well as to determine the stage of cancers.

In a further aspect, the invention includes a method of treating or preventing a q pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders.

In yet another aspect, the invention can be used in a method to identify the cellular receptors and downstream effectors of the invention by any one of a number of techniques commonly employed in the art. These include but are not limited to the two-hybrid system, affinity purification, co-precipitation with antibodies or other specific-interacting molecules.

In another aspect, the invention is concerned with a method of screening a candidate substance interacting with an olfactory receptor polypeptide of SEQ ID NOS:2, 4, 6, 8, 10, 12, 14, 16, 18, 21, 23, 25, 27, 29 or 31, or fragments thereof. In this aspect, the method involves providing an olfactory receptor polypeptide; obtaining a candidate substance; contacting the polypeptide and the candidate substance; and detecting the complexes formed, if any.

In a further aspect, the invention is concerned with a method of screening ligand molecules interacting with an olfactory receptor polypeptide according to the invention. In this aspect, the method involves providing a recombinant eukaryotic host cell containing a nucleic acid molecule encoding the polypeptide; preparing membrane extracts of the recombinatnt eukaryotic host cell; contacting the membrane extracts with a selected ligand molecule; and detecting the production olevel of second messenger metabolites.

In a still further aspect, the method of screening ligand molecules comprises the steps of providing an adenovirus containing a nucleic acid encoding an olfactory receptor polypeptide; infecting an olfactory epithelium with the adenovirus; contacting the olactory epithelium with a selected ligand molecule; and detecting an increase, if any, of the response to the ligand molecule.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The invention is based, in part, upon the discovery of novel nucleic acid sequences that encode novel polypeptides. The novel nucleic acids and their encoded polypeptides are referred to individually as NOV1, NOV2, NOV3, NOV4, NOV5, NOV6, NOV7, NOV8, NOV9, and NOV10. The nucleic acids, and their encoded polypeptides, are collectively designated herein as “NOVX”.

The novel NOVX nucleic acids of the invention include the nucleic acids whose sequences are provided in Tables 1A, 1C, 1E, 2A, 2C, 2E, 2G, 3A, 4A, 4C, 5A, 6A, 7A, 8A, 9A, and 10A, inclusive, or a fragment, derivative, analog or homolog thereof. The novel NOVX proteins of the invention include the protein fragments whose sequences are provided in Tables 1B, 1D, 1F, 2B, 2D, 1F, 2H, 3B, 4B, 5B, 6B, 7B, 8B, 9B, and 10B, inclusive. The individual NOVX nucleic acids and proteins are described below. Within the scope of this invention is a method of using these nucleic acids and peptides in the treatment or prevention of a disorder related to cell signaling or metabolic pathway modulation.

G-Protein Coupled Receptor proteins (GPCRs) have been identified as a large family of G protein-coupled receptors in a number of species. These receptors share a seven transmembrane domain structure with many neurotransmitter and hormone receptors, and are likely to underlie the recognition and G-protein-mediated transduction of various signals. Human GPCR generally do not contain introns and belong to four different gene subfamilies, displaying great sequence variability. These genes are dominantly expressed in olfactory epithelium. See, e.g., Ben-Arie et al., Hum. Mol. Genet. 1994 3:229-235; and, Online Mendelian Inheritance in Man (OMIM) entry #164342 (http://www.ncbi.nlm.nih.gov/entrez/dispomim.cgi?).

The olfactory receptor (OR) gene family constitutes one of the largest GPCR multigene families and is distributed among many chromosomal sites in the human genome. See Rouquier et al., Hum. Mol. Genet. 7(9):1337-45 (1998); Malnic et al., Cell 96:713-23 (1999). Olfactory receptors constitute the largest family among G protein-coupled receptors, with up to 1000 members expected. See Vanderhaeghen et al., Genomics 39(3):239-46 (1997); Xie et al., Mamm. Genome 11(12):1070-78 (2000); Issel-Tarver et al., Proc. Natl. Acad. Sci. USA 93(20):10897-902 (1996). The recognition of odorants by olfactory receptors is the first stage in odor discrimination. See Krautwurst et al., Cell 95(7):917-26 (1998); Buck et al., Cell 65(1):175-87 (1991). Many ORs share some characteristic sequence motifs and have a central variable region corresponding to a putative ligand binding site. See Issel-Tarver et al., Proc. Natl. Acad. Sci. USA 93:10897-902 (1996).

Other examples of seven membrane spanning proteins that are related to GPCRs are chemoreceptors. See Thomas et al., Gene 178(1-2):1-5 (1996). Chemoreceptors have been identified in taste, olfactory, and male reproductive tissues. See id; Walensky et al., J. Biol. Chem. 273(16):9378-87 (1998); Parmentier et al., Nature 355(6359):453-55 (1992); Asai et al., Biochem. Biophys. Res. Commun. 221(2):240-47 (1996).

NOV1 includes a family of three novel G-protein coupled receptor (“GPCR”) proteins disclosed below. The disclosed proteins have been named NOV1a, NOV1b, and NOV1c and are each related to olfactory receptors.

NOV1a

A novel NOV1a nucleic acid was identified on chromosome 6 by ThlastN using CuraGen Corporation's sequence file for GPCR probes or homologs, and run against the Genomic Daily Files made available by GenBank. The nucleic acid was further predicted by the program GenScan™, including selection of exons. These were further modified by means of similarities using BLAST searches. The sequences were then manually corrected for apparent inconsistencies, thereby obtaining the sequences encoding the fill-length protein.

A disclosed NOV1a nucleic acid of 993 (designated CuraGen Acc. No. dj408b20_B) nucleotides is shown in Table 1A. The disclosed NOV1a open reading frame (“ORF”) begins at the ATG initiation codon at nucleotides 15-17, shown in bold in Table 1A. The encoded polypeptide is referred to herein as NOV1a polypeptide. The disclosed NOV1a ORF terminates at a TAA codon at nucleotides 954-956. As shown in Table 1A, putative untranslated regions 5′ to the start codon and 3′ to the stop codon are underlined, and the start and stop codons are in bold letters.

TABLE 1A
NOV1a nucleotide sequence (SEQ ID NO:1).

TTGAAACATTAATCATGAATTGGGTAAATGACAGCATCATACAGGAGTTT
ATTCTGCTGGGTTTCTCAGATCGACCTTGGCTGGAGTTTCCACTCCTTGT
GGTCTTCTTGATTTCTTACACTGTGACCATCTTTGGCAATCTGACCATTA
TTCTAGTGTCACGCCTGGACACCAAACTTCATACCCCCATGTATTTTTTT
CTTACCAATCTATCACTCCTGGATCTTTGTTACACCACATGTACAGTCCC
ACAAATGCTAGTAAATTTATGCAGCATCAGGAAAGTAATCAGTTATCGTG
GCTGTGTAGCCCAGCTTTTCATATTTCTGGCCTTGGGGGCTACTGAATAT
CTTCTCCTGGCCGTCATGTCCTTTGATAGGTTTGTAGCTATTTGTCGGCC
TCTCCATTACTCAGTTATCATGCACCAGAGACTCTGCCTCCAGTTGGCAG
CTGCATCCTGGGTTACTGGTTTTAGTAACTCAGTGTGGTTGTCTACCCTG
ACTCTCCAGCTGCCACTCTGTGACCCCTATGTGATAGATCACTTTCTCTG
TGAAGTCCCTGCACTGCTCAAGTTATCTTGTGTTGAGACAACAGCAAATG
AGGCTGAACTATTCCTTGTCAGTGAGCTCTTCCATCTAATACCCCTGACA
CTCATCCTTATATCATATGCTTTTATTGTCCGAGCAGTATTGAGGATACA
GTCTGCTGAAGGTCGACAAAAAGCATTTGGGACATGTGGTTCCCATCTAA
TTGTGGTGTCTCTTTTTTATAGTACAGCCGTCTCTGTGTACCTGCAACCA
CCTTCGCCCAGCTCCAAGGACCAAGGAAAGATGGTTTCTCTCTTCTATGG
AATCATTGCACCCATGCTGAATCCCCTTATATATACACTTAGGAACAAGG
AGGTAAAGGAAGGCTTTAAAAGGTTGGTTGCAAGAGTCTTCTTAATCAAG
AAATAAGAAATATGCAAATGATAAGTTTGCTAAAGACAAAAT

A disclosed encoded NOV1a protein has 313 amino acid residues and is referred to as the NOV1a protein. The NOV1a protein was analyzed for signal peptide prediction and cellular localization. SignalP results predict that NOV1a is cleaved between position 38 and 39 of SEQ ID NO:2, i.e., at the slash in the amino acid sequence TVT-IF. Psort and Hydropathy profiles also predict that NOV1a contains a signal peptide and is likely to be localized at the plasma membrane (certainty of 0.6000), Golgi body (certainty of 0.400), endoplasmic reticulum (membrane) (certainty of 0.3000), and microbody (peroxisome) (certainity of 0.3000). The disclosed NOV1a polypeptide sequence is presented in Table 1B using the one-letter amino acid code.

TABLE 1B
Encoded NOV1a protein sequence (SEQ ID NO:2).

MNWVNDSIIQEFILLGFSDRPWLEFPLLVVFLISYTVTIFGNLTIILVSR
LDTKLHTPMYFFLTNLSLLDLCYTTCTVPQMLVNLCSIRKVISYRGCVAQ
LFIFLALGATEYLLLAVMSFDRFVAICRPLHYSVIMHQRLCLQLAAASWV
TGFSNSVWLSTLTLQLPLCDPYVIDHFLCEVPALLKLSCVETTANEAELF
LVSELFHLIPLTLILISYAFIVRAVLRIQSAEGRQKAFGTCGSHLIVVSL
FYSTAVSVYLQPPSPSSKDQGKMVSLFYGIIAPMLNPLIYTLRNKEVKEG
FKRLVARVFLIKK

NOV1b

A target sequence identified previously, Accession Number dj408b20_B (NOV1a), was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such suitable sequences were then employed as the forward and reverse primers in a PCR amplification based on a library containing a wide range of cDNA species. The resulting amplicon was gel purified, cloned and sequenced to high redundancy to provide the sequence reported below, which is designated NOV1b (Accession Number dj408b20B-1). There is one nucleotide change at position 365 of the sequence of dj408b20B-1.

A disclosed NOV1b nucleic acid of 943 nucleotides is shown in Table 1C. An open reading frame was identified beginning with an ATG initiation codon at nucleotides 2-4 and ending with a AAA codon at nucleotides 938-940. A putative untranslated region upstream from the initiation codon and downstream from the termination codon is underlined in Table 1C, and the start and stop codons are in bold letters. NOV1b includes a Kozak sequence.

TABLE 1C
NOV1b Nucleic acid sequence (SEQ ID NO:3).

TATGAATTGGGTAAATGACAGCATCATACAGGAGTTTATTCTGCTGGGTTTCTCAGATCGACCTTGGCTGGAGT
TTCCACTCCTTGTGGTCTTCTTGATTTCTTACACTGTGACCATCTTTGGCAATCTGACCATTATTCTAGTGTCA
CGCCTGGACACCAAACTTCATACGCCCATGTATTTTTTTCTTACCAATCTATCACTCCTGGATCTTTGTTACAC
CACATGTACAGTCCCACAAATGCTAGTAAATTTATGCAGCATCAGGAAAGTAATCAGTTATCGTGGCTGTGTAG
CCCAGCTTTTCATATTTCTGGCCTTGGGGGCTACTGAATATCTTCTCCTGGCCGTCATGTCCTTTGATTGGTTT
GTAGCTATTTGTCGGCCTCTCCATTACTCAGTTATCATGCACCAGAGACTCTGCCTCCAGTTGGCAGCTGCATC
CTGGGTTACTGGTTTTAGTAACTCAGTGTGGTTGTCTACCCTGACTCTCCAGCTGCCACTCTGTGACCCCTATG
TGATAGATCACTTTCTCTGTGAAGTCCCTGCACTGCTCAAGTTATCTTGTGTTGAGACAACAGCAAATGAGGCT
GAACTATTCCTTGTCAGTGAGCTCTTCCATCTAATACCCCTGACACTCATCCTTATATCATATGCTTTTATTGT
CCGAGCAGTATTGAGGATACAGTCTGCTGAAGGTCGACAAAAAGCATTTGGGACATGTGGTTCCCATCTAATTG
TGGTGTCTCTTTTTTATAGTACAGCCGTCTCTGTGTACCTGCAACCACCTTCGCCCAGCTCCAAGGACCAAGGA
AAGATGGTTTCTCTCTTCTATGGAATCATTGCACCCATGCTGAATCCCCTTATATATACACTTAGGAACAAGGA
GGTAAAGGAAGGCTTTAAAAGGTTGGTTGCAAGAGTCTTCTTAATCAAGAAATAA

A disclosed encoded NOV1b protein has 313 amino acid residues (SEQ ID NO:4) and is referred to as the NOV1b protein. The NOV1b protein was analyzed for signal peptide prediction and cellular localization. SignalP results predict that NOV1b is cleaved between position 38 and 39 of SEQ ID NO:4, i.e., at the slash in the amino acid sequence TVT-IF. Psort and Hydropathy profiles also predict that NOV1b contains a signal peptide and is likely to be localized at the plasma membrane (certainty of 0.6000). The disclosed NOV1b polypeptide sequence is presented in Table 1D using the one-letter amino acid code.

TABLE 1D
Encoded NOV1b protein sequence (SEQ ID NO:4).

MNWVNDSIIQEFILLGFSDRPWLEFPLLVVFLISYTVTIFGNLTIILVSRLDTKLHTPMYFFLTNLSLLD
LCYTTCTVPQMLVNLCSIRKVISYRGCVAQLFIFLALGATEYLLLAVMSFDWFVAICRPLHYSVIMHQRLCLQLAAA
SWVTGFSNSVWLSTLTLQLPLCDPYVIDHFLCEVPALLKLSCVETTANEAELFLVSELFHLIPLTLILISYAFIVRA
VLRIQSAEGRQKAFGTCGSHLIVVSLFYSTAVSVYLQPPSPSSKDQGKMVSLFYGIIAMLNPLIYTLRNKEVKEGF
KRLVARVFLIKK

The disclosed NOV1b protein differs from the disclosed NOV1a protein at only one position. At positions 122, NOV1a has P, while NOV1b has W.

NOV1c

In the present invention, the target sequence identified previously, Accession Number dj408b20_B (NOV1a), was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly reactive was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species.

These primers were then employed in PCR amplification based on the following pool of human cDNAs: adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea, uterus. Usually, the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated NOV1c (Accession Number CG50369-01). This differs from the previously identified sequence of NOV1b (Accession Number dj408b20 B) in having 2 amino acid changes at the following positions: 270 (Gln→Arg) and 295 (Lys→Arg).

A disclosed NOV1c nucleic acid of 956 nucleotides is shown in Table 1E. An open reading frame was identified beginning with an ATG initiation codon at nucleotides 7-9 and ending with a TAA codon at nucleotides 946-948. Putative untranslated regions 5′ to the start codon and 3′ to the stop codon are underlined in Table 1E and the start and stop codons are in bold letters.

TABLE 1E
NOV1c Nucleic acid sequence (SEQ ID NO:5).

TTAATCATGAATTGGGTAAATGACAGCATCATACAGGAGTTTATTCTGCTGGGTTTCTCA
GATCGACCTTGGCTGGAGTTTCCAGTCCTTGTGGTCTTCTTGATTTCTTACACTGTGACC
ATCTTTGGCAATCTGACCATTATTCTAGTGTCACGCCTGGACACCAAACTTCATACCCCC
ATGTATTTTTTTCTTACCAATCTATCACTCCTGGATCTTTGTTACACCACATGTACAGTC
CCACAAATGCTAGTAAATTTATGCAGCATCAGGAAAGTAATCAGTTATCGTGGCTGTGTA
GCCCAGCTTTTCATATTTCTGGCCTTGGGGGCTACTGAATATCTTCTCCTGGCCGTCATG
TCCTTTGATAGGTTTGTAGCTATTTGTCGGCCTCTCCATTACTCAGTTATCATGCACCAG
AGACTCTGCCTCCAGTTGGCAGCTGCATCCTGGGTTACTGGTTTTAGTAACTCAGTGTGG
TTGTCTACCCTGACTCTCCAGCTGCCACTCTGTGACCCCTATGTGATAGATCACTTTCTC
TGTGAAGTCCCTGCACTGCTCAAGTTATCTTGTGTTGAGAGAACAGCAAATGAGGCTGAA
CTATTCCTTGTCAGTGAGCTCTTCCATCTAATACCCCTGACACTCATCCTTATATCATAT
GCTTTTATTGTCCGAGCAGTATTGAGGATACAGTCTGCTGAAGGTCGACAAAAAGCATTT
GGGACATGTGGTTCCCATCTAATTGTGGTGTCTCTTTTTTATAGTACAGCCGTCTCTGTG
TACCTGCAACCACCTTCGCCCAGCTCCAAGGACCGAGGAAAGATGGTTTCTCTCTTCTAT
GGAATCATTGCACCCATGCTGAATCCCCTTATATATACACTTAGGAACAGGGAGGTAAAG
GAAGGCTTTAAAAGGTTGGTTGCAAGAGTCTTCTTAATCAAGAAATAAGAAATATA

A disclosed NOV1c protein having 313 amino acid residues (SEQ ID NO:6) is presented using the one-letter code in Table 1F. An analysis using the PSORT program predicts that the disclosed NO1c protein localizes in the plasma membrane with a certainty=0.6000. It is also predicted that protein has a signal peptide whose most likely cleavage site is between residues 38 and 39, i.e., at the slash in the sequence TVT-1F.

TABLE 1F
Encoded NOV1c protein sequence (SEQ ID NO:6).

MNWVNDSIIQEFILLGFSDRPWLEFPLLVVFLISYTVTIFGNLTIILVSRLDTKLHTPMY
FFLTNLSLLDLCYTTCTVPQMLVNLCSIRKVISYRGCVAQLFIFLALGATEYLLLAVMSF
DRFVAICRPLHYSVINHQRLCLQLAAASVNTGFSNSVWLSTLTLQLPLCDPYVIDHFLCE
VPALLKLSCVETTANEAELFLVSELFHLIPLTLILISYAFIVRAVLRIQSAEGRQKAFGT
CGSHLIVVSLFYSTAVSVYLQPPSPSSKDRGKMVSLFYGIIAPMLNPLIYTLRNREVKEG
FKRLVARVFLIKK

The disclosed NOV1c sequence was derived by laboratory cloning of cDNA fragments covering the full length and/or part of the DNA sequence of the invention, and/or by in silico prediction of the full length and/or part of the DNA sequence of the invention from public human sequence databases. The laboratory cloning was performed using one or more of the methods summarized below:

SeqCallingTM Technology:

cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, steroids. The cDNA thus derived was then sequenced using CuraGen's proprietary SeqCalling technology. Sequence traces were evaluated manually and edited for corrections if appropriate. cDNA sequences from all samples were assembled with themselves and with public ESTs using bioinformatics programs to generate CuraGen's human SeqCalling database of SeqCalling assemblies. Each assembly contains one or more overlapping cDNA sequences derived from one or more human samples. Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations.

Exon Linking:

The cDNA coding for the sequence was cloned by polymerase chain reaction (PCR) using the following primers: GAATTGGGTAAATGACAGCATC (SEQ ID NO:32) and TTAGCAAAGCTTATCATTTGC (SEQ ID NO:33) on the following pool of human cDNAs: Pool 1—Adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea, uterus.

Primers were designed based on in silico predictions for the full length or part (one or more exons) of the DNA/protein sequence of the invention or by translated homology of the predicted exons to closely related human sequences or to sequences from other species. Usually multiple clones were sequenced to derive the sequence which was then assembled similar to the SeqCalling process. In addition, sequence traces were evaluated manually and edited for corrections if appropriate.

Physical Clone:

The PCR product derived by exon linking was cloned into the pCR2.1 vector from Invitrogen. The bacterial clone dj408b20_B.698008.B11 has an insert covering the entire open reading frame cloned into the pCR2.1 vector from Invitrogen.

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

NOV1c is expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, central nervous system and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources.

In the following positions, one or more consensus positions (Cons. Pos.) of the nucleotide sequence have been identified as SNPs. “Depth” rerepresents the number of clones covering the region of the SNP. The Putative Allele Frequency (Putative Allele Freq.) is the fraction of all the clones containing the SNP. The sign “>” means “is changed to”.

Cons.Pos.: 44 Depth: 43 Change: T>C

Putative Allele Freq.: 0.070

Cons.Pos.: 59 Depth: 43 Change: T>C

Putative Allele Freq.: 0.047

Cons.Pos.: 60 Depth: 43 Change: T>C

Putative Allele Freq.: 0.070

Cons.Pos.: 136 Depth: 41 Change: T>C

Putative Allele Freq.: 0.049

Cons.Pos.: 137 Depth: 41 Change: T>C

Putative Allele Freq.: 0.049

Cons.Pos.: 142 Depth: 41 Change: T>C

Putative Allele Freq.: 0.073

Cons.Pos.: 194 Depth: 41 Change: T>C

Putative Allele Freq.: 0.098

Cons.Pos.: 244 Depth: 41 Change: T>C

Putative Allele Freq.: 0.049

Cons.Pos.: 276 Depth: 41 Change: T>C

Putative Allele Freq.: 0.049

Cons.Pos.: 300 Depth: 41 Change: T>C

Putative Allele Freq.: 0.049

Cons.Pos.: 305 Depth: 41 Change: T>C

Putative Allele Freq.: 0.049

Cons.Pos.: 315 Depth: 41 Change: T>C

Putative Allele Freq.: 0.049

Cons.Pos.: 374 Depth: 49 Change: T>C

Putative Allele Freq.: 0.061

Cons.Pos.: 384 Depth: 51 Change: A>G

Putative Allele Freq.: 0.039

Cons.Pos.: 390 Depth: 55 Change: T>C

Putative Allele Freq.: 0.036

Cons.Pos.: 405 Depth: 56 Change: A>G

Putative Allele Freq.: 0.036

Cons.Pos.: 508 Depth: 60 Change: G>A

Putative Allele Freq.: 0.050

Cons.Pos.: 510 Depth: 60 Change: A>G

Putative Allele Freq.: 0.050

Cons.Pos.: 518 Depth: .59 Change: C>T

Putative Allele Freq.: 0.034

Cons.Pos.: 681 Depth: 39 Change: T>C

Putative Allele Freq.: 0.051

Cons.Pos.: 795 Depth: 40 Change: G>A

Putative Allele Freq.: 0.050

NOV1c may have important structural and/or physiological functions characteristic of the GPCR family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The NOV1 nucleic acids and proteins of the invention are useful in potential diagnostic and therapeutic applications implicated in various diseases and disorders described below and/or other pathologies. For example, the compositions of the present invention will have efficacy for treatment of patients suffering from: developmental diseases, MHCII and III diseases (immune diseases), taste and scent detectability disorders, Burkitt's lymphoma, corticoneurogenic disease, signal transduction pathway disorders, retinal diseases including those involving photoreception, cell growth rate disorders, cell shape disorders, feeding disorders, potential obesity due to over-eating, potential disorder due to starvation (lack of appetite), non-insulin-dependent diabetes mellitus (NIDDM1), bacterial, fungal, protozoal and viral infections (particularly infections caused by HIV-1 or HIV-2), pain, cancer (including but not limited to neoplasm; adenocarcinoma; lymphoma; prostate cancer; uterus cancer), anorexia, bulimia, asthma, allergies, Parkinson's disease, acute heart failure, hypotension, hypertension, urinary retention, osteoporosis, Crohn's disease, multiple sclerosis, Albright hereditary ostoeodystrophy, angina pectoris, myocardial infarction, ulcers, benign prostatic hypertrophy, psychotic and neurological disorders (including anxiety, schizophrenia, manic depression, delirium, dementia, and severe mental retardation), dentatorubro-pallidoluysian atrophy (DRPLA), hypophosphatemic rickets, autosomal dominant (2) acrocallosal syndrome and dyskinesias, such as Huntington's disease or Gilles de la Tourette syndrome and/or other pathologies and disorders of the like. The polypeptides can be used as immunogens to produce antibodies specific for the invention, and as vaccines. They can also be used to screen for potential agonist and antagonist compounds. For example, a cDNA encoding the OR-like protein may be useful in gene therapy, and the OR-like protein may be useful when administered to a subject in need thereof. By way of nonlimiting example, the compositions of the present invention will have efficacy for treatment of patients suffering from bacterial, fungal, protozoal and viral infections (particularly infections caused by HIV-1 or HIV-2), pain, cancer (including but not limited to neoplasm; adenocarcinoma; lymphoma; prostate cancer; uterus cancer), anorexia, asthma, allergies, bulimia, Parkinson's disease, acute heart failure, hypotension, hypertension, urinary retention, osteoporosis, Crohn's disease, multiple sclerosis; and Treatment of Albright hereditary ostoeodystrophy, angina pectoris, myocardial infarction, ulcers, benign prostatic hypertrophy, and psychotic and neurological disorders (including anxiety, schizophrenia, manic depression, delirium, dementia, severe mental retardation) and dyskinesias, such as Huntington's disease or Gilles de la Tourette syndrome and/or other pathologies and disorders. The novel nucleic acid encoding OR-like protein, and the OR-like protein of the invention, or fragments thereof, may further be useful in diagnostic applications, wherein the presence or amount of the nucleic acid or the protein are to be assessed.

The amino acids differences between the three NOV1 proteins are shown in Table 1G. The differences between the three proteins appear to be localized to a few distinct regions. Thus, these proteins may have similar functions, such as serving as olfactory or chemokine receptors (see below).

TABLE 1G
Differences for NOV1 Proteins.

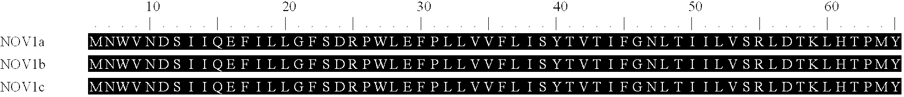

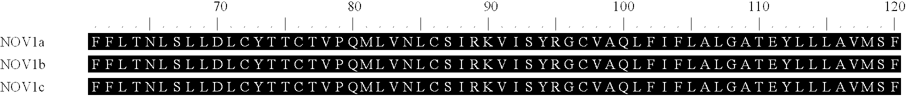

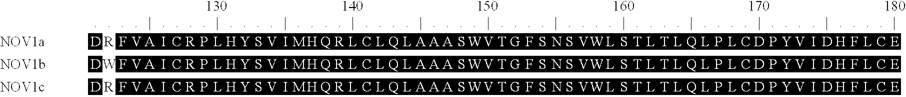

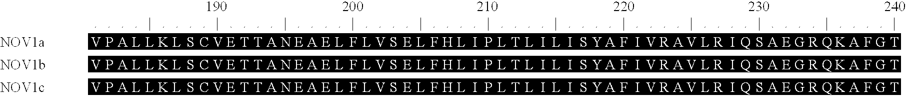

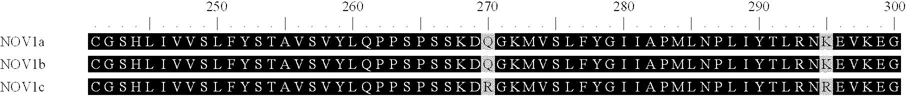

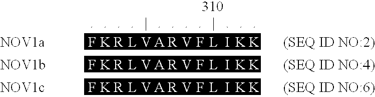

In all BLAST alignments described herein, the “E-value” or “Expect” value is a numeric indication of the probability that the aligned sequences could have achieved their similarity to the BLAST query sequence by chance alone, within the database that was searched. All BLAST analyses described herein were performed using the NOV1a amino acid sequence. As noted above, differences between the amino acid sequences of NOV1a, NOV1b, and NOV1c were limited to discrete amino acids (e.g., positions 122, 270, and 295).

A BLASTX search was performed against public protein databases. The full amino acid sequence of the protein of the invention was found to have 313 of 313 amino acid residues (100%) identical to, and 313 of 313 residues (100%) positive with, the 313 amino acid dJ408B20.2, a novel 7 transmembrane olfactory receptor protein from Homo sapien (emb|CAC14158.1 (AL133267), E=e-177). The disclosed NOV1a protein (SEQ ID NO:2) also has good identity with a number of olfactory receptor proteins. For example, the disclosed NOV1a has 254/310 (81%) amino acids identical with the 357 amino acid Homo sapien olfactory receptor (emb|CAC20513.1 (AJ302593) protein (Expect=e-143), and 253/310 (81%) amino acids identical, to the 310 amino acid Homo Sapien olfactory receptor (emb|CAB11427.1 (Z98744)) (Expect=e-142). The disclosed protein is also similar to the olfactory proteins disclosed in Table 1H.

TABLE 1H
BLAST results for NOV1.

| | | | | Posi- | |
|---|---|---|---|---|---|
| Gene Index/ | Protein/ | Length | Identity | tives | |
| Identifier | Organism | (aa) | (%) | (%) | Expect |
| Emb\|CAC14158.1 | DJ408B20.2 | 313 | 313/313 | 313/313 | e-177 |
| (AL133267) | (novel 7 | | (100%) | (100%) | |
| | trans- | | | | |
| | membrane | | | | |
| | receptor | | | | |
| | (olfactory | | | | |
| | family) | | | | |
| | (hS6M1-32) | | | | |
| | Homo sapien | | | | |
| Emb\|CAC20513.1 | Olfactory | 357 | 354/310 | 278/310 | e-143 |
| (AJ302593) | receptor | | (81%) | (88%) | |
| | Homo sapien | | | | |
| Emb\|CAB11427.1 | Olfactory | 310 | 253/310 | 278/310 | e-142 |
| (Z98744) | receptor | | (81%) | (89%) | |
| | Homo sapien | | | | |
| Emb\|CAC20504.1 | Olfactory | 357 | 253/310 | 278/310 | e-142 |
| (AJ302584) | receptor | | (81%) | (89%) | |
| | Homo sapien | | | | |
| Ref\|NP_0686321.1\| | OL1 receptor | 313 | 245/313 | 274/313 | e-138 |
| | Rattus | | (78%) | (87%) | |
| | norvegicus | | | | |

A ClustalW analysis comparing disclosed proteins of the invention with related OR protein sequences (as determined by BLASTX analysis) is given in Table 1I, with NOV1a shown on line 1.

TABLE 1I
ClustalW analysis of NOV1.

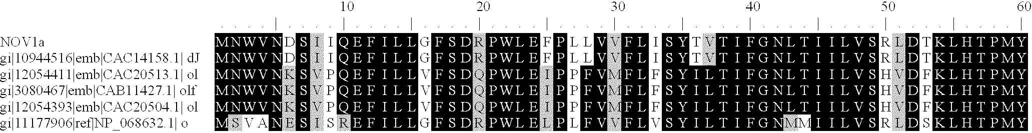

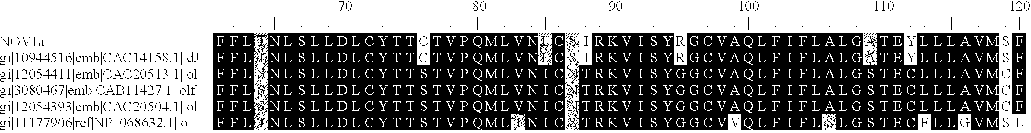

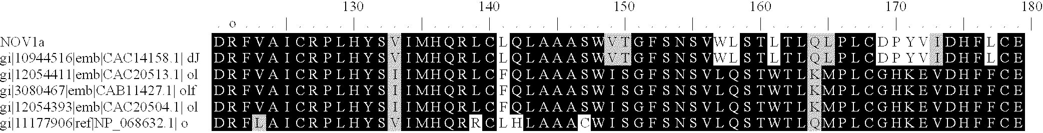

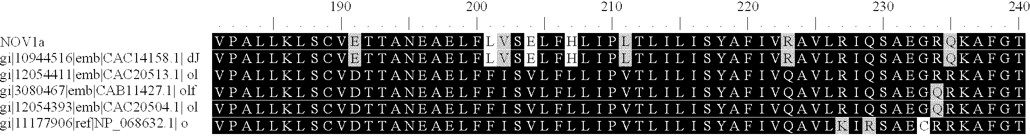

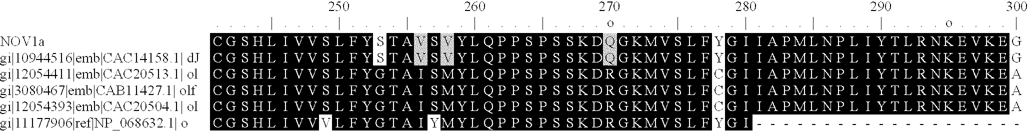

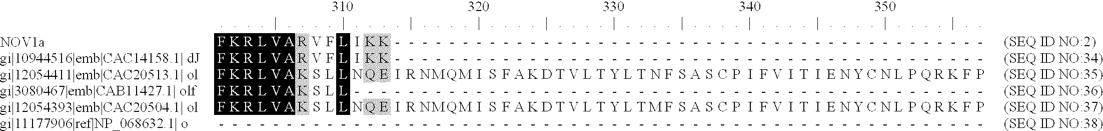

In the ClustalW alignment of the NOV1 protein, as well as all other ClustalW analyses described herein, the black outlined amino acid residues indicate regions of conserved sequence (i.e., regions that may be required to preserve structural or functional properties), whereas non-highlighted amino acid residues are less conserved and can potentially be mutated to a much broader extent without altering protein structure or function. Unless specifically addressed as NOV1a NOV1b, or NOV1c, any reference to NOV1 is assumed to encompass all variants. Residue differences between any NOVX variant sequences herein are written to show the residue in the “a” variant and the residue position with respect to the “a” variant. NOV residues in all following sequence alignments that differ between the individual NOV variants are marked with the (o) symbol above the variant residue in all alignments herein. For example, the protein shown in line 1 of Table 1I depicts the sequence for NOV1a, and the positions where NOV1b and NOV1c differs are marked with a (o) symbol (e.g., positions 122, 270, and 295).

The presence of identifiable domains in NOV1, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (hffp:www.ebi.ac.uk/interpro). DOMAIN results, e.g., for NOV1a, were collected from the Conserved Domain Database (CDD) with Reverse Position Specific BLAST analyses. This BLAST analysis software samples domains found in the Smart and Pfam collections. For all successive DOMAIN sequence alignments, fully conserved single residues are indicated by black shading and “strong” semi-conserved residues are indicated by grey shading. The “strong” group of conserved amino acid residues may be any one of the following groups of amino acids: STA, NEQK, NHQK, NDEQ, QHRK, MILV, MILF, HY, FYW.

The region from amino acid residue 41 through 243 (SEQ ID NO:2) most probably (E=3e⁻²⁵) contains a “seven transmembrane receptor (rhodopsin family) fragment” domain, aligned here with residues 1-187 of the 7tm_{—}1 entry (TM7, SEQ ID NO:39) of the Pfam database. This indicates that the NOV1 sequence has properties similar to those of other proteins known to contain this domain as well as to the 377 amino acid 7tm domain itself. NOV1 also has identity to another region of the TM7 protein. The region from amino acid residue 223 through 290 (of SEQ ID NO:2) aligns with amino acid residues 309-377 of TM7 (E=.003). Table 1J shows the results of the domain analysis of NOV1 and the TM7 protein.

TABLE 1J
Domain Analysis of NOV1.

Sbjct: 7 transmembrane receptor (rhodopsin family) fragment
Gnl|Pfam|pfam00001; Length = 377
Score = 108 bits (271), Expect = 3e−25

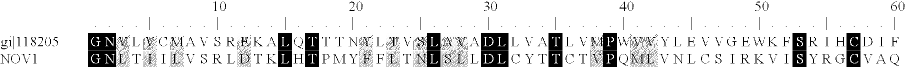

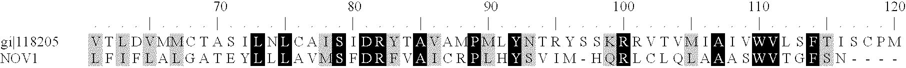

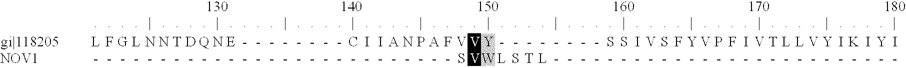

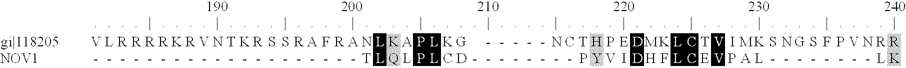

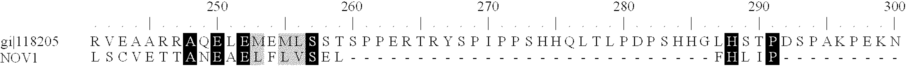

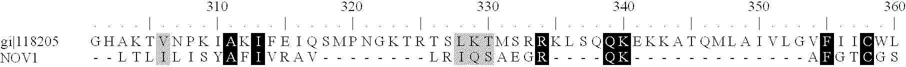

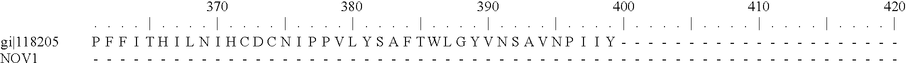

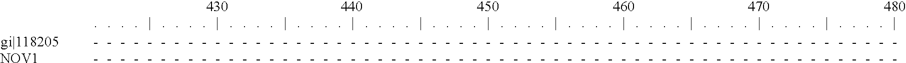

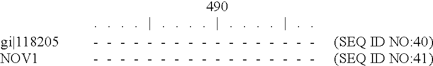

The representative member of the 7 transmembrane receptor family is the D2 dopamine receptor from Bos taurus (SWISSPROT: locus D2DR_BOVIN, accession P20288; gene index 118205). The D2 receptor is an integral membrane protein and belongs to Family 1 of G-protein coupled receptors. The activity of the D2 receptor is mediated by G proteins, which inhibit adenylyl cyclase. Chio et al., Nature 343:255-269 (1990). Residues 51-427 of this 444 amino acid protein are considered to be the representative TM7 domain, shown in Table 1K.

TABLE 1K
Amino Acid sequence for TM7 (SEQ ID NO:39).

GNVLVCMAVSREKALQTTTNYLIVSLAVADLLVATLVMPNVVYLEVVGEWKFSRIHCDIF
VTLDVMMCTASILNLCAISIDRYTAVAMPMLYNTRYSSKRRVTVMIAIVWVLSFTISCPM
LFGLNNTDQNECIIANPAFVVYSSIVSFYVPFIVTLLVYIKIYIVLRRRRKRVNTKRSSR
AFRANLKAPLKGNCTHPEDMKLCTVIMKSNGSFPVNRRRVEAARRAQELEMEMLSSTSPP
ERTRYSPIPPSHHQLTLPDPSHHGLHSTPDSPAKPEKNGHAKTVNPKIAKIFEIQSMPNG
KTRTSLKTMSRRKLSQQKEKKATQMLAIVLGVFIICWLPFFITHILNIHCDCNIPPVLYS
AFTWLGYVNSAVNPIIY

The 7 transmembrane receptor family includes a number of different proteins, including, for example, serotonin receptors, dopamine receptors, histamine receptors, andrenergic receptors, cannabinoid receptors, angiotensin II receptors, chemokine receptors, opioid receptors, G-protein coupled receptor (GPCR) proteins, olfactory receptors (OR), and the like. Some proteins and the Protein Data Base Ids/gene indexes include, for example: rhodopsin (129209); 5-hydroxytryptamine receptors; (112821, 8488960, 112805, 231454, 1168221, 398971, 112806); G protein-coupled receptors (119130, 543823, 1730143, 132206, 137159, 6136153, 416926, 1169881, 136882, 134079); gustatory receptors (544463, 462208); c-x-c chemokine receptors (416718, 128999, 416802, 548703, 1352335); opsins (129193, 129197, 129203); and olfactory receptor-like proteins (129091, 1171893, 400672, 548417).

Expression information for NOV1 RNA was derived using tissue sources including, but not limited to, proprietary database sources, public EST sources, literature sources, and/or RACE sources.

The nucleic acids and proteins of NOV1 are useful in potential therapeutic applications implicated in various NOV- or olfactory receptor (OR)-related pathologies and/or disorders. For example, a cDNA encoding the G-protein coupled receptor-like protein may be useful in gene therapy, and the G-protein coupled receptor-like protein may be useful when administered to a subject in need thereof. The novel nucleic acid encoding NOV1 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. The NOVX nucleic acids and proteins are useful in potential diagnostic and therapeutic applications implicated in various diseases and disorders described below and/or other pathologies. For example, the compositions of the present invention will have efficacy for treatment of patients suffering from: cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, fertility, hemophilia, hypercoagulation, idiopathic thrombocytopenic purpura, immunodeficiencies, graft versus host disease, bronchial asthma, and other diseases, disorders and conditions of the like. By way of nonlimiting example, the compositions of the present invention will have efficacy for treatment of patients suffering from neoplasm, adenocarcinoma, lymphoma, prostate cancer, uterus cancer, immune response, AIDS, asthma, Crohn's disease, multiple sclerosis, and Albright Hereditary Ostoeodystrophy. Additional NOV-related diseases and disorders are mentioned throughout the Specification.

Further, the protein similarity information, expression pattern, and map location for NOV1 suggests that NOV1 may have important structural and/or physiological functions characteristic of the NOV family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immuno-specifically to the novel NOV1 substances for use in therapeutic or diagnostic methods. These antibodies may be generated according to methods known in the art, using prediction from a hydrophobicity charts, as described in the “Anti-NOVX Antibodies” section below.

NOV2

NOV2 includes a family of four novel G-protein coupled receptor (“GPCR”) proteins disclosed below. The disclosed proteins have been named NOV2a, NOV2b, NOV2c, and NOV2d and are each related to olfactory receptors.

NOV2a

A disclosed NOV2a nucleic acid of 960 (designated CuraGen Acc. No. 6-L-9-D) nucleotides is shown in Table 2A. The disclosed NOV2a open reading frame (“ORF”) begins at the ATG initiation codon at nucleotides 14-16, shown in bold in Table 2A. The encoded polypeptide is referred to herein as NOV2a polypeptide. The disclosed NOV2a ORF terminates at a TAG codon at nucleotides 955-957. As shown in Table 2A, putative untranslated regions 5′ to the start codon and 3′ to the stop codon are underlined, and start and stop codons are in bold letters.

TABLE 2A
NOV2a Nucleotide Sequence (SEQ ID NO:7).

CCAACAAGTATCATGATGGCATCTGAAAGAAATCAAAGCAGCACACCCACTTTTATTCTCTTGGGTTTTT
CAGAATACCCAGAAATCCAGGTTCCACTCTTTCTGGTTTTCTTGTTCGTCTACACAGTCACTGTAGTGGG
GAACTTGGGCATGATAATAATCATCAGACTCAATTCAAAACTCCATACAATCATGTACTTTTTCCTTAGT
CACTTGTCCTTGACAGACTTCTGTTTTTCCACTGTAGTTACACCTAAACTGTTGGAGAACTTGGTTGTGG
AATACAGAACCATCTCTTTCTCTGGTTGCATCATGCAATTTTGTTTTGCTTGCATTTTTGGAGTGACAGA
AACTTTCATGTTAGCAGCGATGGCTTATGACCGTTTTGTGGCAGTTTGTAAACCCTTGCTGTATACCACT
ATTATGTCTCAGAAGCTCTGTGCTCTTCTGGTGGCTGGGTCCTATACATGGGGGATAGTGTGCTCCCTGA
TACTCACATATTTTCTTCTTGACTTATCGTTTTGTGAATCTACCTTCATAAATAATTTTATCTGTGACCA
CTCTGTAATTGTTTCTGCCTCCTACTCAGACCCCTATATCAGCCAGAGGCTATGCTTTATTATTGCCATA
TTCAATGAGGTGAGCAGCCTAATTATCATTCTGACATCATATATGCTTATTTTCACTACCATTATGAAGA
TGCGATCTGCAAGTGGGCGCCAGAAAACTTTCTCCACCTGTGCCTCCCACCTGACAGCCATCACTATCTT
CCATGGAACTATCCTTTTCCTTTACTGTGTTCCTAATCCTAAAACTTCTAGCCTCATAGTTACAGTGGCT
TCTGTGTTTTACACAGTGGCGATTCCAATGCTGAACCCATTGATCTACAGCCTTAGGAACAAAGATATCA
ATAACATGTTTGAAAAATTAGTTGTCACCAAATTGATTTACCACTGAATA

An open reading frame (ORF) for NOV2a was identified from nucleotides 14 to 955. The disclosed NOV2a polypeptide (SEQ ID NO:8) encoded by SEQ ID NO:7 is 314 amino acid residues and is presented using the one-letter code in Table 2B. The NOV2a protein was analyzed for signal peptide prediction and cellular localization. SignalPep results predict that NOV2a is cleaved between position 43 and 44 of SEQ ID NO:8, i.e., at the slash in the amino acid sequence VVG-NL. Psort and Hydropathy profiles also predict that NOV2a contains a signal peptide and is likely to be localized at the plasma membrane (certainty of 0.6000) or possibly to Golgi body with a lower certainty.

TABLE 2B
Encoded NOV2a protein sequence (SEQ ID NO:8).

MMASERNQSSTPTFILLGFSEYPEIQVPLFLVFLFVYTVTVVGNLGMIIIIRLNSKLHTIMYFFLSHLSL
TDFCFSTVVTPKLLENLVVEYRTISFSGCIMQFCFACIFGVTETFMLAAMAYDRFVAVCKPLLYTTIMSQ
KLCALLVAGSYTWGIVCSLILTYFLLDLSFCESTFINNFICDHSVIVSASYSDPYISQRLCFIIAIFNEV
SSLIIILTSYMLIFTTIMKMRSASGRQKTFSTCASHLTAITIFHGTILFLYCVPNPKTSSLIVTVASVFY
TVAIPMLNPLIYSLRNKDINNMFEKLVVTKLIYH

A novel nucleic acid was identified on chromosome 11 by BlastN using CuraGen Corporation's sequence file for GPCR probe or homolog, run against the Genomic Daily Files made available by GenBank. The nucleic acid was further predicted by the program GenScan™, including selection of exons. These were further modified by means of similarities using BLAST searches. The sequences were then manually corrected for apparent inconsistencies, thereby obtaining the sequences encoding the full-length protein.

NOV2b

A target sequence identified previously as NOV2a (Accession Number 6_L_{—}19_D) was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such suitable sequences were then employed as the forward and reverse primers in a PCR amplification based on a wide range of cDNA libraries. The resulting amplicon was gel purified, cloned and sequenced to high redundancy to provide the sequence reported below, which is designated NOV2b (Accession Number 6_L_{—}19_D_da1). The resulting nucleotide sequence differs from that of NOV2a at nucleotides 344 and 900. The encoded protein differs from that of NOV2a at residue 113.

A disclosed NOV2b nucleic acid of 954 nucleotides is shown in Table 2C. The disclosed NOV2b open reading frame (“ORF”) begins at the ATG initiation codon at nucleotides 7-9, shown in bold in Table 2C. The encoded polypeptide is referred to herein as NOV2b polypeptide. The disclosed NOV2b ORF terminates at a TGA codon at nucleotides 949-951. As shown in Table 2C, putative untranslated regions 5′ to the start codon and 3′ to the stop codon are underlined, and the start and stop codons are in bold letters.

TABLE 2C
NOV2b Nucleotide Sequence (SEQ ID NO:9)

AGTATCATGATGGCATCTGAAAGAAATCAAAGCAGCACACCCACTTTTATTCTCTTGGGTTTTTCAGAAT
ACCCAGAAATCCAGGTTCCACTCTTTCTGGTTTTCTTGTTCGTCTACACAGTCACTGTAGTGGGGAACTT
GGGCATGATAATAATCATCAGACTCAATTCAAAACTCCATACAATCATGTACTTTTTCCTTAGTCACTTG
TCCTTGACAGACTTCTGTTTTTCCACTGTAGTTACACCTAAACTGTTGGAGAACTTGGTTGTGGAATACA
GAACCATCTCTTTCTCTGGTTGCATCATGCAATTTTGTTTTGCTTGCATTTTTGGAGTGACAGGAACTTT
CATGTTAGCAGCGATGGCTTATGACCGTTTTGTGGCAGTTTGTAAACCCTTGCTGTATACCACTATTATG
TCTCAGAAGCTCTGTGCTCTTCTGGTGGCTGGGTCCTATACATGGGGGATAGTGTGCTCCCTGATACTCA
CATATTTTCTTCTTGACTTATCGTTTTGTGAATCTACCTTCATAAATAATTTTATCTGTGACCACTCTGT
AATTGTTTCTGCCTCCTACTCAGACCCCTATATCAGCCAGAGGCTATGCTTTATTATTGCCATATTCAAT
GAGGTGAGCAGCCTAATTATCATTCTGACATCATATATGCTTATTTTCACTACCATTATGAAGATGCGAT
CTGCAAGTGGGCGCCAGAAAACTTTCTCCACCTGTGCCTCCCACCTGACAGCCATCACTATCTTCCATGG
AACTATCCTTTTCCTTTACTGTGTTCCTAATCCTAAAACTTCTAGCCTCATAGTTACAGTGGCTTCTGTG
TTTTACACAGTGGCGATTCCAATGCTGAACCCATTGATCTACAGCCTTAGGAACAAAGACATCAATAACA
TGTTTGAAAAATTAGTTGTCACCAAATTGATTTACCACTGAATA

An open reading frame (ORF) for NOV2b was identified from nucleotides 7 to 949. The disclosed NOV2b polypeptide (SEQ ID NO:10) encoded by SEQ ID NO:9 is 314 amino acid residues and is presented using the one-letter code in Table 2D. The NOV2b protein was analyzed for signal peptide prediction and cellular localization. SignalPep results predict that NOV2b is cleaved between position 43 and 44 of SEQ ID NO:10, i.e., at the slash in the amino acid sequence VVG-NL. Psort and Hydropathy profiles also predict that NOV2b contains a signal peptide and is likely to be localized at the plasma membrane (certainty of 0.6000).

TABLE 2D
Encoded NOV2b protein sequence (SEQ ID NO:10).

MMASERNQSSTPTFILLGFSEYPEIQVPLFLVFLFVYTVTVVGNLGMIIIIRLNSKLHTIMYFFLSHLSL
TDFCFSTVVTPKLLENLVVEYRTISFSGCIMQFCFACIFGVTGTFMLAAMAYDRFVAVCKPLLYTTIMSQ
KLCALLVAGSYTWGIVCSLILTYFLLDLSFCESTFINNFICDHSVIVSASYSDPYISQRLCFIIAIFNEV
SSLIIILTSYMLIFTTIMKMRSASGRQKTFSTCASHLTAITIFHGTILFLYCVPNPKTSSLIVTVASVFY
TVAIPMLNPLIYSLRNKDINNMFEKLVVTKLIYH

NOV2c

A target sequence identified previously as NOV2a (Accession Number 6_L_{—}19_D) was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species.

These primers were then employed in PCR amplification based on the following pool of human cDNAs: adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequences reported below, which are designated NOV2c and NOV2d. NOV2c and NOV2d differ from the previously identified sequence (NOV2a) at bp: 344, 386, 900 (NOV2d; 6_L_{—}19_D_da1)) and 344, 386 (NOV2c; 6_L_{—}19_D_da2).

A disclosed NOV2c nucleic acid of 954 nucleotides is shown in Table 2E. The disclosed NOV2c open reading frame (“ORF”) begins at the ATG initiation codon at nucleotides 7-9, shown in bold in Table 2E. The encoded polypeptide is referred to herein as NOV2c polypeptide. The disclosed NOV2c ORF terminates at a TGA codon at nucleotides 949-951. As shown in Table 2E, putative untranslated regions 5′ to the start codon and 3′ to the stop codon are underlined, and the start and stop codons are in bold letters.

TABLE 2E
NOV2c Nucleotide Sequence (SEQ ID NO:11).

AGTATCATGATGGCATCTGAAAGAAATCAAAGCAGCACACCCACTTTTATTCTCTTGGGTTTTTCAGAAT
ACCCAGAAATCCAGGTTCCACTCTTTCTGGTTTTCTTGTTCGTCTACACAGTCACTGTAGTGGGGAACTT
GGGCATGATAATAATCATCAGACTCAATTCAAAACTCCATACAATCATGTACTTTTTCCTTAGTCACTTG
TCCTTGACAGACTTCTGTTTTTCCACTGTAGTTACACCTAAACTGTTGGAGAACTTGGTTGTGGAATACA
GAACCATCTCTTTCTCTGGTTGCATCATGCAATTTTGTTTTGCTTGCATTTTTGGAGTGACAGGAACTTT
CATGTTAGCAGCGATGGCTTATGACCGTTTTGTGGTAGTTTGTAAACCCTTGCTGTATACCACTATTATG
TCTCAGAAGCTCTGTGCTCTTCTGGTGGCTGGGTCCTATACATGGGGGATAGTGTGCTCCCTGATACTCA
CATATTTTCTTCTTGACTTATCGTTTTGTGAATCTACCTTCATAAATAATTTTATCTGTGACCACTCTGT
AATTGTTTCTGCCTCCTACTCAGACCCCTATATCAGCCAGAGGCTATGCTTTATTATTGCCATATTCAAT
GAGGTGAGCAGCCTAATTATCATTCTGACATCATATATGCTTATTTTCACTACCATTATGAAGATGCGAT
CTGCAAGTGGGCGCCAGAAAACTTTCTCCACCTGTGCCTCCCACCTGACAGCCATCACTATCTTCCATGG
AACTATCCTTTTCCTTTACTGTGTTCCTAATCCTAAAACTTCTAGCCTCATAGTTACAGTGGCTTCTGTG
TTTTACACAGTGGCGATTCCAATGCTGAACCCATTGATCTACAGCCTTAGGAACAAAGACATCAATAACA
TGTTTGAAAAATTAGTTGTCACCAAATTGATTTACCACTGAATA

An open reading frame (ORF) for NOV2c was identified from nucleotides 7 to 949. The disclosed NOV2c polypeptide (SEQ ID NO: 12) encoded by SEQ ID NO:11 is 314 amino acid residues and is presented using the one-letter code in Table 2F. The NOV2c protein was analyzed for signal peptide prediction and cellular localization. SignalPep results predict that NOV2c is cleaved between position 43 and 44 of SEQ ID NO:12, i.e., at the slash in the amino acid sequence VVG-NL. Psort and Hydropathy profiles also predict that NOV2b contains a signal peptide and is likely to be localized at the plasma membrane (certainty of 0.6000).

TABLE 2F
Encoded NOV2c protein sequence (SEQ ID NO:12).

MMASERNQSSTPTFILLGFSEYPEIQVPLFLVFLFVYTVTVVGNLGMIIIIRLNSKLHTIMYFFLSHLSL
TDFCFSTVVTPKLLENLVVEYRTISFSGCIMQFCFACIFGVTGTFMLAAMAYDRFVVVCKPLLYTTIMSQ
KLCALLVAGSYTWGIVCSLILTYFLLDLSFCESTFINNFICDHSVIVSASYSDPYISQRLCFIIAIFNEV
SSLIIILTSYMLIFTTIMKMRSASGRQKTFSTCASHLTAITIFHGTILFLYCVPNPKTSSLIVTVASVFY
TVAIPMLNPLIYSLRNKDINNMFEKLVVTTKLIYH

Possible SNPs for the disclosed NOV2c include:

345: A→G(2)

126549454(i), phred 49

126728467(i), phred 40

387: C→T(2)

128360465(i), phred 45

128360641(i), phred 33

489: T→C(2)

126549471(i), phred 29

126597292(i), phred 28

901: C→T(2)

128791738(i), phred 41

128791725(i), phred 28

345: A→G(2)

126549454(i), phred 49

126728467(i), phred 40

387: C→T(2)

128360465(i), phred 45

128360641(i), phred 33

489: T→C(2)

126549471(i), phred 29

126597292(i), phred 28

901: C→T(2)

128791738(i), phred 41

128791725(i), phred 28

NOV2d

A disclosed NOV2d nucleic acid of 954 nucleotides is shown in Table 2G. The disclosed NOV2d open reading frame (“ORF”) begins at the ATG initiation codon at nucleotides 7-9, shown in bold in Table 2G. The encoded polypeptide is referred to herein as NOV2d polypeptide. The disclosed NOV2d ORF terminates at a TGA codon at nucleotides 949-951. As shown in Table 2G, putative untranslated regions 5′ to the start codon and 3′ to the stop codon are underlined, and the start and stop codons are in bold letters. The nucleic acid sequence of NOV2d differs from NOV2b at position 386, which is C in NOV2b and T in NOV2d.

TABLE 2G
NOV2d Nucleotide Sequence (SEQ ID NO:13).

AGTATCATGATGGCATCTGAAAGAAATCAAAGCAGCACACCCACTTTTATTCTCTTGGGTTTTTCAGAATACCCAGAAAT
CCAGGTTCCACTCTTTCTGGTTTTCTTGTTCGTCTACACAGTCACTGTAGTGGGGAACTTGGGCATGATAATAATCATCA
GACTCAATTCAAAACTCCATACAATCATGTACTTTTTCCTTAGTCACTTGTCCTTGACAGACTTCTGTTTTTCCACTGTA
GTTACACCTAAACTGTTGGAGAACTTGGTTGTGGAATACAGAACCATCTCTTTCTCTGGTTGCATCATGCAATTTTGTTT
TGCTTGCATTTTTGGAGTGACAGGAACTTTCATGTTAGCAGCGATGGCTTATGACCGTTTTGTGGTAGTTTGTAAACCCT
TGCTGTATACCACTATTATGTCTCAGAAGCTCTGTGCTCTTCTGGTGGCTGGGTCCTATACATGGGGGATAGTGTGCTCC
CTGATACTCACATATTTTCTTCTTGACTTATCGTTTTGTGAATCTACCTTCATAAATAATTTTATCTGTGACCACTCTGT
AATTGTTTCTGCCTCCTACTCAGACCCCTATATGAGCCAGAGGCTATGCTTTATTATTGCCATATTCAATGAGGTGAGCA
GCCTAATTATCATTCTGACATCATATATGCTTATTTTCACTACCATTATGAAGATGCGATCTGCAAGTGGGCGCCAGAAA
ACTTTCTCCACCTGTGCCTCCCACCTGACAGCCATCACTATCTTCCATGGAACTATCCTTTTCCTTTACTGTGTTCCTAA
TCCTAAAACTTCTAGCCTCATAGTTACAGTGGCTTCTGTGTTTTACACAGTGGCGATTCCAATGCTGAACCCATTGATCT
ACAGCCTTAGGAACAAGACATCAATAACATGTTTGAAAATTAGTTGTCACCAAATTGATTTACCACTGAATA

An open reading frame (ORF) for NOV2d was identified from nucleotides 7 to 949. The disclosed NOV2d polypeptide (SEQ ID NO:14) encoded by SEQ ID NO:13 is 311 amino acid residues and is presented using the one-letter code in Table 2H. The NOV2d protein was analyzed for signal peptide prediction and cellular localization. SignalPep results predict that NOV2d is cleaved between position 43 and 44 of SEQ ID NO:14, i.e., at the slash in the amino acid sequence VVG-NL. Psort and Hydropathy profiles also predict that NOV2b contains a signal peptide and is likely to be localized at the plasma membrane (certainty of 0.6000).

TABLE 2H
Encoded NOV2d protein sequence (SEQ ID NO:14).

MMASERNQSSTPTFILLGFSEYPEIQVPLFLVFLFVYTVTVVGNLGMIIIIRLNSKLHTIMYFFLSHLSLTDFCFST
VVTPKLLENLVVEYRTISFSGCIMQFCFACIFGVTGTFMLAAMAYDRFVVVCKPLLYTTIMSQKLCALLVAGSYTWG
IVCSLILTYFLLDLSFCESTFINNFICDHSVIVSASYSDPYISQRLCFIIAIFNEVSSLIIILTSYMLIFTTIMKMR
SASGRQKTFSTCASHLTAITIFHGTILFLYCVPNPKTSSLIVTVASVFYTVAIPMLNPLIYSLRNKDINNMFEKLVV
TKLIYH

Possible SNPs for the disclosed NOV2d include:

62: A→G(2)

125509355(i), phred 37

125585624(i), phred 51

221: T→C(3)

125509328(i), phred 45

125531417(i), phred 37

125585593(i), phred 39

NOV2 nucleic acids and proteins of the invention are useful in potential therapeutic applications implicated in used in the treatment of infections such as bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The polypeptides can be used as immunogens to produce antibodies specific for the invention, and as vaccines. They can also be used to screen for potential agonist and antagonist compounds. For example, a cDNA encoding the GPCR-like protein may be useful in gene therapy, and the GPCR-like protein may be useful when administered to a subject in need thereof. By way of nonlimiting example, the compositions of the present invention will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The novel nucleic acid encoding GPCR-like protein, and the GPCR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The amino acids differences between three of the NOV2 proteins are shown in Table 2I. The differences between the three proteins appear to be localized to a few distinct regions. Thus, these proteins may have similar functions, such as serving as olfactory or chemokine receptors (see below).

[image]

In all BLAST alignments described herein, the “E-value” or “Expect” value is a numeric indication of the probability that the aligned sequences could have achieved their similarity to the BLAST query sequence by chance alone, within the database that was searched. All BLAST analyses described herein were performed using the NOV2a amino acid sequence. As noted above, differences between the nucleic acid sequences of NOV2a, NOV2b, NOV2c, and NOV2d were limited to discrete nucleotides (e.g., positions 344, 386, and 900).

A BLASTX search was performed against public protein databases. The amino acid sequence of the protein of the invention was found to have 159 of 214 amino acid residues (74%) identical to, and 181 of 214 residues (84%) positive with, a 216 amino acid Homo sapien olfactory receptor protein (gi|381598|gbAAC70015.1| (AF065860), E=2e-69). The disclosed protein is also similar to the olfactory proteins disclosed in Table 2J.

TABLE 2J
BLAST results for NOV2.

| Gene Index/ | Protein/ | Length | Identity | Positives | |
|---|---|---|---|---|---|
| Identifier | Organism | (aa) | (%) | (%) | Expect |
| Gi \| 3831598 \| gb \| | Olfactory | 216 | 159/214 | 181/214 | 2e−69 |
| AAC70015.1 | receptor | | (74%) | (84%) | |
| (AF065860) | Homo sapien | | | | |
| Gi \| 9297024 \| sp \| | HUMAN OLFACTORY | 216 | 158/214 | 180/214 | 7e−69 |
| Q9UP62 \| O5D4_HUMAN | RECEPTOR 5D4 | | (73%) | (83%) | |
| | (OLFACTORY | | | | |
| | RECEPTOR 11-8C) | | | | |
| | (OR11-8C) | | | | |
| Gi \| 7211249 \| gb \| | Olfactory | 216 | 153/214 | 175/214 | 3e−66 |
| AAF0259.1 | receptor | | (71%) | (81%) | |
| (AF127840) | Hylobates lar | | | | |
| Gi \| 1246534 \| emb \| | Olfactory | 312 | 143/304 | 200/304 | 3e−61 |
| CAA64370.1 \| | receptor 4 | | (47%) | (65%) | |
| (X94744) | Gallus gallus | | | | |
| Gi \| 1246530 \| emb \| | Olfactory | 332 | 138/312 | 198/312 | 5e−60 |
| CAA64368.1 | receptor 2 | | (44%) | (63%) | |
| (X94742) | Gallus gallus | | | | |

A ClustalW analysis comparing disclosed proteins of the invention with related OR protein sequences (as determined by BLASTX analysis) is given in Table 2K, with NOV2a shown on line 1.

[image]

[image]

In the ClustalW alignment of the NOV2 protein, as well as all other ClustalW analyses described herein, the black outlined amino acid residues indicate regions of conserved sequence (i.e., regions that may be required to preserve structural or functional properties), whereas non-highlighted amino acid residues are less conserved and can potentially be mutated to a much broader extent without altering protein structure or function. Unless specifically addressed as NOV2a, NOV2b, NOV2c, or NOV2d, any reference to NOV2 is assumed to encompass all variants. Residue differences between any NOVX variant sequences herein are written to show the residue in the “a” variant and the residue position with respect to the “a” variant. NOV residues in all following sequence alignments that differ between the individual NOV variants are marked with the (o) symbol above the variant residue in all alignments herein. For example, the protein shown in line 1 of Table 2K depicts the sequence for NOV2a, and the positions where NOV2b, NOV2c, and NOV2d differ are marked with a (o) symbol (e.g., positions 130 and 147).

The presence of identifiable domains in NOV2, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results, e.g., for NOV2a, were collected from the Conserved Domain Database (CDD) with Reverse Position Specific BLAST analyses. This BLAST analysis software samples domains found in the Smart and Pfam collections. For all successive DOMAIN sequence alignments, fully conserved single residues are indicated by black shading and “strong” semi-conserved residues are indicated by grey shading. The “strong” group of conserved amino acid residues may be any one of the following groups of amino acids: STA, NEQK, NHQK, NDEQ, QHRK, MILV, MILF, HY, FYW.

The region from amino acid residue 43 through 231 (SEQ ID NO:8) most probably (E=4e⁻¹⁹) contains a “seven transmembrane receptor (rhodopsin family) fragment” domain, aligned here with residues 1-170 of the 7tm_{—}1 entry (TM7, SEQ ID NO:39) of the Pfam database. This indicates that the NOV2 sequence has properties similar to those of other proteins known to contain this domain as well as to the 377 amino acid 7tm domain itself. Table 2L shows the results of the NOV2 and the TM7 protein.

[image]

The representative member of the 7 transmembrane receptor family is the D2 dopamine receptor from Bos taurus (SWISSPROT: locus D2DR_BOVIN, accession P20288; gene index 118205). The D2 receptor is an integral membrane protein and belongs to Family 1 of G-protein coupled receptors. The activity of the D2 receptor is mediated by G proteins, which inhibit adenylyl cyclase. Chio et al., Nature 43:255-269 (1990). Residues 51-427 of this 444 amino acid protein are considered to be representative TM7 domain, shown in Table 2M.

TABLE 2M
Amino Acid sequence for TM7 (SEQ ID NO:39).

GNVLVCMAVSREKALQTTTNYLIVSLAVADLLVATLVMPWVVYLEVVGEWKFSRIHCDIF
VTLDVMMCTASILNLCAISIDRYTAVAMPMLYNTRYSSKRRVTVMIAIVWVLSFTISCPM
LFGLNNTDQNECIIANPAFVVYSSIVSFYVPFIVTLLVYIKIYIVLRRRRKRVNTKRSSR
AFRANLKAPLKGNCTHPEDMKLCTVIMKSNGSFPVNRRRVEAARRAQELEMEMLSSTSPP
ERTRYSPIPPSHHQLTLPDPSHHGLHSTPDSPAKPEKNGHAKTVNPKIAKIFEIQSMPNG
KTRTSLKTMSRRKLSQQKEKKATQMLAIVLGVFIICWLPFFITHILNIHCDCNIPPVLYS
AFTWLGYVNSAVNPIIY

The 7 transmembrane receptor family includes a number of different proteins, including, for example, serotonin receptors, dopamine receptors, histamine receptors, andrenergic receptors, cannabinoid receptors, angiotensin II receptors, chemokine receptors, opioid receptors, G-protein coupled receptor (GPCR) proteins, olfactory receptors (OR), and the like. Some proteins and the Protein Data Base Ids/gene indexes include, for example: rhodopsin (129209); 5-hydroxytryptamine receptors; (112821, 8488960, 112805, 231454, 1168221, 398971, 112806); G protein-coupled receptors (119130, 543823, 1730143, 132206, 137159, 6136153, 416926, 1169881, 136882, 134079); gustatory receptors (544463, 462208); c-x-c chemokine receptors (416718, 128999, 416802, 548703, 1352335); opsins (129193, 129197, 129203); and olfactory receptor-like proteins (129091, 1171893, 400672, 548417).

The NOV2 homology to the olfactory receptors suggests that an endogenous small molecule ligand regulates this gene and hence drugs structurally similar to the endogenous ligand could serve as agonists and antagonists to regulate the biological effects of NOV2.

The nucleic acids and proteins of NOV2 are useful in potential therapeutic applications implicated in various NOV- or olfactory receptor (OR)-related pathologies and/or disorders. For example, a cDNA encoding the G-protein coupled receptor-like protein may be useful in gene therapy, and the G-protein coupled receptor-like protein may be useful when administered to a subject in need thereof. The novel nucleic acid encoding NOV2 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. The NOVX nucleic acids and proteins are useful in potential diagnostic and therapeutic applications implicated in various diseases and disorders described below and/or other pathologies. For example, the compositions of the present invention will have efficacy for treatment of patients suffering from: cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, fertility, hemophilia, hypercoagulation, idiopathic thrombocytopenic purpura, immunodeficiencies, graft versus host disease, bronchial asthma, and other diseases, disorders and conditions of the like. By way of nonlimiting example, the compositions of the present invention will have efficacy for treatment of patients suffering from neoplasm, adenocarcinoma, lymphoma, prostate cancer, uterus cancer, immune response, AIDS, asthma, Crohn's disease, multiple sclerosis, and Albright Hereditary Ostoeodystrophy. Additional NOV-related diseases and disorders are mentioned throughout the Specification.

Further, the protein similarity information, expression pattern, and map location for NOV2 suggests that NOV2 may have important structural and/or physiological functions characteristic of the NOV family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immuno-specifically to the novel NOV2 substances for use in therapeutic or diagnostic methods. These antibodies may be generated according to methods known in the art, using prediction from hydrophobicity charts, as described in the “Anti-NOVX Antibodies” section below.

NOV3

An additional NOV-like protein of the invention, referred to herein as NOV3, is an Olfactory Receptor (“OR”)-like protein. A novel nucleic acid was identified on chromosome 11 by TblastN using CuraGen Corporation's sequence file for GPCR probe or homolog, run against the Genomic Daily Files made available by GenBank. The nucleic acid was further predicted by the program GenScan™, including selection of exons. These were further modified by means of similarities using BLAST searches. The sequences were then manually corrected for apparent inconsistencies, thereby obtaining the sequences encoding the full-length protein.

The novel nucleic acid of 957 nucleotides (6-L-19-E, SEQ ID NO:15) encoding a novel olfactory receptor-like protein is shown in Table 3A. An open reading frame (ORF) was identified beginning with an ATG initiation codon at nucleotides 13-15 and ending with a TAA codon at nucleotides 955-957. Putative untranslated regions upstream from the initiation codon and downstream from the termination codon are underlined in Table 3A, and the start and stop codons are in bold letters.

TABLE 3A
NOV3 Nucleotide Sequence (SEQ ID NO:15).

CTAACAACAACCATGTCATTAGCTGAAGGAAATCAGAGTTCTGGAGCCGTATTTACCCTCTTGGGCTTCT
CAGAATATGCAGACCTCCAGGTTCCTCTGTTCCTGGTCTTCCTGACCATCTACACAATCACTGTATTGGG
AAACCTGGGCATGATCATGATCATCAGGATCAACCCCAAACTCCACACCCGCATGTACTTTTTCCTCAGC
CACTTGTCCTTTGTTGATTTCTGTTATTCCACCACAGTTACACCCAAACTGCTGGAGAACTTGGTTGTGG
AAGACAGAACCATCTCCTTCACAGGATGCATCATGCAATTCTTCCTGGCGTGTATATGTGCAGTGGCAGA
AACATTCATGCTGGCAGTGATGGCCTATGATAGATACGTGGCAGTGTGTAACCCTTTGCTCTACACAGTT
GTCAGGTCCCAGAAACTCTGTGCATCATTAGTGGCAGGGCCCTACACATGGGGTATAATCTCTTCTCTGA
CACTCACCTATTTCCTCTTGTCATTATCCTTCTGTGGGTCTAACATCATCAATAATTTTGTCTGTGAGCA
CTCTGTCATCATCTCTGTCTCCTGCTCTGACCCCTACATCAGCCAAATGCTTTGTTTTGTCATTGCAATA
TTCAATGAGGTGAGCAGCTTGGGAGTCATCCTCACTACCTATATTTTCATCTTTATTGCTGTCATAAAAA
TGCCTTCTGCTGTTGGGCACCAAAAAGCTTTCTCTACCTGTGCTTCCCACCTGACTGCCATCACTATTTT
CCACGGGACTGTCCTGTTCCTTTATTGTGTACCCAACTCCAAAAACTCATGGCTCATAGTCAAAGTAGGT
TCTGTGTTTTATACAGTCATCATCCCCACGTTGAACCCTTTAACCTACAGCCTCAGGAACAAAGACGTGA
AAGAGAGTGTTCGAAAGTTAATGAATCACTCAATACAATTTTGTTAAAGA

A disclosed NOV3 polypeptide (SEQ ID NO:16) encoded by SEQ ID NO:15 is 314 amino acid residues and is presented using the one-letter code in Table 3B. The disclosed NOV3 protein was analyzed for signal peptide prediction and cellular localization. SignalP results predict that NOV3 is cleaved between position 43 and 44 of SEQ ID NO: 16, i.e., at the slash in the amino acid sequence VLG-NL. Psort and Hydropathy profiles also predict that NOV3 contains a signal peptide and is likely to be localized to the endoplasmic reticulum (membrane) or the plasma membrane with comparable, high certainties.

TABLE 3B
Encoded NOV3 protein sequence (SEQ ID NO:16).

MSLAEGNQSSGAVFTLLGFSEYADLQVPLFLVFLTIYTITVLGNLGMIMIIRINPKLHTRMYFFLSHLSF
VDFCYSTTVTPKLLENLVVEDRTISFTGCIMQFFLACICAVAETFMLAVMAYDRYVAVCNPLLYTVVRSQ
KLCASLVAGPYTWGIISSLTLTYFLLSLSFCGSNIINNFVCEHSVIISVSCSDPYISQMLCFVIAIFNEV
SSLGVILTTYIFIFIAVIKMPSAVGHQKAFSTCASHLTAITIFHGTVLFLYCVPNSKNSWLIVKVGSVFY
TVIIPTLNPLTYSLRNKDVKESVRKLMNHSIQFC

The NOV3 nucleic acids and proteins of the invention are useful in potential therapeutic applications implicated in used in the treatment of infections such as bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The polypeptides can be used as immunogens to produce antibodies specific for the invention, and as vaccines. They can also be used to screen for potential agonist and antagonist compounds. For example, a cDNA encoding the GPCR-like protein may be useful in gene therapy, and the GPCR-like protein may be useful when administered to a subject in need thereof. By way of nonlimiting example, the compositions of the present invention will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The novel nucleic acid encoding GPCR-like protein, and the GPCR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

In all BLAST alignments described herein, the “E-value” or “Expect” value is a numeric indication of the probability that the aligned sequences could have achieved their similarity to the BLAST query sequence by chance alone, within the database that was searched. All BLAST analyses described herein were performed using the NOV3 amino acid sequence.

A BLASTX search was performed against public protein databases. The amino acid sequence of the protein of the invention was found to have 153 of 216 amino acid residues (70%) identical to, and 175 of 216 residues (80%) positive with, the 216 amino acid Homo sapien human olfactory receptor 5D4 (Olfactory Receptor 11-8C (OR11-8C) (gi|9297024|sp|Q9UP62|O5D4, E=7e-73). The disclosed protein is also similar to the olfactory proteins disclosed in Table 3C.

TABLE 3C
BLAST results for NOV3.

| | | | | Posi- | |
|---|---|---|---|---|---|
| Gene Index/ | Protein/ | Length | Identity | tives | |
| Identifier | Organism | (aa) | (%) | (%) | Expect |
| Gi\|9297024\|sp\| | HUMAN | 216 | 153/216 | 175/216 | 7e-73 |
| Q9UP62\|O5D4 | OLFACTORY | | (70%) | (80%) | |
| | RECEPTOR | | | | |
| | 5D4 | | | | |
| | (OLFACTORY | | | | |
| | RECEPTOR | | | | |
| | 11-8C) | | | | |
| | (OR11-8C) | | | | |
| Gi\|3831598\|gb\| | Olfactory | 216 | 153/216 | 175/216 | 8e-73 |
| AAC70015.1 | receptor | | (70%) | (80%) | |
| (AF065860) | Homo sapien | | | | |
| Gi\|7211249\|gb\| | Olfactory | 216 | 155/216 | 174/216 | 5e-72 |
| AAF40259.1\| | receptor | | (71%) | (79%) | |
| (AF127840) | Hylobates lar | | | | |
| Gi\|1246534\|emb\| | Olfactory | 312 | 148/305 | 208/305 | 2e-70 |
| CAA64370.1 | receptor 4 | | (48%) | (67%) | |
| | Gallus gallus | | | | |
| Gi\|1246530\|emb\| | Olfactory | 332 | 139/305 | 203/305 | 6e-69 |
| CAA64368.1\| | receptor 2 | | (45%) | (65%) | |
| (X94742) | Gallus gallus | | | | |

A ClustalW analysis comparing disclosed proteins of the invention with related OR protein sequences (as determined by BLASTX analysis) is given in Table 3D, with NOV3 shown on line 1.

[image]

[image]

In the ClustalW alignment of the NOV3 protein, as well as all other ClustalW analyses described herein, the black outlined amino acid residues indicate regions of conserved sequence (i.e., regions that may be required to preserve structural or functional properties), whereas non-highlighted amino acid residues are less conserved and can potentially be mutated to a much broader extent without altering protein structure or function.

The presence of identifiable domains in NOV3, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results, e.g., for NOV3, were collected from the Conserved Domain Database (CDD) with Reverse Position Specific BLAST analyses. This BLAST analysis software samples domains found in the Smart and Pfam collections. For all successive DOMAIN sequence alignments, fully conserved single residues are indicated by black shading and “strong” semi-conserved residues are indicated by grey shading. The “strong” group of conserved amino acid residues may be any one of the following groups of amino acids: STA, NEQK, NHQK, NDEQ, QHRK, MILV, MILF, HY, FYW.

The region from amino acid residue 43 through 238 (SEQ ID NO:15) most probably (E=4e⁻¹⁷) contains a “seven transmembrane receptor (rhodopsin family) fragment” domain, aligned here with residues 1-177 of the 7tm_{—}1 entry (TM7, SEQ ID NO:39) of the Pfam database. This indicates that the NOV3 sequence has properties similar to those of other proteins known to contain this domain as well as to the 377 amino acid 7tm domain itself. Table 3E shows the results of the NOV3 and the TM7 protein.

[image]

The representative member of the 7 transmembrane receptor family is the D2 dopamine receptor from Bos taurus (SWISSPROT: locus D2DR_BOVIN, accession P20288; gene index 118205). The D2 receptor is an integral membrane protein and belongs to Family 1 of G-protein coupled receptors. The activity of the D2 receptor is mediated by G proteins, which inhibit adenylyl cyclase. Chio et al., Nature 343:255-269 (1990). Residues 51-427 of this 444 amino acid protein are considered to be the representative TM7 domain, shown in Table 3F.

TABLE 3F
Amino Acid sequence for TM7 (SEQ ID NO:39).

GNVLVCMAVSREKALQTTTNYLIVSLAVADLLVATLVMPWVVYLEVVGEWKFSRIHCDIF
VTLDVMMCTASILNLCAISIDRYTAVAMPMLYNTRYSSKRRVTVMIAIVWVLSFTISCPM
LFGLNNTDQNECIIANPAFVVYSSIVSFYVPFIVTLLVYIKIYIVLRRRRKRVNTKRSSR
AFRANLKAPLKGNCTHPEDMKLCTVIMKSNGSFPVNRRRVEAARRAQELEMEMLSSTSPP
ERTRYSPIPPSHHQLTLPDPSHHGLHSTPDSPAKPEKNGHAKTVNPKIAKIFEIQSMPNG
KTRTSLKTMSRRKLSQQKEKKATQMLAIVLGVFIICWLPFFITHILNIHCDCNIPPVLYS
AFTWLGYVNSAVNPIIY

The 7 transmembrane receptor family includes a number of different proteins, including, for example, serotonin receptors, dopamine receptors, histamine receptors, andrenergic receptors, cannabinoid receptors, angiotensin II receptors, chemokine receptors, opioid receptors, G-protein coupled receptor (GPCR) proteins, olfactory receptors (OR), and the like. Some proteins and the Protein Data Base Ids/gene indexes include, for example: rhodopsin (129209); 5-hydroxytryptamine receptors; (112821, 8488960, 112805, 231454, 1168221, 398971, 112806); G protein-coupled receptors (119130, 543823, 1730143, 132206, 137159, 6136153, 416926, 1169881, 136882, 134079); gustatory receptors (544463, 462208); c-x-c chemokine receptors (416718, 128999, 416802, 548703, 1352335); opsins (129193, 129197, 129203); and olfactory receptor-like proteins (129091, 1171893, 400672, 548417).

The nucleic acids and proteins of NOV3 are useful in potential therapeutic applications implicated in various NOV- or olfactory receptor (OR)-related pathologies and/or disorders. For example, a cDNA encoding the G-protein coupled receptor-like protein may be useful in gene therapy, and the G-protein coupled receptor-like protein may be useful when administered to a subject in need thereof. The novel nucleic acid encoding NOV3 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. The NOVX nucleic acids and proteins are useful in potential diagnostic and therapeutic applications implicated in various diseases and disorders described below and/or other pathologies. For example, the compositions of the present invention will have efficacy for treatment of patients suffering from: cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, fertility, hemophilia, hypercoagulation, idiopathic thrombocytopenic purpura, immunodeficiencies, graft versus host disease, bronchial asthma, and other diseases, disorders and conditions of the like. By way of nonlimiting example, the compositions of the present invention will have efficacy for treatment of patients suffering from neoplasm, adenocarcinoma, lymphoma, prostate cancer, uterus cancer, immune response, AIDS, asthma, Crohn's disease, multiple sclerosis, and Albright Hereditary Ostoeodystrophy. Additional NOV-related diseases and disorders are mentioned throughout the Specification.

Further, the protein similarity information, expression pattern, and map location for NOV3 suggests that NOV3 may have important structural and/or physiological functions characteristic of the NOV family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immuno-specifically to the novel NOV3 substances for use in therapeutic or diagnostic methods. These antibodies may be generated according to methods known in the art, using prediction from hydrophobicity charts, as described in the “Anti-NOVX Antibodies” section below.

NOV4

NOV4 includes a family of two nucleic acids disclosed below. The disclosed nucleic acids encode a NOV-like protein.

NOV4a

A novel nucleic acid was identified on chromosome 11 by TblastN using CuraGen Corporation's sequence file for GPCR probe or homolog, run against the Genomic Daily Files made available by GenBank. The nucleic acid was further predicted by the program GenScan™, including selection of exons. These were further modified by means of similarities using BLAST searches. The sequences were then manually corrected for apparent inconsistencies, thereby obtaining the sequences encoding the full-length protein.

A disclosed NOV4a nucleic acid of 968 nucleotides (95-h-6-A, SEQ ID NO:17) encoding a novel olfactory receptor-like protein is shown in Table 4A. An open reading frame (ORF) was identified beginning with an ATG initiation codon at nucleotides 8-10 and ending with a TAA codon at nucleotides 956-958. Putative untranslated regions upstream from the initiation codon and downstream from the termination codon are underlined in Table 4A, and the start and stop codons are in bold letters.

TABLE 4A
NOV4a Nucleotide Sequence (SEQ ID NO:17).

GGGAAACATGCAAAACCAAAGCTTTGTAACTGAGTTTGTCCTCCTGGGACTTTCACAGAATCCAAATGTT
CAGGAAATAGTATTTGTTGTATTTTTGTTTGTCTACATTGCAACTGTTGGGGGCAACATGCTAATTGTAG
TAACCATTCTCACCAGCCCTGCTCTTCTGGTGTCTCCTATGTACTTCTTCTTGGGCTTCCTGTCCTTCCT
GGATGCGTGCTTCTCATCTGTCATCACCCCAAAGATGATTGTAGACTCCCTCTATGTGACAAAAACCATC
TCTTTTGAAGGCTGCATGATGCAGCTCTTTGCTGAACACTTCTTTGCTGGGGTGGAGGTGATTGTCCTCA
CAGCCATGGCCTATGATCGTTATGTGGCCATTTGCAAGCCCTTGCATTACTCTTCTATCATGAACAGGAG
GCTCTGTGGCATTCTGATGGGGGTAGCCTGGACAGGGGGCCTCTTGCATTCCATGATACAAATTCTTTTT
ACTTTCCAGCTTCCCTTTTGTGGCCCCAATGTCATCAATCACTTTATGTGTGACTTGTACCCGTTACTGG
AGCTTGCCTGCACTGATACTCACATCTTTGGCCTCATGGTGGTCATCAACAGTGGGTTTATCTGCATCAT
AAACTTCTCCTTGTTGCTTGTCTCCTATGCTGTCATCTTGCTCTCTCTGAGAACACACAGTTCTGAAGGG
CGCTGGAAAGCTCTCTCCACCTGTGGATCTCACATTGCTGTTGTGATTTTGTTCTTTGTCCCATGCATAT
TTGTATATACACGACCTCCATCTGCTTTTTCCCTTGACAAAATGGCGGCAATATTTTATATCATCTTAAA
TCCCTTGCTCAATCCTTTGATTTACACTTTCAGGAATAAGGAAGTAAAACAGGCCATGAGGAGAATATGG
AACAGACTGATGGTGGTTTCTGATGAGAGAAAGAAAATATTAAACTTTAAAAAATCCAAA

A disclosed NOV4a polypeptide (SEQ ID NO:18) encoded by SEQ ID NO:17 is 316 amino acid residues and is presented using the one-letter code in Table 4B. The disclosed NOV4a protein was analyzed for signal peptide prediction and cellular localization. SignalP results predict that NOV4a is cleaved between position 39 and 40 of SEQ ID NO:18, i.e., at the slash in the amino acid sequence VGG-NM. Psort and Hydropathy profiles also predict that NOV4a contains a signal peptide and is likely to be localized in the plasma membrane (certainty=0.6000).

TABLE 4B
Encoded NOV4 protein sequence (SEQ ID NO:18).

MQNQSFVTEFVLLGLSQNPNVQEIVFVVFLFVYIATVGGNMLIVVTILSSPALLVSPMYFFLGFLSFLDA
CFSSVITPKMIVDSLYVTKTISFEGCMMQLFAEHFFAGVEVIVLTAMAYDRYVAICKPLHYSSIMNRRLC
GILMGVAWTGGLLHSMIQILFTFQLPFCGPNVINHFMCDLYPLLELACTDTHIFGLMVVINSGFICIINF
SLLLVSYAVILLSLRTHSSEGRWKALSTCGSHIAVVILFFVPCIFVYTRPPSAFSLDKMAAIFYIILNPL
LNPLIYTFRNKEVKQAMRRIWNRLMVVSDEKENIKL

NOV4b

A target sequence identified previously as NOV4a (Accession Number 95-h-6-A) was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such suitable sequences were then employed as the forward and reverse primers in a PCR amplification based on a wide range of cDNA libraries. The resulting amplicon was gel purified, cloned and sequenced to high redundancy to provide the sequence reported below, which is designated NOV4b (Accession Number 95-h-6-A1). There is a single nucleotide difference between the two clones at base 274 that results in no change in amino acid sequence.

A disclosed NOV4b nucleic acid of 968 nucleotides (SEQ ID NO:19) encoding a novel olfactory receptor-like protein is shown in Table 4C. An open reading frame (ORF) was identified beginning with an ATG initiation codon at nucleotides 8-10 and ending with a TAA codon at nucleotides 956-958. Putative untranslated regions upstream from the initiation codon and downstream from the termination codon are underlined in Table 4C, and the start and stop codons are in bold letters.

TABLE 4C
NOV4b Nucleotide Sequence (SEQ ID NO:19).

GGGAAACATGCAAAACCAAAGCTTTGTAACTGAGTTTGTCCTCCTGGGACTTTCACAGAATCCAAATGTTCAGGAAATAG
TATTTGTTGTATTTTTGTTTGTCTACATTGCAACTGTTGGGGGCAACATGCTAATTGTAGTAACCATTCTCAGCAGCCCT
GCTCTTCTGGTGTCTCCTATGTACTTCTTCTTGGGCTTCCTGTCCTTCCTGGATGCGTGCTTCTCATCTGTCATCACCCC
AAAGATGATTGTAGACTCCCTCTATGTGACAAAGACCATCTCTTTTGAAGGCTGCATGATGCAGCTCTTTGCTGAACACT
TCTTTGCTGGGGTGGAGGTGATTGTCCTCACAGCCATGGCCTATGATCGTTATGTGGCCATTTGCAAGCCCTTGCATTAC
TCTTCTATCATGAACAGGAGGCTCTGTGGCATTCTGATGGGGGTAGCCTGGACAGGGGGCCTCTTGCATTCCATGATACA
AATTCTTTTTACTTTCCAGCTTCCCTTTTGTGGCCCCAATGTCATCAATCACTTTATGTGTGACTTGTACCCGTTACTGG
AGCTTGCCTGCACTGATACTCACATCTTTGGCCTCATGGTGGTCATCAACAGTGGGTTTATCTGCATCATAAACTTCTCC
TTGTTGCTTGTCTCCTATGCTGTCATCTTGCTCTCTCTGAGAACACACAGTTCTGAAGGGCGCTGGAAAGCTCTCTCCAC
CTGTGGATCTCACATTGCTGTTGTGATTTTGTTCTTTGTCCCATGCATATTTGTATATACACGACCTCCATCTGCTTTTT
CCCTTGACAAAATGGCGGCAATATTTTATATCATCTTAAATCCCTTGCTCAATCCTTTGATTTACACTTTCAGGAATAAG
GAAGTAAAACAGGCCATGAGGAGAATATGGAACAGACTGATGGTGGTTTCTGATGAGAAAGAAAATATTAAACTTTAAAA
AATCCAAA

The disclosed NOV4b polypeptide is identical to the disclosed NOV4a polypeptide (SEQ ID NO:18). This polypeptide has 316 amino acid residues and is presented using the one-letter code in Table 4B. The disclosed NOV4 protein was analyzed for signal peptide prediction and cellular localization. SignalP results predict that NOV4 is cleaved between position 39 and 40 of SEQ ID NO:18, i.e., at the slash in the amino acid sequence VGG-NM. Psort and Hydropathy profiles also predict that NOV4 contains a signal peptide and is likely to be localized in the plasma membrane (certainty=0.6000).

The NOV4 nucleic acids and proteins of the invention are useful in potential therapeutic applications implicated in used in the treatment of infections such as bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The polypeptides can be used as immunogens to produce antibodies specific for the invention, and as vaccines. They can also be used to screen for potential agonist and antagonist compounds. For example, a cDNA encoding the GPCR-like protein may be useful in gene therapy, and the GPCR-like protein may be useful when administered to a subject in need thereof. By way of nonlimiting example, the compositions of the present invention will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The novel nucleic acid encoding GPCR-like protein, and the GPCR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of this invention for use in therapeutic or diagnostic methods.

The amino acid differences between the two NOV4 proteins are shown in Table 4D. Thus, these proteins may have similar functions, such as serving as olfactory or chemokine receptors (see below).

[image]

The nucleotide differences between the two NOV4 nucleic acids are shown in Table 4E. The differences between the nucleic acids appear to be localized to a few distinct regions.

[image]

[image]

[image]

In all BLAST alignments described herein, the “E-value” or “Expect” value is a numeric indication of the probability that the aligned sequences could have achieved their similarity to the BLAST query sequence by chance alone, within the database that was searched. All BLAST analyses described herein were performed using the NOV4a amino acid sequence. As noted above, there are no differences between the amino acid sequences of NOV4a and NOV4b.

A BLASTX search was performed against public protein databases. The amino acid sequence of the NOV4 protein of the invention was found to have 187 of 301 amino acid residues (62%) identical to, 228 of 301 residues (70%) positive, and 1 gap out of 301 residues, with, the 308 amino acid odorant receptor 16, an olfactory receptor protein from Mus musculus (gi|11496249|ref|NP_{—}067343.1, E=1e-93). The disclosed NOV4 protein (SEQ ID NO:18) also has good identity with a number of olfactory receptor proteins. The disclosed protein is also similar to the olfactory proteins disclosed in Table 4F.

TABLE 4F
BLAST results for NOV4.

| | | | | Posi- | | |
|---|---|---|---|---|---|---|
| Gene Index/ | Protein/ | Length | Identity | tives | Ex- | |
| Identifier | Organism | (aa) | (%) | (%) | pect | Gap |
| Gi\|11496249\|ref\| | Odorant | 308 | 187/301 | 228/301 | 1e-93 | 1/301 |
| NP_067343.1\| | receptor | | (62%) | (75%) | | |
| | 16 | | | | | |
| | Mus | | | | | |
| | musculus | | | | | |
| Gi\|11464995\|ref\| | Gene for | 302 | 182/303 | 227/303 | 3e-91 | 1/303 |
| NP_065261.1\|; | odorant | | (60%) | (74%) | | |
| Gi\|6178010\|dbj\| | receptor | | | | | |
| BAA86127.1 | A16 | | | | | |
| (AB030896) | Mus | | | | | |
| | musculus; | | | | | |
| | Odorant | | | | | |
| | receptor | | | | | |
| | A16 | | | | | |
| | Mus | | | | | |
| | musculus | | | | | |
| Gi\|423702\|pir∥S2 | Olfactory | 307 | 177/305 | 226/305 | 2e-85 | 1/305 |
| 9710 | receptor | | (58%) | (74%) | | |
| | OR18 Rat | | | | | |
| Gi}11463993\|ref\| | Gene for | 308 | 157/301 | 210/301 | 1e-72 | 1/301 |
| NP_065260.1\| | odorant | | (52%) | (69%) | | |
| Gi\|6178006\|dbj\| | receptor | | | | | |
| BAA86125.1 | MOR83 | | | | | |
| (AB030894) | Mus | | | | | |
| | musculus; | | | | | |
| | Odorant | | | | | |
| | receptor | | | | | |
| | MOR83 | | | | | |
| | Mus | | | | | |
| | musculus | | | | | |
| Gi\|3983372\|gb\| | Olfactory | 220 | 146/211 | 169/211 | 6e-71 | — |
| AAD13314.1 | receptor | | (69%) | (79%) | | |
| (AF102522) | C3 | | | | | |
| | Mus | | | | | |
| | musculus | | | | | |

A ClustalW analysis comparing disclosed proteins of the invention with related OR protein sequences (as determined by BLASTX analysis) is given in Table 4G, with NOV4 shown on line 1.

[image]

In the ClustalW alignment of the NOV4 protein, as well as all other ClustalW analyses described herein, the black outlined amino acid residues indicate regions of conserved sequence (i.e., regions that may be required to preserve structural or functional properties), whereas non-highlighted amino acid residues are less conserved and can potentially be mutated to a much broader extent without altering protein structure or function. Unless specifically addressed as NOV4a or NOV4b, any reference to NOV4 is assumed to encompass all variants.

The presence of identifiable domains in NOV4, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results, e.g., for NOV4a, were collected from the Conserved Domain Database (CDD) with Reverse Position Specific BLAST analyses. This BLAST analysis software samples domains found in the Smart and Pfam collections. For all successive DOMAIN sequence alignments, fully conserved single residues are indicated by black shading and “strong” semi-conserved residues are indicated by grey shading. The “strong” group of conserved amino acid residues may be any one of the following groups of amino acids: STA, NEQK, NHQK, NDEQ, QHRK, MILV, MILF, HY, FYW.

The region from amino acid residue 39 through 231 (SEQ ID NO: 18) most probably (E=2e⁻²¹) contains a “seven transmembrane receptor (rhodopsin family) fragment” domain, aligned here with residues 1-173 of the 7tm_{—}1 entry (TM7, SEQ ID NO:39) of the Pfam database. This indicates that the NOV4 sequence has properties similar to those of other proteins known to contain this domain as well as to the 377 amino acid 7tm domain itself. Table 4H shows the results of the domain analysis of the NOV4 and the TM7 protein.

[image]

[image]

The representative member of the 7 transmembrane receptor family is the D2 dopamine receptor from Bos taurus (SWISSPROT: locus D2DR_BOVIN, accession P20288; gene index 118205). The D2 receptor is an integral membrane protein and belongs to Family 1 of G-protein coupled receptors. The activity of the D2 receptor is mediated by G proteins, which inhibit adenylyl cyclase. Chio et al., Nature 343:255-269 (1990). Residues 51-427 of this 444 amino acid protein are considered to be the representative TM7 domain, shown in Table 41.

TABLE 4I

| | |
|---|---|
| Amino Acid sequence for TM7 | (SEQ ID NO:39). |

GNVLVCMAVSREKALQTTTNYLIVSLAVADLLVATLVMPWVVYLEVVGEWKFSRIHCDIF
VTLDVMMCTASILNLCAISIDRYTAVAMPMLYNTRYSSKRRVTVMIAIVWVLSFTISCPM
LFGLNNTDQNECIIANPAFVVYSSIVSFYVPFIVTLLVYIKIYIVLRRRRKRVNTKRSSR
AFRANLKAPLKGNCTHPEDMKLCTVIMKSNGSFPVNRRRVEAARRAQELEMEMLSSTSPP
ERTRYSPIPPSHHQLTLPDPSHHGLHSTPDSPAKPEKNGHAKTVNPKIAKIFEIQSMPNG
KTRTSLKTMSRRKLSQQKEKKATQMLAIVLGVFIICWLPFFITHILNIHCDCNIPPVLYS
AFTWLGYVNSAVNPIIY

The 7 transmembrane receptor family includes a number of different proteins, including, for example, serotonin receptors, dopamine receptors, histamine receptors, andrenergic receptors, cannabinoid receptors, angiotensin II receptors, chemokine receptors, opioid receptors, G-protein coupled receptor (GPCR) proteins, olfactory receptors (OR), and the like. Some proteins and the Protein Data Base Ids/gene indexes include, for example: rhodopsin (129209); 5-hydroxytryptamine receptors; (112821, 8488960, 112805, 231454, 1168221, 398971, 112806); G protein-coupled receptors (119130, 543823, 1730143, 132206, 137159, 6136153, 416926, 1169881, 136882, 134079); gustatory receptors (544463, 462208); c-x-c chemokine receptors (416718, 128999, 416802, 548703, 1352335); opsins (129193, 129197, 129203); and olfactory receptor-like proteins (129091, 1171893, 400672, 548417).

The nucleic acids and proteins of NOV4 are useful in potential therapeutic applications implicated in various NOV- or olfactory receptor (OR)-related pathologies and/or disorders. For example, a cDNA encoding the G-protein coupled receptor-like protein may be useful in gene therapy, and the G-protein coupled receptor-like protein may be useful when administered to a subject in need thereof. The novel nucleic acid encoding NOV4 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. The NOVX nucleic acids and proteins are useful in potential diagnostic and therapeutic applications implicated in various diseases and disorders described below and/or other pathologies. For example, the compositions of the present invention will have efficacy for treatment of patients suffering from: cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, fertility, hemophilia, hypercoagulation, idiopathic thrombocytopenic purpura, immunodeficiencies, graft versus host disease, bronchial asthma, and other diseases, disorders and conditions of the like. By way of nonlimiting example, the compositions of the present invention will have efficacy for treatment of patients suffering from neoplasm, adenocarcinoma, lymphoma, prostate cancer, uterus cancer, immune response, AIDS, asthma, Crohn's disease, multiple sclerosis, and Albright Hereditary Ostoeodystrophy. Additional NOV-related diseases and disorders are mentioned throughout the Specification.

Further, the protein similarity information, expression pattern, and map location for NOV4 suggests that NOV4 may have important structural and/or physiological functions characteristic of the NOV family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immuno-specifically to the novel NOV4 substances for use in therapeutic or diagnostic methods. These antibodies may be generated according to methods known in the art, using prediction from hydrophobicity charts, as described in the “Anti-NOVX Antibodies” section below.

NOV5

A novel nucleic acid was identified on chromosome 11 by TblastN using CuraGen Corporation's sequence file for GPCR probe or homolog, run against the Genomic Daily Files made available by GenBank. The nucleic acid was further predicted by the program GenScan™, including selection of exons. These were further modified by means of similarities using BLAST searches. The sequences were then manually corrected for apparent inconsistencies, thereby obtaining the sequences encoding the full-length protein.

A disclosed nucleic acid of 946 nucleotides (95-h-6-B, SEQ ID NO:20) encoding a novel NOV5 olfactory receptor-like protein is shown in Table 5A. An open reading frame (ORF) was identified beginning with an ATG initiation codon at nucleotides 7-9 and ending with a TAA codon at nucleotides 937-939. Putative untranslated regions upstream from the initiation codon and downstream from the termination codon are underlined in Table 5A, and the start and stop codons are in bold letters.

TABLE 5A
NOV5 Nucleotide Sequence (SEQ ID NO:20)

AAAACCATGCAACTGAATAATAATGTGACTGAGTTCATTCTGCTTGGATTGACACAGGATCCTTTTTGGA
AGAAAATAGTGTTTGTTATTTTTTTGCGTCTCTACTTGGGAACACTGTTGGGTAATTTGCTAATCATTAT
TAGTGTCAAGGCCAGCCAGGCACTTAAGAACCCAATGTTCTTCTTCCTTTTCTACTTATCTTTATCTGAT
ACTTGCCTCTCTACTTCCATAGCCCCTAGAATGATTGTGGATGCCCTTTTGAAGAAGACAACTATCTCCT
TCAGCGAGTGCATGATCCAAGTCTTTTCATCCCATGTCTTTGGCTGCCTGGAGATCTTCATCCTCATCCT
CACGGCTGTTGACCGCTATGTGGACATCTGTAAGCCCCTGCACTACATGACCATCATAAGCCAGTGGGTC
TGTGGTGTTTTGATGGCTGTGGCCTGGGTGGGATCCTGTGTGCATTCTTTAGTTCAGATTTTTCTTGCCC
TGAGTTTGCCATTCTGTGGCCCCAATGTGATCAATCACTGTTTCTGTGACTTGCAGCCCTTGTTGAAACA
AGCCTGTTCAGAAACCTATGTGGTTAACCTACTCCTGGTTTCCAATAGTGGGGCCATTTGTGCAGTGAGT
TATGTCATGCTAATATTCTCCTATGTCATCTTCTTGCATTCTCTGAGAAACCACAGTGCTGAAGTGATAA
AGAAAGCACTTTCCACATGTGTCTCCCACATCATTGTGGTCATCTTGTTCTTTGGACCTTGCATATTTAT
GTACACATGCCCTGCAACCGTATTCCCCATGGATAAGATGATAGCTGTATTTTATACAGTTGGAACATCT
TTTCTCAACCCTGTGATTTACACGCTGAAGAATACAGAAGTGAAAAGTGCCATGAGGAAGCTTTGGAGCA
ACAAATTGATCACAGATGACAAAAGATAAATGAAGG

A disclosed NOV5 polypeptide (SEQ ID NO:21) encoded by SEQ ID NO:20 is 308 amino acid residues and is presented using the one-letter code in Table 5B. The disclosed NOV5 protein was analyzed for signal peptide prediction and cellular localization. SignalP results predict that NOV5 is cleaved between position 49 and 50 of SEQ ID NO:21, i.e., at the slash in the amino acid sequence VKA-SQ. Psort and Hydropathy profiles also predict that NOV5 contains a signal peptide and is localized in the plasma membrane (certainty=0.6000).

TABLE 5B
Encoded NOV5 protein sequence (SEQ ID NO:21).

MQLNNNVTEFILLGLTQDPFWKKIVFVIFLRLYLGTLLGNLLIIISVKASQALKNPMFFFLFYLSLSDTC
LSTSIAPRMIVDALLKKTTISFSECMIQVFSSHVFGCLEIFILILTAVDRYVDICKPLHYMTIISQWVCG
VLMAVAWVGSCVHSLVQIFLALSLPFCGPNVINHCFCDLQPLLKQACSETYVVNLLLVSNSGAICAVSYV
MLIFSYVIFLHSLRNHSAEVIKKALSTCVSHIIVVILFFGPCIFMYTCPATVFPMDKMIAVFYTVGTSFL
NPVIYTLKNTEVKSAMRKLWSKKLITDDKR

The NOV5 nucleic acids and proteins of the invention are useful in potential therapeutic applications implicated in used in the treatment of infections such as bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The polypeptides can be used as immunogens to produce antibodies specific for the invention, and as vaccines. They can also be used to screen for potential agonist and antagonist compounds. For example, a cDNA encoding the GPCR-like protein may be useful in gene therapy, and the GPCR-like protein may be useful when administered to a subject in need thereof. By way of nonlimiting example, the compositions of the present invention will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The novel nucleic acid encoding GPCR-like protein, and the GPCR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

In all BLAST alignments described herein, the “E-value” or “Expect” value is a numeric indication of the probability that the aligned sequences could have achieved their similarity to the BLAST query sequence by chance alone, within the database that was searched. All BLAST analyses described herein were performed using the NOV5 amino acid sequence.

A BLASTX search was performed against public protein databases. The amino acid sequence of the protein of the invention was found to have 157 of 302 amino acid residues (51%) identical to, and 215 of 302 residues (70%) positive with, the 308 amino acid odorant receptor 16 from Mus musculus (gi|11496249|ref|NP_{—}067343.1|, E=8e-77). The disclosed protein is also similar to the olfactory proteins disclosed in Table 5C.

TABLE 5C
BLAST results for NOV5.

| | | | | Posi- | |
|---|---|---|---|---|---|
| Gene Index/ | Protein/ | Length | Identity | tives | |
| Identifier | Organism | (aa) | (%) | (%) | Expect |
| Gi\|11496249\|ref\| | Odorant | 308 | 157/302 | 215/302 | 8e-77 |
| NP_067343.1; | receptor 16 | | (51%) | (70%) | |
| Gi\|6178008\|dbj\| | Mus musculus; | | | | |
| BAA86126.1 | Odorant | | | | |
| (AB030895) | receptor | | | | |
| | MOR18 | | | | |
| | Mus musculus | | | | |
| Gi\|11464995\|ref\| | Gene for | 302 | 150/302 | 206/302 | 8e-73 |
| NP_065261.1\|; | odorant | | (49%) | (67%) | |
| Gi\|6178010\|dbj\| | receptor A16 | | | | |
| BAA86127.1 | Mus musculus; | | | | |
| (AB030896) | Odorant | | | | |
| | receptor A16 | | | | |
| | Mus musculus | | | | |
| Gi\|423702\|pir∥ | Olfactory | 307 | 159/304 | 204/304 | 3e-72 |
| S29710 | receptor OR18 | | (52%) | (66%) | |
| | Rat | | | | |
| Gi\|11464993\|ref\| | Gene for | 308 | 141/298 | 196/298 | 6e-65 |
| NP_065260.1; | odorant | | (47%) | (65%) | |
| Gi\|6178006\|dbj\| | receptor | | | | |
| BAA86125.1 | MOR83 | | | | |
| (AB030894) | Mus musculus; | | | | |
| | Odorant | | | | |
| | receptor | | | | |
| | MOR83 | | | | |
| | Mus musculus | | | | |
| Gi\|10644517\|gb\| | Odorant | 264 | 126/242 | 174/242 | 3e-59 |
| AAG21323.1\| | receptor | | (52%) | (71%) | |
| AF271050_1 | Rattus | | | | |
| (AF271050) | norvegicus | | | | |

A ClustalW analysis comparing disclosed proteins of the invention with related OR protein sequences (as determined by BLASTX analysis) is given in Table 5D, with NOV5 shown on line 1.

[image]

In the ClustalW alignment of the NOV5 protein, as well as all other ClustalW analyses described herein, the black outlined amino acid residues indicate regions of conserved sequence (i.e., regions that may be required to preserve structural or functional properties), whereas non-highlighted amino acid residues are less conserved and can potentially be mutated to a much broader extent without altering protein structure or function.

The presence of identifiable domains in NOV5, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results, e.g., for NOV3, were collected from the Conserved Domain Database (CDD) with Reverse Position Specific BLAST analyses. This BLAST analysis software samples domains found in the Smart and Pfam collections. For all successive DOMAIN sequence alignments, fully conserved single residues are indicated by black shading and “strong” semi-conserved residues are indicated by grey shading. The “strong” group of conserved amino acid residues may be any one of the following groups of amino acids: STA, NEQK, NHQK, NDEQ, QHRK, MILV, MILF, HY, FYW.

The region from amino acid residue 47 through 234 (SEQ ID NO:21) most probably (E=1e⁻¹²) contains a “seven transmembrane receptor (rhodopsin family) fragment” domain, aligned here with residues 9-177 of the 7tm_{—}1 entry (TM7, SEQ ID NO:39) of the Pfam database. This indicates that the NOV5 sequence has properties similar to those of other proteins known to contain this domain as well as to the 377 amino acid 7tm domain itself. Table 5E shows the results of the domain analyses of NOV5 and the TM7 protein.

[image]

[image]

The representative member of the 7 transmembrane receptor family is the D2 dopamine receptor from Bos taurus (SWISSPROT: locus D2DR_BOVIN, accession P20288; gene index 118205). The D2 receptor is an integral membrane protein and belongs to Family 1 of G-protein coupled receptors. The activity of the D2 receptor is mediated by G proteins, which inhibit adenylyl cyclase. Chio et al., Nature 343:255-269 (1990). Residues 51-427 of this 444 amino acid protein are considered to be the representative TM7 domain, shown in Table 5F.

TABLE 5F

| | |
|---|---|
| Amino Acid sequence for TM7 | (SEQ ID NO:39). |

GNVLVCMAVSREKALQTTTNYLIVSLAVADLLVATLVMPWVVYLEVVGEWKFSRIHCDIF
VTLDVMMCTASILNLCAISIDRYTAVAMPMLYNTRYSSKRRVTVMIAIVWVLSFTISCPM
LFGLNNTDQNECIIANPAFVVYSSIVSFYVPFIVTLLVYIKIYIVLRRRRKRVNTKRSSR
AFRANLKAPLKGNCTHPEDMKLCTVIMKSNGSFPVNRRRVEAARRAQELEMEMLSSTSPP
ERTRYSPIPPSHHQLTLPDPSHHGLHSTPDSPAKPEKNGHAKTVNPKIAKIFEIQSMPNG
KTRTSLKTMSRRKLSQQKEKKATQMLAIVLGVFIICWLPFFITHILNTHCDCNIPPVLYS
AFTWLGYVNSAVNPIIY

The 7 transmembrane receptor family includes a number of different proteins, including, for example, serotonin receptors, dopamine receptors, histamine receptors, andrenergic receptors, cannabinoid receptors, angiotensin II receptors, chemokine receptors, opioid receptors, G-protein coupled receptor (GPCR) proteins, olfactory receptors (OR), and the like. Some proteins and the Protein Data Base Ids/gene indexes include, for example: rhodopsin (129209); 5-hydroxytryptamine receptors; (112821, 8488960, 112805, 231454, 1168221, 398971, 112806); G protein-coupled receptors (119130, 543823, 1730143, 132206, 137159, 6136153, 416926, 1169881, 136882, 134079); gustatory receptors (544463, 462208); c-x-c chemokine receptors (416718, 128999, 416802, 548703, 1352335); opsins (129193, 129197, 129203); and olfactory receptor-like proteins (129091, 1171893, 400672, 548417).

The nucleic acids and proteins of NOV5 are useful in potential therapeutic applications implicated in various NOV- or olfactory receptor (OR)-related pathologies and/or disorders. For example, a cDNA encoding the G-protein coupled receptor-like protein may be useful in gene therapy, and the G-protein coupled receptor-like protein may be useful when administered to a subject in need thereof. The novel nucleic acid encoding NOV5 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. The NOVX nucleic acids and proteins are useful in potential diagnostic and therapeutic applications implicated in various diseases and disorders described below and/or other pathologies. For example, the compositions of the present invention will have efficacy for treatment of patients suffering from: cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, fertility, hemophilia, hypercoagulation, idiopathic thrombocytopenic purpura, immunodeficiencies, graft versus host disease, bronchial asthma, and other diseases, disorders and conditions of the like. By way of nonlimiting example, the compositions of the present invention will have efficacy for treatment of patients suffering from neoplasm, adenocarcinoma, lymphoma, prostate cancer, uterus cancer, immune response, AIDS, asthma, Crohn's disease, multiple sclerosis, and Albright Hereditary Ostoeodystrophy. Additional NOV-related diseases and disorders are mentioned throughout the Specification.

Further, the protein similarity information, expression pattern, and map location for NOV5 suggests that NOV5 may have important structural and/or physiological functions characteristic of the NOV family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immuno-specifically to the novel NOV5 substances for use in therapeutic or diagnostic methods. These antibodies may be generated according to methods known in the art, using prediction from hydrophobicity charts, as described in the “Anti-NOVX Antibodies” section below.

NOV6

A novel nucleic acid was identified on chromosome 11 by TblastN using CuraGen Corporation's sequence file for GPCR probe or homolog, run against the Genomic Daily Files made available by GenBank. The nucleic acid was further predicted by the program GenScan™, including selection of exons. These were further modified by means of similarities using BLAST searches. The sequences were then manually corrected for apparent inconsistencies, thereby obtaining the sequences encoding the full-length protein.

A disclosed NOV6 nucleic acid of 1000 nucleotides (95-h-6-C, SEQ ID NO:22) encoding a novel NOV6 olfactory receptor-like protein is shown in Table 6A. An open reading frame (ORF) was identified beginning with an ATG initiation codon at nucleotides 9-11 and ending with a TGA codon at nucleotides 987-989. Putative untranslated regions upstream from the initiation codon and downstream from the termination codon are underlined in Table 6A, and the start and stop codons are in bold letters.

TABLE 6A

| | |
|---|---|
| NOV6 Nucleotide Sequence | (SEQ ID NO:22). |

TATAAATTATGTCATTTCAGGTGACTTATATGTTCTATCTACACTGGACCATGGAAAAAAGCAATAATAG
CACTTTGTTTATTCTCTTGGGGTTTTCCCAAAATAAGAACATTGAAGTCCTCTGCTTTGTATTATTTTTG
TTTTGCTACATTGCTATTTGGATGGGAAACTTACTCATAATGATTTCTATCACGTGCACCCAGCTCATTC
ACCAACCCATGTATTTCTTCCTCAATTACCTCTCACTCTCCGACCTTTGCTACACATCCACAGTGACCCC
CAAATTAATGGTTGACTTACTGGCAGAAAGAAAGACCATTTCCTATAATAACTGTATGATACAACTCTTT
ACCACCCATTTTTTTGGAGGCATAGAGATCTTCATTCTCACAGGGATGGCCTATGACCGCTATGTGGCCA
TTTGCAAGCCCCTGCACTACACCATTATTATGAGCAGGCAAAAGTGTAACACAATCATCATAGTTTGTTG
TACTGGGGGATTTATACATTCTGCCAGTCAGTTTCTTCTCACCATCTTTGTACCATTTTGTGGCCCAAAT
GAGATAGATCACTACTTCTGTGATGTGTATCCTTTGCTGAAATTGGCCTGTTCTAATATACACATGATAG
GTCTCTTAGTCATTGCTAATTCAGGCTTAATTGCTTTGGTGACATTTGTTGTCTTGTTGTTGTCTTATGT
TTTTATATTGTATACCATCAGAGCATACTCTGCAGAGAGACGCAGCAAAGCTCTTGCCACTTGTAGTTCT
CATGTAATTGTTGTGGTCCTGTTTTTTGCTCCTGCATTGTTCATTTACATTAGACCGGTCACAACATTCT
CAGAAGATAAAGTGTTTGCCCTTTTTTATACCATCATTGCTCCCATGTTCAACCCTCTCATATACACGCT
GAGAAACACAGAGATGAAGAACGCCATGAGGAAAGTGTGGTGTTGTCAAATACTCCTGAAAAGAAATCAA
CTTTTCTGAATTGTTTCTGC

A disclosed NOV6 polypeptide (SEQ ID NO:23) encoded by SEQ ID NO:22 is 326 amino acid residues and is presented using the one-letter code in Table 6B. The disclosed NOV6 protein was analyzed for signal peptide prediction and cellular localization. SignalP results predict that NOV6 is cleaved between position 64 and 65 of SEQ ID NO:23, i.e., at the slash in the amino acid sequence TCT-QL. Psort and Hydropathy profiles also predict that NOV6 contains a signal peptide and is localized in the plasma membrane (certainty=0.6000).

TABLE 6B

| | |
|---|---|
| Encoded NOV6 protein sequence | (SEQ ID NO:23). |

MSFQVTYMFYLHWTMEKSNNSTLFILLGFSQNKNIEVLCFVLFLFCYIAIWMGNLLIMISITCTQLIHQP
MYFFLNYLSLSDLCYTSTVTPKLMVDLLAERKTISYNNCMIQLFTTHFFGGIEIFILTGMAYDRYVAICK
PLHYTIIMSRQKCNTIIIVCCTGGFIHSASQFLLTIFVPFCGPNEIDHYFCDVYPLLKLACSNIHMIGLL
VIANSGLIALVTFVVLLLSYVFILYTIRAYSAERRSKALATCSSHVIVVVLFFAPALFIYIRPVTTFSED
KVFALFYTIIAPMFNPLIYTLRNTEMKNAMRKVWCCQILLKRNQLF

The NOV6 nucleic acids and proteins of the invention are useful in potential therapeutic applications implicated in used in the treatment of infections such as bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The polypeptides can be used as immunogens to produce antibodies specific for the invention, and as vaccines. They can also be used to screen for potential agonist and antagonist compounds. For example, a cDNA encoding the GPCR-like protein may be useful in gene therapy, and the GPCR-like protein may be useful when administered to a subject in need thereof. By way of nonlimiting example, the compositions of the present invention will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The novel nucleic acid encoding GPCR-like protein, and the GPCR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

In all BLAST alignments described herein, the “E-value” or “Expect” value is a numeric indication of the probability that the aligned sequences could have achieved their similarity to the BLAST query sequence by chance alone, within the database that was searched. All BLAST analyses described herein were performed using the NOV6 amino acid sequence.

A BLASTX search was performed against public protein databases. The amino acid sequence of the protein of the invention was found to have 163 of 300 amino acid residues (54%) identical to, and 216 of 300 residues (71%) positive with, the 308 amino acid odorant receptor 16 from Mus musculus (gi|11496249|ref|NP_{—}067343.11, E=1e-77). The disclosed protein is also similar to the olfactory proteins disclosed in Table 6C.

TABLE 6C
BLAST results for NOV6.

| | | | | Posi- | |
|---|---|---|---|---|---|
| Gene Index/ | Protein/ | Length | Identity | tives | |
| Identifier | Organism | (aa) | (%) | (%) | Expect |
| Gi\|11496249\|ref\| | Odorant | 308 | 163/300 | 216/300 | 1e-77 |
| NP_067343.1; | receptor 16 | | (54%) | (71%) | |
| Gi\|6178008\|dbj\| | Mus musculus; | | | | |
| BAA86126.1 | Odorant | | | | |
| (AB030895) | receptor | | | | |
| | MOR18 | | | | |
| | Mus musculus | | | | |
| Gi\|11464995\|ref\| | Gene for | 302 | 161/300 | 216/300 | 6e-77 |
| NP_065261.1\|; | odorant | (53%) | (71%) | | |
| Gi\|6178010\|dbj\| | receptor A16 | | | | |
| BAA86127.1 | Mus musculus; | | | | |
| (AB030896) | Odorant | | | | |
| | receptor A16 | | | | |
| | Mus musculus | | | | |
| Gi\|423702\|pir∥ | Olfactory | 307 | 161/301 | 213/301 | 1e-73 |
| S29710 | receptor OR18 | | (53%) | (70%) | |
| | Rat | | | | |
| Gi\|11464993\|ref\| | Gene for | 308 | 152/290 | 209/290 | 9e-71 |
| NP_065260.1; | odorant | | (52%) | (71%) | |
| Gi\|6178006\|dbj\| | receptor | | | | |
| BAA86125.1 | MOR83 | | | | |
| (AB030894) | Mus musculus; | | | | |
| | Odorant | | | | |
| | receptor | | | | |
| | MOR83 | | | | |
| | Mus musculus | | | | |
| Gi\|10644519\|gb\| | Odorant | 264 | 146/264 | 203/264 | 7e-69 |
| AAG21324.1\| | receptor | | (55%) | (76%) | |
| AF271051_1 | Mus musculus | | | | |
| (AF271051) | | | | | |

A ClustalW analysis comparing disclosed proteins of the invention with related OR protein sequences (as determined by BLASTX analysis) is given in Table 6D, with NOV6 shown on line 1.

[image]

In the ClustalW alignment of the NOV6 protein, as well as all other ClustalW analyses described herein, the black outlined amino acid residues indicate regions of conserved sequence (i.e., regions that may be required to preserve structural or functional properties), whereas non-highlighted amino acid residues are less conserved and can potentially be mutated to a much broader extent without altering protein structure or function.

The presence of identifiable domains in NOV6, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro. DOMAIN results, e.g., for NOV3, were collected from the Conserved Domain Database (CDD) with Reverse Position Specific BLAST analyses. This BLAST analysis software samples domains found in the Smart and Pfam collections. For all successive DOMAIN sequence alignments, fully conserved single residues are indicated by black shading and “strong” semi-conserved residues are indicated by grey shading. The “strong” group of conserved amino acid residues may be any one of the following groups of amino acids: STA, NEQK, NHQK, NDEQ, QHRK, MILV, MILF, HY, FYW.

The region from acid residue 53 through 213 (SEQ ID NO:23) most probably (E=3e⁻¹⁶) contains a “seven transmembrane receptor (rhodopsin family) fragment” domain, aligned here with residues 1-156 of the 7tm_{—}1 entry (TM7, SEQ ID NO:39) of the Pfam database. This indicates that the NOV6 sequence has properties similar to those of other proteins known to contain this domain as well as to the 377 amino acid 7tm domain itself. Table 6E shows the results of the domain analysis of NOV6 and the TM7 protein.

[image]

[image]

The representative member of the 7 transmembrane receptor family is the D2 dopamine receptor from Bos taurus (SWISSPROT: locus D2DR_BOVIN, accession P20288; gene index 118205). The D2 receptor is an integral membrane protein and belongs to Family 1 of G-protein coupled receptors. The activity of the D2 receptor is mediated by G proteins, which inhibit adenylyl cyclase. Chio et al., Nature 343:255-269 (1990). Residues 51-427 of this 444 amino acid protein are considered to be the representative TM7 domain, shown in Table 6F.

TABLE 6F
Amino Acid sequence for TM7 (SEQ ID NO:39)

GNVLVCMAVSREKALQTTTNYLIVSLAVADLLVATLVMPWVVYLEVVGEWKFSRIHCDIF
VTLDVMMCTASILNLCAISIDRYTAVAMPMLYNTRYSSKRRVTVMIAIVWVLSFTISCPM
LFGLNNTDQNECIIANPAFVVYSSIVSFYVPFIVTLLVYIKIYIVLRRRRKRVNTKRSSR
AFRANLKAPLKGNCTHPEDMKLCTVIMKSNGSFPVNRRRVEAARRAQELEMEMLSSTSPP
ERTRYSPIPPSHHQLTLPDPSHHGLHSTPDSPAKPEKNGHAKTVNPKIAKIFEIQSMPNG
KTRTSLKTMSRRKLSQQKEKKATQMLAIVLGVFIICWLPFFITHILNIHCDCNIPPVLYS
AFTWLGYVNSAVNPIIY

The 7 transmembrane receptor family includes a number of different proteins, including, for example, serotonin receptors, dopamine receptors, histamine receptors, andrenergic receptors, cannabinoid receptors, angiotensin II receptors, chemokine receptors, opioid receptors, G-protein coupled receptor (GPCR) proteins, olfactory receptors (OR), and the like. Some proteins and the Protein Data Base Ids/gene indexes include, for example: rhodopsin (129209); 5-hydroxytryptamine receptors; (112821, 8488960, 112805, 231454, 1168221, 398971, 112806); G protein-coupled receptors (119130,543823, 1730143, 132206, 137159, 6136153, 416926, 1169881, 136882, 134079); gustatory receptors (544463, 462208); c-x-c chemokine receptors (416718, 128999, 416802, 548703, 1352335); opsins (129193, 129197, 129203); and olfactory receptor-like proteins (129091, 1171893, 400672, 548417).

The nucleic acids and proteins of NOV6 are useful in potential therapeutic applications implicated in various NOV- or olfactory receptor (OR)-related pathologies and/or disorders. For example, a cDNA encoding the G-protein coupled receptor-like protein may be useful in gene therapy, and the G-protein coupled receptor-like protein may be useful when administered to a subject in need thereof. The novel nucleic acid encoding NOV6 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. The NOVX nucleic acids and proteins are useful in potential diagnostic and therapeutic applications implicated in various diseases and disorders described below and/or other pathologies. For example, the compositions of the present invention will have efficacy for treatment of patients suffering from: cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, fertility, hemophilia, hypercoagulation, idiopathic thrombocytopenic purpura, immunodeficiencies, graft versus host disease, bronchial asthma, and other diseases, disorders and conditions of the like. By way of nonlimiting example, the compositions of the present invention will have efficacy for treatment of patients suffering from neoplasm, adenocarcinoma, lymphoma, prostate cancer, uterus cancer, immune response, AIDS, asthma, Crohn's disease, multiple sclerosis, and Albright Hereditary Ostoeodystrophy. Additional NOV-related diseases and disorders are mentioned throughout the Specification.

Further, the protein similarity information, expression pattern, and map location for NOV6 suggests that NOV6 may have important structural and/or physiological functions characteristic of the NOV family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immuno-specifically to the novel NOV6 substances for use in therapeutic or diagnostic methods. These antibodies may be generated according to methods known in the art, using prediction from hydrophobicity charts, as described in the “Anti-NOVX Antibodies” section below.

NOV7

A novel NOV7 nucleic acid was identified on chromosome 11 by TblastN using CuraGen Corporation's sequence file for GPCR probe or homolog, run against the Genomic Daily Files made available by GenBank. The nucleic acid was further predicted by the program GenScan™, including selection of exons. These were further modified by means of similarities using BLAST searches. The sequences were then manually corrected for apparent inconsistencies, thereby obtaining the sequences encoding the protein fragment.

A disclosed nucleic acid of 868 nucleotides (95-h-6-D, SEQ ID NO:24) encoding a novel NOV7 olfactory receptor-like protein is shown in Table 7A. An open reading frame (ORF) was identified beginning with an ATG initiation codon at nucleotides 11-13 extending through nucleotide 868. Putative untranslated regions upstream from the initiation codon are underlined in Table 7A, and the start codon is in bold letters.

TABLE 7A
NOV7 Nucleotide Sequence (SEQ ID NO:24).

GACCCATTCCATGGAAAAAATAAACAACGTAACTGAATTCATTTTCTGGGGTCTTTCTCAGAGCCCAGAG
ATTGAGAAAGTTTGTTTTGTGGTGTTTTCTTTCTTCTACATAATCATTCTTCTGGGAAATCTCCTCATCA
TGCTGACAGTTTGCCTGAGCAACCTGTTTAAGTCACCCATGTATTTCTTTCTCAGCTTCTTGTCTTTTGT
GGACATTTGTTACTCTTCAGTCACAGCTCCCAAGATGATTGTTGACCTGTTAGCAAAGGACAAAACCATC
TCCTATGTGGGGTGCATGTTGCAACTGCTTGGAGTACATTTCTTTGGTTGCACTGAGATCTTCATCCTTA
CTGTAATGGCCTATGATCGTTATGTGGCTATCTGTAAACCCCTACATTATATGACCATCATGAACCGGGA
GACATGCAATAAAATGTTATTAGGGACGTGGGTAGGTGGGTTCTTACACTCCATTATCCAAGTGGCTCTG
GTAGTCCAACTACCCTTTTGTGGACCCAATGAGATAGATCACTACTTTTGTGATGTTCACCCTGTGTTGA
AACTTGCCTGCACAGAAACATACATTGTTGGTGTTGTTGTGACAGCCAACAGTGGTACCATTGCTCTGGG
GAGTTTTGTTATCTTGCTAATCTCCTACAGCATCATCCTAGTTTCCCTGAGAAAGCAGTCAGCAGAAGGC
AGGCGCAAAGCCCTCTCCACCTGTGGCTCCCACATTGCCATGGTCGTTATCTTTTCGAGCCCCTCTACTT
TTATGTACATGCGCCCTGATACGACCTTTTCAGAGGATAAGATGGTGGCTGTATTTTACACCATTATCAC
TCCCATGTTAAATCCTCTGATTTATACA

The disclosed NOV7 polypeptide (SEQ ID NO:25) encoded by SEQ ID NO:24 is 286 amino acid residues and is presented using the one-letter code in Table 7B. The disclosed NOV7 protein was analyzed for signal peptide prediction and cellular localization. SignalP results predict that NOV7 is cleaved between position 50 and 51 of SEQ ID NO:25, i.e., at the slash in the amino acid sequence CLS-NL. Psort and Hydropathy profiles also predict that NOV7 contains a signal peptide and is localized in the plasma membrane (certainty=0.6000).

TABLE 7B
Encoded NOV7 protein sequence (SEQ ID NO:25).

MEKINNVTEFIFWGLSQSPEIEKVCFVVFSFFYIIILLGNLLIMLTVCLSNLFKSPMYFFLSFLSFVDIC
YSSVTAPKMIVDLLAKDKTISYVGCMLQLLGVHFFGCTEIFILTVMAYDRYVAICKPLHYMTIMNRETCN
KMLLGTWVGGFLHSIIQVALVVQLPFCGPNEIDHYFCDVHPVLKLACTETYIVGVVVTANSGTIALGSFV
ILLISYSIILVSLRKQSAEGRRKALSTCGSHIAMVVIFSSPCTFMYMRPDTTFSEDKMVAVFYTIITPML
NPLIYT

The NOV7 nucleic acids and proteins of the invention are useful in potential therapeutic applications implicated in used in the treatment of infections such as bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The polypeptides can be used as immunogens to produce antibodies specific for the invention, and as vaccines. They can also be used to screen for potential agonist and antagonist compounds. For example, a cDNA encoding the GPCR-like protein may be useful in gene therapy, and the GPCR-like protein may be useful when administered to a subject in need thereof. By way of nonlimiting example, the compositions of the present invention will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The novel nucleic acid encoding GPCR-like protein, and the GPCR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

In all BLAST alignments described herein, the “E-value” or “Expect” value is a numeric indication of the probability that the aligned sequences could have achieved their similarity to the BLAST query sequence by chance alone, within the database that was searched. All BLAST analyses described herein were performed using the NOV7 amino acid sequence.

A BLASTX search was performed against public protein databases. The amino acid sequence of the protein of the invention was found to have 159 of 286 amino acid residues (55%) identical to, and 192 of 286 residues (66%) positive with, the 308 amino acid odorant receptor 16 from Mus musculus (gi|11496249|ref|NP067343.11, E=2e-78). The disclosed protein is also similar to the olfactory proteins disclosed in Table 7C.

TABLE 7C
BLAST results for NOV7.

| | | | | Posi- | |
|---|---|---|---|---|---|
| Gene Index/ | Protein/ | Length | Identity | tives | |
| Identifier | Organism | (aa) | (%) | (%) | Expect |
| Gi\|11496249\|ref\| | Odorant | 308 | 159/286 | 192/286 | 2e-78 |
| NP_067343.1; | receptor 16 | | (55%) | (66%) | |
| Gi\|6178008\|dbj\| | Mus musculus; | | | | |
| BAA86126.1 | Odorant | | | | |
| (AB030895) | receptor | | | | |
| | MOR18 | | | | |
| | Mus musculus | | | | |
| Gi\|11464995\|ref\| | Gene for | 302 | 148/286 | 189/286 | 2e-73 |
| NP_065261.1\|; | odorant | | (51%) | (65%) | |
| Gi\|61780l0\|dbj\| | receptor A16 | | | | |
| BAA86127.1 | Mus musculus; | | | | |
| (AB030896) | Odorant | | | | |
| | receptor A16 | | | | |
| | Mus musculus | | | | |
| Gi\|11464993\|ref\| | Gene for | 308 | 146/280 | 196/280 | 2e-73 |
| NP_065260.1\|; | odorant | | (52%) | (69%) | |
| Gi\|6178006\|dbj\| | receptor | | | | |
| BAA86125.1 | MOR83 | | | | |
| (AB030894) | Mus musculus; | | | | |
| | Odorant | | | | |
| | receptor | | | | |
| | MOR83 | | | | |
| | Mus musculus | | | | |
| Gi\|10644517\| | Odorant | 264 | 150/263 | 186/263 | 6e-72 |
| GbAAG21323.1\| | receptor | | (57%) | (70%) | |
| AF271050_1 | Rattus | | | | |
| (AF271050) | norvegicus | | | | |
| Gi\|10644519\|gb\| | Odorant | 264 | 148/263 | 185/263 | 3e-71 |
| AAG21324.1\| | receptor | | (56%) | (70%) | |
| AF271051_1 | Mus musculus | | | | |
| (AF271051) | | | | | |

A ClustalW analysis comparing disclosed proteins of the invention with related OR protein sequences (as determined by BLASTX analysis) is given in Table 7D, with NOV7 shown on line 6.

[image]

In the ClustalW alignment of the NOV7 protein, as well as all other ClustalW analyses described herein, the black outlined amino acid residues indicate regions of conserved sequence (i.e., regions that may be required to preserve structural or functional properties), whereas non-highlighted amino acid residues are less conserved and can potentially be mutated to a much broader extent without altering protein structure or function.

The presence of identifiable domains in NOV7, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results, e.g., for NOV3, were collected from the Conserved Domain Database (CDD) with Reverse Position Specific BLAST analyses. This BLAST analysis software samples domains found in the Smart and Pfam collections. For all successive DOMAIN sequence alignments, fully conserved single residues are indicated by black shading and “strong” semi-conserved residues are indicated by grey shading. The “strong” group of conserved amino acid residues may be any one of the following groups of amino acids: STA, NEQK, NHQK, NDEQ, QHRK, MILV, MILF, HY, FYW.

The region from amino acid residue 46 through 197 (SEQ ID NO:25) most probably (E=4e⁻²⁰) contains a “seven transmembrane receptor (rhodopsin family) fragment” domain, aligned here with residues 8-154 of the 7tm_{—}1 entry (TM7, SEQ ID NO:39) of the Pfam database. This indicates that the NOV6 sequence has properties similar to those of other proteins known to contain this domain as well as to the 377 amino acid 7tm domain itself. Table 7E shows the results of the domain analysis of NOV7 and the TM7 protein.

[image]

[image]

The representative member of the 7 transmembrane receptor family is the D2 dopamine receptor from Bos taurus (SWISSPROT: locus D2DR_BOVIN, accession P20288; gene index 118205). The D2 receptor is an integral membrane protein and belongs to Family 1 of G-protein coupled receptors. The activity of the D2 receptor is mediated by G proteins, which inhibit adenylyl cyclase. Chio et al., Nature 343:255-269 (1990). Residues 51-427 of this 444 amino acid protein are considered to be the representative TM7 domain, shown in Table 7F.

TABLE 7F
Amino Acid sequence for TM7 (SEQ ID NO:39).

GNVLVCMAVSREKALQTTTNYLIVSLAVADLLVATLVMPWVVYLEVVGEWKFSRIHCDIF
VTLDVMMCTASILNLCAISIDRYTAVAMPMLYNTRYSSKRRVTVMIAIVWVLSFTISCPM
LFGLNNTDQNECIIANPAFVVYSSIVSFYVPFIVTLLVYIKIYIVLRRRRKRVNTKRSSR
AFRANLKAPLKGNCTHPEDMKLCTVIMKSNGSFPVNRRRVEAARRAQELEMEMLSSTSPP
ERTRYSPIPPSHHQLTLPDPSHHGLHSTPDSPAKPEKNGHAKTVNPKIAKIFEIQSMPNG
KTRTSLKTMSRRKLSQQKEKKATQMLAIVLGVFIICWLPFFITHILNIHCDCNIPPVLYS
AFTWLGYVNSAVNPIIY

The 7 transmembrane receptor family includes a number of different proteins, including, for example, serotonin receptors, dopamine receptors, histamine receptors, andrenergic receptors, cannabinoid receptors, angiotensin II receptors, chemokine receptors, opioid receptors, G-protein coupled receptor (GPCR) proteins, olfactory receptors (OR), and the like. Some proteins and the Protein Data Base Ids/gene indexes include, for example: rhodopsin (129209); 5-hydroxytryptamine receptors; (112821, 8488960, 112805, 231454, 1168221, 398971, 112806); G protein-coupled receptors (119130, 543823, 1730143, 132206, 137159, 6136153, 416926, 1169881, 136882, 134079); gustatory receptors (544463, 462208); c-x-c chemokine receptors (416718, 128999, 416802, 548703, 1352335); opsins (129193, 129197, 129203); and olfactory receptor-like proteins (129091, 1171893, 400672, 548417).

The nucleic acids and proteins of NOV7 are useful in potential therapeutic applications implicated in various NOV- or olfactory receptor (OR)-related pathologies and/or disorders. For example, a cDNA encoding the G-protein coupled receptor-like protein may be useful in gene therapy, and the G-protein coupled receptor-like protein may be useful when administered to a subject in need thereof. The novel nucleic acid encoding NOV7 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. The NOVX nucleic acids and proteins are useful in potential diagnostic and therapeutic applications implicated in various diseases and disorders described below and/or other pathologies. For example, the compositions of the present invention will have efficacy for treatment of patients suffering from: cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, fertility, hemophilia, hypercoagulation, idiopathic thrombocytopenic purpura, immunodeficiencies, graft versus host disease, bronchial asthma, and other diseases, disorders and conditions of the like. By way of nonlimiting example, the compositions of the present invention will have efficacy for treatment of patients suffering from neoplasm, adenocarcinoma, lymphoma, prostate cancer, uterus cancer, immune response, AIDS, asthma, Crohn's disease, multiple sclerosis, and Albright Hereditary Ostoeodystrophy. Additional NOV-related diseases and disorders are mentioned throughout the Specification.

Further, the protein similarity information, expression pattern, and map location for NOV7 suggests that NOV7 may have important structural and/or physiological functions characteristic of the NOV family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immuno-specifically to the novel NOV7 substances for use in therapeutic or diagnostic methods. These antibodies may be generated according to methods known in the art, using prediction from hydrophobicity charts as described in the “Anti-NOVX Antibodies” section below.

NOV8

A novel NOV8 nucleic acid was identified on chromosome 11 by TblastN using CuraGen Corporation's sequence file for GPCR probe or homolog, run against the Genomic Daily Files made available by GenBank. The nucleic acid was further predicted by the program GenScan™, including selection of exons. These were further modified by means of similarities using BLAST searches. The sequences were then manually corrected for apparent inconsistencies, thereby obtaining the sequences encoding the full-length protein.

A disclosed nucleic acid of 948 nucleotides (95-h-6-E, SEQ ID NO:26) encoding a novel NOV8 olfactory receptor-like protein is shown in Table 8A. An open reading frame (ORF) was identified beginning with an ATG initiation codon at nucleotides 10-12 and ending with a TAA codon at nucleotides 943-945. Putative untranslated regions upstream from the initiation codon and downstream from the stop codon are underlined in Table 8A, and the start and stop codons are in bold letters.

TABLE 8A
NOV8 Nucleotide Sequence (SEQ ID NO:26).

ACTCTCTGAATGGATCACCACATGCCTCCCAACAATGTGACTGAATTCATTCTCTTGGGGCTCACACAGA
ATCCACACTTGCAGAAAATACTCTTTATTGTATTTTTATTTATTTTTCTATTTACCATGCTGGCCAATCT
GTTCATTGTCATCACCATCTCCTGTAGCCCCACACTTTCATCACCCATGTACTTCTTTCTCACTTACTTA
TCCTTTATAGATGCCTCCTACACCTCTGTCACAACCCCCAAAATGATCACCGACCTGCTCTACCAGAGGA
GAACTATTTCCTTGGCTGGCTGCCTGACTCAGCTCTTTGTGGAGCACTTGCTGGGAGGCTCAGAGATCAT
CCTCCTTATTGTCATGGCCTATGACCGCTACGTGGCCATCTGCAAGCCCCTGCACTACACAACCATTATG
CAACAAGGGATCTGCCACCTTCTGGTGGTGATAGCCTGGATTGGAGGCATCCTGCATGCCACTGTGCAGA
TTCTTTTCATGACCGACTTGCCCTTCTGTGGTCCCAATGTCATTGACCACTTTATGTGTGATCTCTTCCC
ATTGTTGAAACTTGCCTGCAGAGACACCTACAGACTTGGGATGCTGGTGGCAGCCAACAGTGGAGCCATG
TGCTTGCTCATCTTTTCCCTGCTCGTCATCTCCTACATAGTCATCCTGAGCTCCCTGAAATCCTATAGCT
CTGAAGGACAGCGCAAAGCCCTCTCCACCTGTGGCTCCCACTTTACTGTCGTTGTACTCTTTTTTGTGCC
TTGCATATTCACCTACATGCATCCTGTGGTCACCTACTCTGTGGACAAGTTGGTGACTGTGTTCTTTGCA
ATCCTCACTCCCATGTTAAATCCTATAATTTACACTGTGAGAAACACAGAGGTAAAAAATGTCGTGAGGA
GTTTGTTGAGGAAAAGAGTAACAGTTTATGCATAATGG

The disclosed NOV8 polypeptide (SEQ ID NO:27) encoded by SEQ ID NO:26 is 311 amino acid residues and is presented using the one-letter code in Table 8B. The disclosed NOV8 protein was analyzed for signal peptide prediction and cellular localization. SignalP results predict that NOV8 is cleaved between position 42 and 43 of SEQ ID NO:27, ie., at the slash in the amino acid sequence MLA-NL. Psort and Hydropathy profiles also predict that NOV8 contains a signal peptide and is localized in the plasma membrane (certainty=0.6000).

TABLE 8B
Encoded NOV8 protein sequence (SEQ ID NO:27).

MDHHMPPNNVTEFILLGLTQNPHLQKILFIVFLFIFLFTMLANLFIVITISCSPTLSSPMYFFLTYLSFI
DASYTSVTTPKMITDLLYQRRTISLAGCLTQLFVEHLLGGSEIILLIVMAYDRYVAICKPLHYTTIMQQG
ICHLLVVIAWIGGILHATVQILFMTDLPFCGPNVIDHFMCDLFPLLKLACRDTYRLGMLVAANSGAMCLL
IFSLLVISYIVILSSLKSYSSEGQRKALSTCGSHFTVVVLFFVPCIFTYMHPVVTYSVDKLVTVFFAILT
PMLNPIIYTVRNTEVKNVVRSLLRKRVTVYA

The NOV8 nucleic acids and proteins of the invention are useful in potential therapeutic applications implicated in used in the treatment of infections such as bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The polypeptides can be used as immunogens to produce antibodies specific for the invention, and as vaccines. They can also be used to screen for potential agonist and antagonist compounds. For example, a cDNA encoding the GPCR-like protein may be useful in gene therapy, and the GPCR-like protein may be useful when administered to a subject in need thereof. By way of nonlimiting example, the compositions of the present invention will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The novel nucleic acid encoding GPCR-like protein, and the GPCR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

In all BLAST alignments described herein, the “E-value” or “Expect” value is a numeric indication of the probability that the aligned sequences could have achieved their similarity to the BLAST query sequence by chance alone, within the database that was searched. All BLAST analyses described herein were performed using the NOV8 amino acid sequence.

A BLASTX search was performed against public protein databases. The amino acid sequence of the protein of the invention was found to have 189 of 299 amino acid residues (63%) identical to, and 234 of 299 residues (74%) positive with, the 302 amino acid gene for odorant receptor A16 from Mus musculus (gi|11464995|ref|NP065261.1|, E=2e-96). The disclosed protein is also similar to the olfactory proteins disclosed in Table 8C.

TABLE 8C
BLAST results for NOV8.

| | | | | Posi- | |
|---|---|---|---|---|---|
| Gene Index/ | Protein/ | Length | Identity | tives | |
| Identifier | Organism | (aa) | (%) | (%) | Expect |
| Gi\|11464995\|ref\| | Gene for | 302 | 189/299 | 234/299 | 2e-96 |
| NP_065261.1\|; | odorant | | (63%) | (78%) | |
| Gi\|6178010\|dbj\| | receptor A16 | | | | |
| BAA86127.1 | Mus musculus; | | | | |
| (AB030896) | Odorant | | | | |
| | receptor A16 | | | | |
| | Mus musculus | | | | |
| Gi\|11496249\|ref\| | Odorant | 308 | 180/305 | 230/305 | 7e-89 |
| NP_067343.1; | receptor 16 | | (59%) | (75%) | |
| Gi\|6178008\|dbj\| | Mus musculus; | | | | |
| BAA86126.1 | Odorant | | | | |
| (AB030895) | receptor | | | | |
| | MOR18 | | | | |
| | Mus musculus | | | | |
| Gi\|423702\|pir∥ | Olfactory | 307 | 168/295 | 223/295 | 1e-83 |
| S29710 | receptor | | (56%) | (74%) | |
| | OR18 Rat | | | | |
| Gi\|11464993\|ref\| | Gene for | 308 | 156/298 | 206/298 | 1e-73 |
| NP_065260.1\|; | odorant | | (52%) | (68%) | |
| Gi\|6178006\|dbj\| | receptor | | | | |
| BAA86125.1 | MOR83 | | | | |
| (AB030894) | Mus musculus; | | | | |
| | Odorant | | | | |
| | receptor | | | | |
| | MOR83 | | | | |
| | Mus musculus | | | | |
| Gi\|3983372\|gb\| | Olfactory | 220 | 147/220 | 176/220 | 2e-70 |
| AAD13314.1\| | receptor C3 | | (66%) | (79%) | |
| (AF102522) | Mus musculus | | | | |

A ClustalW analysis comparing disclosed proteins of the invention with related OR protein sequences (as determined by BLASTX analysis) is given in Table 8D, with NOV8 shown on line 1.

[image]

[image]

In the ClustalW alignment of the NOV8 protein, as well as all other ClustalW analyses described herein, the black outlined amino acid residues indicate regions of conserved sequence (i.e., regions that may be required to preserve structural or functional properties), whereas non-highlighted amino acid residues are less conserved and can potentially be mutated to a much broader extent without altering protein structure or function.

The presence of identifiable domains in NOV8, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results, e.g., for NOV8, were collected from the Conserved Domain Database (CDD) with Reverse Position Specific BLAST analyses. This BLAST analysis software samples domains found in the Smart and Pfam collections. For all successive DOMAIN sequence alignments, fully conserved single residues are indicated by black shading and “strong” semi-conserved residues are indicated by grey shading. The “strong” group of conserved amino acid residues may be any one of the following groups of amino acids: STA, NEQK, NHQK, NDEQ, QHRK, MILV, MILF, HY, FYW.

The region from amino acid residue 43 through 237 (SEQ ID NO:27) most probably (E=1e⁻¹⁶) contains a “seven transmembrane receptor (rhodopsin family) fragment” domain, aligned here with residues 2-181 of the 7tm_{—}1 entry (TM7, SEQ ID NO:39) of the Pfam database. This indicates that the NOV8 sequence has properties similar to those of other proteins known to contain this domain as well as to the 377 amino acid 7tm domain itself. NOV8 also has identity to another region of the TM7 protein. The region from amino acid residue 224 through 288 (of SEQ ID NO:27) aligns with amino acid residues 310-377 of TM7 (E=9e-04). Table 8E shows the results of the domain analysis of NOV8 and the TM7 protein.

[image]

The representative member of the 7 transmembrane receptor family is the D2 dopamine receptor from Bos taurus (SWISSPROT: locus D2DR_BOVIN, accession P20288; gene index 118205). The D2 receptor is an integral membrane protein and belongs to Family 1 of G-protein coupled receptors. The activity of the D2 receptor is mediated by G proteins, which inhibit adenylyl cyclase. Chio et al., Nature 343:255-269 (1990). Residues 51-427 of this 444 amino acid protein are considered to be the representative TM7 domain, shown in Table 8F.

TABLE 8F
Amino Acid sequence for TM7 (SEQ ID NO:39)

GNVLVCMAVSREKALQTTTNYLIVSLAVADLLVATLVMPWVVYLEVVGEWKFSRIHCDIF
VTLDVMMCTASILNLCAISIDRYTAVAMPMLYNTRYSSKRRVTVMIAIVWVLSFTISCPM
LFGLNNTDQNECIIANPAFVVYSSIVSFYVPFIVTLLVYIKIYIVLRRRRKRVNTKRSSR
AFRANLKAPLKGNCTHPEDMKLCTVIMKSNGSFPVNRRRVEAARRAQELEMEMLSSTSPP
ERTRYSPIPPSHHQLTLPDPSHHGLHSTPDSPAKPEKNGHAKTVNPKIAKIFEIQSMPNG
KTRTSLKTMSRRKLSQQKEKKATQMLAIVLGVFIICWLPFFITHILNIHCDCNIPPVLYS
AFTWLGYVNSAVNPIIY

The 7 transmembrane receptor family includes a number of different proteins, including, for example, serotonin receptors, dopamine receptors, histamine receptors, andrenergic receptors, cannabinoid receptors, angiotensin II receptors, chemokine receptors, opioid receptors, G-protein coupled receptor (GPCR) proteins, olfactory receptors (OR), and the like. Some proteins and the Protein Data Base Ids/gene indexes include, for example: rhodopsin (129209); 5-hydroxytryptamine receptors; (112821, 8488960, 112805, 231454, 1168221, 398971, 112806); G protein-coupled receptors (119130, 543823, 1730143, 132206, 137159, 6136153, 416926, 1169881, 136882, 134079); gustatory receptors (544463, 462208); c-x-c chemokine receptors (416718, 128999, 416802, 548703, 1352335); opsins (129193, 129197, 129203); and olfactory receptor-like proteins (129091, 1171893, 400672, 548417).

The nucleic acids and proteins of NOV8 are useful in potential therapeutic applications implicated in various NOV- or olfactory receptor (OR)-related pathologies and/or disorders. For example, a cDNA encoding the G-protein coupled receptor-like protein may be useful in gene therapy, and the G-protein coupled receptor-like protein may be useful when administered to a subject in need thereof. The novel nucleic acid encoding NOV8 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. The NOVX nucleic acids and proteins are useful in potential diagnostic and therapeutic applications implicated in various diseases and disorders described below and/or other pathologies. For example, the compositions of the present invention will have efficacy for treatment of patients suffering from: cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, fertility, hemophilia, hypercoagulation, idiopathic thrombocytopenic purpura, immunodeficiencies, graft versus host disease, bronchial asthma, and other diseases, disorders and conditions of the like. By way of nonlimiting example, the compositions of the present invention will have efficacy for treatment of patients suffering from neoplasm, adenocarcinoma, lymphoma, prostate cancer, uterus cancer, immune response, AIDS, asthma, Crohn's disease, multiple sclerosis, and Albright Hereditary Ostoeodystrophy. Additional NOV-related diseases and disorders are mentioned throughout the Specification.

Further, the protein similarity information, expression pattern, and map location for NOV8 suggests that NOV8 may have important structural and/or physiological functions characteristic of the NOV family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immuno-specifically to the novel NOV8 substances for use in therapeutic or diagnostic methods. These antibodies may be generated according to methods known in the art, using prediction from hydrophobicity charts, as described in the “Anti-NOVX Antibodies” section below.

NOV9

A novel NOV9 nucleic acid was identified on chromosome 11 by TblastN using CuraGen Corporation's sequence file for GPCR probe or homolog, run against the Genomic Daily Files made available by GenBank. The nucleic acid was further predicted by the program GenScan™, including selection of exons. These were further modified by means of similarities using BLAST searches. The sequences were then manually corrected for apparent inconsistencies, thereby obtaining the sequences encoding the full-length protein.

A disclosed NOV nucleic acid of 944 nucleotides (95-h-6-SEQ ID NO:28) encoding a novel NOV9 olfactory receptor-like protein is shown in Table 9A. An open reading frame (ORF) was identified beginning with an ATG initiation codon at nucleotides 7-9 and ending with a TAG codon at nucleotides 934-936. Putative untranslated regions upstream from the initiation codon and downstream from the stop codon are underlined in Table 9A, and the start and stop codons are in bold letters.

TABLE 9A
NOV9 Nucleotide Sequence (SEQ ID NO:28).

TCTTAAATGGGGACTTCAAATAATGAGACTGAATTCATTCTTTTGGGCATTACAAAAAATCCAGAACTAA
GGAAAATATTCTCTGCTTTGTTTCTAGCCATGTATGTGACCACAGTGTTGGGAAATCTATTCATTGTGGT
GACTCTGGCTGCAAGTTGGAGTCTGAGATCACCTATGTACTTTTCCCTTACTTCCTTGTCTCTCATGGGT
GCCACCTACTCTTCCATCACTGCCCCTAAGATGACTGTGGACTCTTTTGAGAACACTACCATTTCCCTTG
AAGGCTGCATGACCCAGCTCTTTGCAGAGCATTTCTCTGATGGTGTAGCGATCATCCTTCTCACTGTGAT
GGTCTGTGACTGCTATGAGGCCATCAGTAAGCCCCTGCATGACACAACCATCATGAGTCCACGGGTGTGG
CTGCTGTTGGTGGTAGAAGCTTGGGTGGGGGGATTAACACATGCCACAATACAGCTTTTTTTTTTCATAT
ATCAAATACCCTTCTGTGGTCCCAATATTATTGACCATTTTATATGTGATTTGTTTCCATTGTTAAAACT
TGCTTACATGGACACCCACATGCTGGGTCTCTTAGTCATCCTCAACAGTGGGGTGATGTGTATGGCCATC
TTCCTTATCCTAATTGCATCCTACATTGTCACCCTGTACTCTCTGAAGTCTTGCAGCTCGGTAGGTCGAC
GCAACACACTTTCCACCTGTGGCTCCCACCACACAGTGGTCATCTTGTTCTTCGTGGAGTGTATTTTCTT
GTACATAAGACCTGTGGTCACTTACCCCATAGACAAGGATATGGCTATTTCCTTTACTATTGTTGCACCC
ATGTTAAATCCTCTGATCTATACCCTGAGGGGCATCAAGGTAAAAAATGCCATAAGAAAAATGTGGATGA
AACAGGGGACCCTAGGTGGTCACTAGCTTACATG

The disclosed NOV9 polypeptide (SEQ ID NO:29) encoded by SEQ ID NO:28 is 309 amino acid residues and is presented using the one-letter code in Table 9B. The disclosed NOV9 protein was analyzed for signal peptide prediction and cellular localization. SignalP results predict that NOV9 is cleaved between position 48 and 49 of SEQ ID NO:29, i.e., at the slash in the amino acid sequence TLA-AS. Psort and Hydropathy profiles also predict that NOV8 contains a signal peptide and is localized in the plasma membrane (certainty=0.6000).

TABLE 9B
Encoded NOV9 protein sequence (SEQ ID NO:29).

MGTSNNETEFILLGITKNPELRKIFSALFLAMYVTTVLGNLFIVVTLAASWSLRSPMYFSLTSLSLMGAT
YSSITAPKMTVDSFENTTISLSGCMTQLFAEHFSDGVAIILLTVMVCDCYEAISKPLHDTTIMSPRVWLL
LVVEAWVGGLTHATIQLFFFIYQIPFCGPNIIDHFICDLFPLLKLAYMDTHMLGLLVILNSGVMCMAIFL
ILIASYIVTLYSLKSCSSVGRRNTLSTCGSHHTVVILFFVECIFLYIRPVVTYPIDKDMAISFTIVAPML
NPLIYTLRGIKVKNAIRKMWMKQGTLGGH

The NOV9 nucleic acids and proteins of the invention are useful in potential therapeutic applications implicated in used in the treatment of infections such as bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The polypeptides can be used as immunogens to produce antibodies specific for the invention, and as vaccines. They can also be used to screen for potential agonist and antagonist compounds. For example, a cDNA encoding the GPCR-like protein may be useful in gene therapy, and the GPCR-like protein may be useful when administered to a subject in need thereof. By way of nonlimiting example, the compositions of the present invention will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The novel nucleic acid encoding GPCR-like protein, and the GPCR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

In all BLAST alignments described herein, the “E-value” or “Expect” value is a numeric indication of the probability that the aligned sequences could have achieved their similarity to the BLAST query sequence by chance alone, within the database that was searched. All BLAST analyses described herein were performed using the NOV9 amino acid sequence.

A BLASTX search was performed against public protein databases. The amino acid sequence of the protein of the invention was found to have 168 of 308 amino acid residues (54%) identical to, 227 of 308 residues (73%) positive, and gaps at 2 of 308 residues, with the 308 amino acid odorant receptor A16 from Mus musculus (gi|11496249|ref|NP067343.1|, E=4e-80). The disclosed protein is also similar to the olfactory proteins disclosed in Table 9C.

TABLE 9C
BLAST results for NOV9

| | | | | Posi- | | |
|---|---|---|---|---|---|---|
| Gene Index/ | Protein/ | Length | Identity | tives | Ex- | |
| Identifier | Organism | (aa) | (%) | (%) | pect | Gap |
| Gi\|11496249\|ref\| | Odorant | 308 | 168/308 | 227/308 | 4e-80 | 2/308 |
| NP_067343.1; | receptor | | (54%) | (73%) | | |
| Gi\|6178008\|dbj\| | 16 | | | | | |
| BAA86126.1 | Mus | | | | | |
| (AB030895) | musculus; | | | | | |
| | Odorant | | | | | |
| | receptor | | | | | |
| | MOR18 | | | | | |
| | Mus | | | | | |
| | musculus | | | | | |
| Gi\|423702\|pir∥ | Olfactory | 307 | 164/303 | 214/303 | 2e-74 | 2/303 |
| S29710 | receptor | | (54%) | (70%) | | |
| | OR18 Rat | | | | | |
| Gi\|11464995\|ref\| | Gene for | 302 | 159/303 | 216/303 | 4e-74 | 2/303 |
| NP_065261.1\| | odorant | | (52%) | (70%) | | |
| Gi\|6178010\|dbj\| | receptor | | | | | |
| BAA86127.1 | A16 | | | | | |
| (AB030896) | Mus | | | | | |
| | musculus; | | | | | |
| | Odorant | | | | | |
| | receptor | | | | | |
| | A16 | | | | | |
| | Mus | | | | | |
| | musculus | | | | | |
| Gi}11463993\|ref\| | Gene for | 308 | 138/302 | 192/302 | 6e-58 | 4/302 |
| NP_065260.1\| | odorant | | (45%) | (62%) | | |
| Gi\|6178006\|dbj\| | receptor | | | | | |
| BAA86125.1 | MOR83 | | | | | |
| (AB030894) | Mus | | | | | |
| | musculus; | | | | | |
| | Odorant | | | | | |
| | receptor | | | | | |
| | MOR83 | | | | | |
| | Mus | | | | | |
| | musculus | | | | | |
| Gi\|10644517\|gb\| | Odorant | 264 | 138/261 | 177/261 | 2e-56 | 2/261 |
| AAG21323.1\| | receptor | | (52%) | (66%) | | |
| AF271050_1 | Rattus | | | | | |
| (AF271050) | norvegicus | | | | | |

A ClustalW analysis comparing disclosed proteins of the invention with related OR protein sequences (as determined by BLASTX analysis) is given in Table 9D, with NOV9 shown on line 1.

[image]

[image]

In the ClustalW alignment of the NOV9 protein, as well as all other ClustalW analyses described herein, the black outlined amino acid residues indicate regions of conserved sequence (i.e., regions that may be required to preserve structural or functional properties), whereas non-highlighted amino acid residues are less conserved and can potentially be mutated to a much broader extent without altering protein structure or function.

The presence of identifiable domains in NOV9, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results, e.g., for NOV9, were collected from the Conserved Domain Database (CDD) with Reverse Position Specific BLAST analyses. This BLAST analysis software samples domains found in the Smart and Pfam collections. For all successive DOMAIN sequence alignments, fully conserved single residues are indicated by black shading and “strong” semi-conserved residues are indicated by grey shading. The “strong” group of conserved amino acid residues may be any one of the following groups of amino acids: STA, NEQK, NHQK, NDEQ, QHRK, MILV, MILF, HY, FYW.

The region from amino acid residue 39 through 199 (SEQ ID NO:29) most probably (E=7e⁻¹¹) contains a “seven transmembrane receptor (rhodopsin family) fragment” domain, aligned here with residues 1-156 of the 7tm_{—}1 entry (TM7, SEQ ID NO:39) of the Pfam database. This indicates that the NOV9 sequence has properties similar to those of other proteins known to contain this domain as well as to the 377 amino acid 7tm domain itself. Table 9E shows the results of the domain analysis of NOV9 and the TM7 protein.

[image]

The representative member of the 7 transmembrane receptor family is the D2 dopamine receptor from Bos taurus (SWISSPROT: locus D2DR_BOVIN, accession P20288; gene index 118205). The D2 receptor is an integral membrane protein and belongs to Family 1 of G-protein coupled receptors. The activity of the D2 receptor is mediated by G proteins, which inhibit adenylyl cyclase. Chio et al., Nature 343:255-269 (1990). Residues 51-427 of this 444 amino acid protein are considered to be the representative TM7 domain, shown in Table 9F.

TABLE 9F
Amino Acid sequence for TM7 (SEQ ID NO:39).

GNVLVCMAVSREKALQTTTNYLIVSLAVADLLVATLVMPWVVYLEVVGEWKFSRIHCDIF
VTLDVMMCTASILNLCAISIDRYTAVAMPMLYNTRYSSKRRVTVMIAIVWVLSFTISCPM
LFGLNNTDQNECIIANPAFVVYSSIVSFYVPFIVTLLVYIKIYIVLRRRRKRVNTKRSSR
AFRANLKAPLKGNCTHPEDMKLCTVIMKSNGSFPVNRRRVEAARRAQELEMEMLSSTSPP
ERTRYSPIPPSHHQLTLPDPSHHGLHSTPDSPAKPEKNGHAKTVNPKIAKIFEIQSMPNG
KTRTSLKTMSRRKLSQQKEKKATQMLAIVLGVFIICWLPFFITHILNIHCDCNIPPVLYS
AFTWLGYVNSAVNPIIY

The 7 transmembrane receptor family includes a number of different proteins, including, for example, serotonin receptors, dopamine receptors, histamine receptors, andrenergic receptors, cannabinoid receptors, angiotensin II receptors, chemokine receptors, opioid receptors, G-protein coupled receptor (GPCR) proteins, olfactory receptors (OR), and the like. Some proteins and the Protein Data Base Ids/gene indexes include, for example: rhodopsin (129209); 5-hydroxytryptamine receptors; (112821, 8488960, 112805, 231454, 1168221, 398971, 112806); G protein-coupled receptors (119130, 543823, 1730143, 132206, 137159, 6136153, 416926, 1169881, 136882, 134079); gustatory receptors (544463, 462208); c-x-c chemokine receptors (416718, 128999, 416802, 548703, 1352335); opsins (129193, 129197, 129203); and olfactory receptor-like proteins (129091, 1171893, 400672, 548417).

The nucleic acids and proteins of NOV9 are useful in potential therapeutic applications implicated in various NOV- or olfactory receptor (OR)-related pathologies and/or disorders. For example, a cDNA encoding the G-protein coupled receptor-like protein may be useful in gene therapy, and the G-protein coupled receptor-like protein may be useful when administered to a subject in need thereof. The novel nucleic acid encoding NOV9 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. The NOVX nucleic acids and proteins are useful in potential diagnostic and therapeutic applications implicated in various diseases and disorders described below and/or other pathologies. For example, the compositions of the present invention will have efficacy for treatment of patients suffering from: cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, fertility, hemophilia, hypercoagulation, idiopathic thrombocytopenic purpura, immunodeficiencies, graft versus host disease, bronchial asthma, and other diseases, disorders and conditions of the like. By way of nonlimiting example, the compositions of the present invention will have efficacy for treatment of patients suffering from neoplasm, adenocarcinoma, lymphoma, prostate cancer, uterus cancer, immune response, AIDS, asthma, Crohn's disease, multiple sclerosis, and Albright Hereditary Ostoeodystrophy. Additional NOV-related diseases and disorders are mentioned throughout the Specification.

Further, the protein similarity information, expression pattern, and map location for NOV9 suggests that NOV9 may have important structural and/or physiological functions characteristic of the NOV family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immuno-specifically to the novel NOV9 substances for use in therapeutic or diagnostic methods. These antibodies may be generated according to methods known in the art, using prediction from hydrophobicity charts, as described in the “Anti-NOVX Antibodies” section below.

NOV10

A novel NOV10 nucleic acid was identified on chromosome 11 by ThlastN using CuraGen Corporation's sequence file for GPCR probe or homolog, and run against the Genomic Daily Files made available by GenBank. The nucleic acid was further predicted by the program GenScan™, including selection of exons. These were further modified by means of similarities using BLAST searches. The sequences were then manually corrected for apparent inconsistencies, thereby obtaining the sequences encoding the full-length protein.

A disclosed NOV10 nucleic acid of 1003 nucleotides (AC023078 A, SEQ ID NO:30) encoding a novel NOV10 olfactory receptor-like protein is shown in Table 10A. An open reading frame (ORF) was identified beginning with an ATG initiation codon at nucleotides 5-7 and ending with a TGA codon at nucleotides 995-997. Putative untranslated regions upstream from the initiation codon and downstream from the stop codon are underlined in Table 10A, and the start and stop codons are in bold letters.

TABLE 10A
NOV10 Nucleotide Sequence (SEQ ID NO:30).

GAGCATGAATCCAACCATCCCAGCCTTGGATACAGAAATTGCACCAATTAGTGATACAGAGGAGACCCAT
CCTCATCGTTGTGGCATGGAGGTCCTGGTCCTCATAGTGCTGATCCTCATCATTGACCTGGTCGGGCTGG
CAGGAAATGCAGTCATGCTCTGGCTCCTGGGCTTCTGCATGCACAGTAACACCTTCTCTCTCTACATCCT
CAACCTGGCCAGGGCTGACTTCCTCTGCACCTGCTTCCAGATTATAACATTCATTAATTTCTTCAGTGAC
TTTGTTAGTTCTCTCTCCATCCATTTCTCTAGATTTGTCACCACGGTGTTGTTCTCCGCCTGTATTACAG
GCCTGAGCATGCTGAGCACCATCAGCACCGAGCACCGCCTGTCCGTCCTGTGGCCCATCTGGTACTGCTG
CCACTGCCCCACACACCTGTCAGCGGTCATGTGTGTCCTGCTCTGGGCCCTGTCCCTGTTGCAGAGCATC
CTGGAGTGGATGTTCTGTAGCTTCCTGTTTAGTGATGTTGACTCTGATAATTGGTGTCAAATATTAGATT
TCCTCACTGCTGTGTGGCTGATTTTTTTATCTGTGGTTCTCTGTGGGTTCACCCTGGTCCTGCTTGTCAG
GATCATATGTGGATCCCAGAAGATGCCGCTGACCAGGCTGTATGTGACCATCCTGCTCACAGGGCTGGTC
TTCCTCTTCTGCAGCCTGCCCCTCAGCATTCAGTTATTCCTATTATACTGGATCGAGAAGGATTTGCATG
ACTTACCTTGTGTTGTTCGTTTAATTTCCATTTTCCTGTCTGCTCTTAACAGCAGTGCCAACCCCATCAT
TTACTTCTTCATGGGCTCCTTTAGGCAGCTTCAAAACAGGAAGACCCTCAAGCTGGTTCTCCAGAGGGCT
CTGCAGGACATGCTTGAGGTGGATGAAGGTGGAGGGCAGCTTCCTGAGGAAACCCTGAAGCTGTCGGGAA
GCAGATTGGGGCCATGAGGAAGA

The disclosed NOV10 polypeptide (SEQ ID NO:31) encoded by SEQ ID NO:30 is 330 amino acid residues and is presented using the one-letter code in Table 10B. The disclosed NOV 10 protein was analyzed for signal peptide prediction and cellular localization. SignalP results predict that NOV10 is cleaved between position 65 and 66 of SEQ ID NO:31, i.e., at the slash in the amino acid sequence TFS-LY. Psort and Hydropathy profiles also predict that NOV10 contains a signal peptide and is localized in the plasma membrane (certainty=0.6000). It may also localize to the Golgi body.

TABLE 10B
Encoded NOV10 protein sequence (SEQ ID NO:31).

MNPTIPALDTEIAPISDTEETHPHRCGMEVLVLIVLILIIDLVGLAGNAVMLWLLGFCMHSNTFSLYILN
LARADFLCTCFQIITFINFFSDFVSSLSIHFSRFVTTVLFSACITGLSMLSTISTEHRLSVLWPIWYCCH
CPTHLSAVMCVLLWALSLLQSILEWMFCSFLFSDVDSDNWCQILDFLTAVWLIFLSVVLCGFTLVLLVRI
ICGSQKMPLTRLYVTILLTGLVFLFCSLPLSIQLFLLYWIEKDLDDLPCVVRLISIFLSALNSSANPIIY
FFMGSFRQLQNRKTLKLVLQRALQDMLEVDEGGGQLPEETLKLSGSRLGP

The NOV10 nucleic acids and proteins of the invention are useful in potential therapeutic applications implicated in used in the treatment of infections such as bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The polypeptides can be used as immunogens to produce antibodies specific for the invention, and as vaccines. They can also be used to screen for potential agonist and antagonist compounds. For example, a cDNA encoding the GPCR-like protein may be useful in gene therapy, and the GPCR-like protein may be useful when administered to a subject in need thereof. By way of nonlimiting example, the compositions of the present invention will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The novel nucleic acid encoding GPCR-like protein, and the GPCR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

In all BLAST alignments described herein, the “E-value” or “Expect” value is a numeric indication of the probability that the aligned sequences could have achieved their similarity to the BLAST query sequence by chance alone, within the database that was searched. All BLAST analyses described herein were performed using the NOV10 amino acid sequence.

A BLASTX search was performed against public protein databases. The amino acid sequence of the protein of the invention was found to have 89 of 265 amino acid residues (33%) identical to, 146 of 265 residues (54%) positive, and gaps at 10 of 265 residues, with the 324 amino acid putative protein from Mus musculus (gi|12853220|dbj|BAB29684.1| (AK015036)), E=6e-26). The disclosed protein is also similar to the proteins disclosed in Table 10C.

TABLE 10C
BLAST results for NOV10.

| Gene Index/ | Protein/ | Length | Identity | Positives | | |
|---|---|---|---|---|---|---|
| Identifier | Organism | (aa) | (%) | (%) | Expect | Gap |
| Gi\|12853220\|dbj\| | Putative | 324 | 89/265 | 146/265 | 6e-26 | 10/ |
| BAB29684.1\| | Mus musculus | | (33%) | (54%) | | 265 |
| (AK015036) | | | | | | |
| Gi\|6981186\|ref\| | MAS1 oncogene | 324 | 91/266 | 144/266 | 1e-25 | 12/ |
| NP_036889.1\| | Rattus norvegicus | | (34%) | (53%) | | 266 |
| Gi\|6678804\|ref\| | MAS1 oncogene | 324 | 85/264 | 141/264 | 5e-25 | 8/ |
| NP_032578.1\| | Mus musculus | | (32%) | (53%) | | 264 |
| Gi\|547920\|sp\| | MAS-RELATED G | 378 | 92/270 | 143/270 | 7e-25 | 27/ |
| P35410\|MRG_HUMAN | PROTEIN-COUPLED | | (34%) | (52%) | | 270 |
| | RECEPTOR MRG | | | | | |
| | Homo sapien | | | | | |
| Gi\|12711487\|gb\| | G-protein | 321 | 85/262 | 135/262 | 1e-24 | 9/ |
| AAK01865.1\| | coupled receptor | | (32%) | (51%) | | 262 |
| AF295365_1 | GPR90 | | | | | |
| (AG295365) | Mus musculus | | | | | |

A ClustalW analysis comparing disclosed proteins of the invention with related OR protein sequences (as determined by BLASTX analysis) is given in Table 10D, with NOV10 shown on line 1.

[image]

[image]

In the ClustalW alignment of the NOV10 protein, as well as all other ClustalW analyses described herein, the black outlined amino acid residues indicate regions of conserved sequence (i.e., regions that may be required to preserve structural or functional properties), whereas non-highlighted amino acid residues are less conserved and can potentially be mutated to a much broader extent without altering protein structure or function.

The presence of identifiable domains in NOV 10, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interior. DOMAIN results, e.g., for NOV9, were collected from the Conserved Domain Database (CDD) with Reverse Position Specific BLAST analyses. This BLAST analysis software samples domains found in the Smart and Pfam collections. For all successive DOMAIN sequence alignments, fully conserved single residues are indicated by black shading and “strong” semi-conserved residues are indicated by grey shading. The “strong” group of conserved amino acid residues may be any one of the following groups of amino acids: STA, NEQK, NHQK, NDEQ, QHRK, MILV, MILF, HY, FYW.

The region from amino acid residue 47 through 210 (SEQ ID NO:31) most probably (E=8e⁻⁸) contains a “seven transmembrane receptor (rhodopsin family) fragment” domain, aligned here with residues 1-166 of the 7tm_{—}1 entry (TM7, SEQ ID NO:39) of the Pfam database. This indicates that the NOV10 sequence has properties similar to those of other proteins known to contain this domain as well as to the 377 amino acid 7tm domain itself. NOV10 also has identity to another region of the TM7 protein. The region from amino acid residue 225 through 280 (of SEQ ID NO:31) aligns with amino acid residues 325-377 of TM7 (E=6e-04). Table 10E shows the results of the domain analysis of NOV10 and the TM7 protein.

[image]

[image]

The representative member of the 7 transmembrane receptor family is the D2 dopamine receptor from Bos taurus (SWISSPROT: locus D2DR_BOVIN, accession P20288; gene index 118205). The D2 receptor is an integral membrane protein and belongs to Family 1 of G-protein coupled receptors. The activity of the D2 receptor is mediated by G proteins, which inhibit adenylyl cyclase. Chio et al., Nature 343:255-269 (1990). Residues 51-427 of this 444 amino acid protein are considered to be the representative TM7 domain, shown in Table 10F.

TABLE 10F
Amino Acid sequence for TM7 (SEQ ID NO:39).

GNVLVCMAVSREKALQTTTNYLIVSLAVADLLVATLVMPWVVYLEVVGEWKFSRIHCDIF
VTLDVMMCTASILNLCAISIDRYTAVAMPMLYNTRYSSKRRVTVMIAIVWVLSFTISCPM
LFGLNNTDQNECIIANPAFVVYSSIVSFYVPFIVTLLVYIKIYIVLRRRRKRVNTKRSSR
AFRANLKAPLKGNCTHPEDMKLCTVIMKSNGSFPVNRRRVEAARRAQELEMEMLSSTSPP
ERTRYSPIPPSHHQLTLPDPSHHGLHSTPDSPAKPEKNGHAKTVNPKIAKIFEIQSMPNG
KTRTSLKTMSRRKLSQQKEKKATQMLAIVLGVFIICWLPFFITHILNIHCDCNIPPVLYS
AFTWLGYVNSAVNPIIY

The 7 transmembrane receptor family includes a number of different proteins, including, for example, serotonin receptors, dopamine receptors, histamine receptors, andrenergic receptors, cannabinoid receptors, angiotensin II receptors, chemokine receptors, opioid receptors, G-protein coupled receptor (GPCR) proteins, olfactory receptors (OR), and the like. Some proteins and the Protein Data Base Ids/gene indexes include, for example: rhodopsin (129209); 5-hydroxytryptamine receptors; (112821, 8488960, 112805, 231454, 1168221, 398971, 112806); G protein-coupled receptors (119130, 543823, 1730143, 132206, 137159, 6136153, 416926, 1169881, 136882, 134079); gustatory receptors (544463, 462208); c-x-c chemokine receptors (416718, 128999, 416802, 548703, 1352335); opsins (129193, 129197, 129203); and olfactory receptor-like proteins (129091, 1171893, 400672, 548417).

The nucleic acids and proteins of NOV10 are useful in potential therapeutic applications implicated in various NOV- or olfactory receptor (OR)-related pathologies and/or disorders. For example, a cDNA encoding the G-protein coupled receptor-like protein may be useful in gene therapy, and the G-protein coupled receptor-like protein may be useful when administered to a subject in need thereof. The novel nucleic acid encoding NOV10 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. The NOVX nucleic acids and proteins are useful in potential diagnostic and therapeutic applications implicated in various diseases and disorders described below and/or other pathologies. For example, the compositions of the present invention will have efficacy for treatment of patients suffering from: cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, fertility, hemophilia, hypercoagulation, idiopathic thrombocytopenic purpura, immunodeficiencies, graft versus host disease, bronchial asthma, and other diseases, disorders and conditions of the like. By way of nonlimiting example, the compositions of the present invention will have efficacy for treatment of patients suffering from neoplasm, adenocarcinoma, lymphoma, prostate cancer, uterus cancer, immune response, AIDS, asthma, Crohn's disease, multiple sclerosis, and Albright Hereditary Ostoeodystrophy. Additional NOV-related diseases and disorders are mentioned throughout the Specification.

Further, the protein similarity information, expression pattern, and map location for NOV10 suggests that NOV10 may have important structural and/or physiological functions characteristic of the NOV family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immuno-specifically to the novel NOV10 substances for use in therapeutic or diagnostic methods. These antibodies may be generated according to methods known in the art, using prediction from hydrophobicity charts, as described in the “Anti-NOVX Antibodies” section below.

A ClustalW alignment of NOV 4a, NOV4b, NOV5, NOV6, NOV7, NOV8, and NOV9 is shown in Table 11.

[image]

[image]

A summary of all of the NOVX nucleic acids and proteins of the invention is provided in Table 12.

TABLE 12
Summary Of Nucleic Acids And Proteins Of The Invention

| | | | Nucleic | Amino |
|---|---|---|---|---|
| | | | Acid | Acid |
| | | | SEQ ID | SEQ ID |
| Name | Tables | Clone; Description of Homolog | NO | NO |
| NOV1 | 1A, 1B, | NOV1a: dj408b20_B, olfactory receptor | 1 | 2 |
| | 1C, 1D, | NOV1b: dj408b20B-1, olfactory receptor | 3 | 4 |
| | 1E, 1F, | NOV1c: CG50369-01, olfactory receptor | 5 | 6 |
| | 1G, 1H, | | | |
| | 1I, 1J, 1K | | | |
| NOV2 | 2A, 2B, | NOV2a: 6-L-19D, olfactory receptor | 7 | 8 |
| | 2C, 2D, | NOV2b: 6_L_19_D_da1, olfactory receptor | 9 | 10 |
| | 2E, 2F, | NOV2c: 6_L_19_D_da2, olfactory receptor | 11 | 12 |
| | 2G, 2H, | NOV2d: 6_L_19_D_da1, olfactory receptor | 13 | 14 |
| | 2I, 2J, 2K, | | | |
| | 2L, 2M | | | |
| NOV3 | 3A, 3B, | NOV3: 6-L-19-E, olfactory receptor | 15 | 16 |
| | 3C, 3D, | | | |
| | 3E, 3F | | | |
| NOV4 | 4A, 4B, | NOV4a: 95-h-6-A, olfactory receptor | 17 | 18 |
| | 4C, 4D, | NOV4b: 95-h-6-A1, olfactory receptor | 19 | 18 |
| | 4E, 4F, | | | |
| | 4G, 4H, 4I | | | |
| NOV5 | 5A, 5B, | NOV5: 95-h-6-B, olfactory receptor | 20 | 21 |
| | 5C, 5D, | | | |
| | 5E, 5F | | | |
| NOV6 | 6A, 6B, | NOV6: 95-h-6-C, olfactory receptor | 22 | 23 |
| | 6C, 6D, | | | |
| | 6E, 6F | | | |
| NOV7 | 7A, 7B, | NOV7: 95-h-6-D, olfactory receptor | 24 | 25 |
| | 7C, 7D, | | | |
| | 7E, 7F | | | |
| NOV8 | 8A, 8B, | NOV8: 95-h-6-E, olfactory receptor | 26 | 27 |
| | 8C, 8D, | | | |
| | 8E, 8F | | | |
| NOV9 | 9A, 9B, | NOV9: 95-h-6-F, olfactory receptor | 28 | 29 |
| | 9C, 9D, | | | |
| | 9E, 9F | | | |
| NOV10 | 10A, 10B, | NOV10: AC023078_A, olfactory receptor | 30 | 31 |
| | 10C, 10D, | | | |
| | 10E, 10F | | | |

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step o5f post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence of SEQ ID NOS:1, 3, 5, 7, 9, 11, 13, 15, 17, 19, 20, 22, 24, 26, 28, and 30, or a complement of this aforementioned nucleotide sequence, can be isolated using standard molecular biology techniques and the sequence information provided herein. Using all or a portion of the nucleic acid sequence of SEQ ID NOS:1, 3, 5, 7, 9, 11, 13, 15, 17, 19, 20, 22, 24, 26, 28, and 30 as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NOS:1, 3, 5, 7, 9, 11, 13, 15, 17, 19, 20, 22, 24, 26, 28, and 30, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:1, 3, 5, 7, 9, 11, 13, 15, 17, 19, 20, 22, 24, 26, 28, and 30, or a portion of this nucleotide sequence (e.g. a fragment that can be used as a probe or primer or a fragment encoding a biologically-active portion of an NOVX polypeptide). A nucleic acid molecule that is complementary to the nucleotide sequence shown in SEQ ID NOS:1, 3, 5, 7, 9, 11, 13, 15, 17, 19, 20, 22, 24, 26, 28, and 30, is one that is sufficiently complementary to the nucleotide sequence shown in SEQ ID NOS:1, 3, 5, 7, 9, 11, 13, 15, 17, 19, 20, 22, 24, 26, 28, and 30, that it can hydrogen bond with little or no mismatches to the nucleotide sequence shown SEQ ID NOS:1, 3, 5, 7, 9, 11, 13, 15, 17, 19, 20, 22, 24, 26, 28, and 30, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS:1, 3, 5, 7, 9, 11, 13, 15, 17, 19, 20, 22, 24, 26, 28, and 30, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NOS:1, 3, 5, 7, 9, 11, 13, 15, 17, 19, 20, 22, 24, 26, 28, and 30; or an anti-sense strand nucleotide sequence of SEQ ID NOS:1, 3, 5, 7, 9, 11, 13, 15, 17, 19, 20, 22, 24, 26, 28, and 30; or of a naturally occurring mutant of SEQ ID NOS:1, 3, 5, 7, 9, 11, 13, 15, 17, 19, 20, 22, 24, 26, 28, and 30.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS:1, 3, 5, 7, 9, 11, 13, 15, 17, 19, 20, 22, 24, 26, 28, and 30 that encodes a polypeptides having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown SEQ ID NOS:1, 3, 5, 7, 9, 11, 13, 15, 17, 19, 20, 22, 24, 26, 28, and 30 due to degeneracy of the genetic code and thus encode the same NOVX proteins as that encoded by the nucleotide sequences shown in SEQ ID NOS:1, 3, 5, 7, 9, 11, 13, 15, 17, 19, 20, 22, 24, 26, 28, and 30. In another embodiment, an isolated nucleic acid molecule of the invention has a nucleotide sequence encoding a protein having an amino acid sequence shown in SEQ ID NOS:2, 4, 6, 8, 10, 12, 14, 16, 18, 21, 23, 25, 27, 29, and 31.

In addition to the human NOVX nucleotide sequences shown in SEQ ID NOS:1, 3, 5, 7, 9, 11, 13, 15, 17, 19, 20, 22, 24, 26, 28, and 30 it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human sequence SEQ ID NOS:1, 3, 5, 7, 9, 11, 13, 15, 17, 19, 20, 22, 24, 26, 28, and 30 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:1, 3, 7, 9, 11, 13, 15, 17, 19, 20, 22, 24, 26, 28, and 30. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6X SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2X SSC, 0.01% BSA at 50° C. An isolated nucleic acid molecule of the invention that hybridize under stringent conditions to the sequence of SEQ ID NOS:1, 3, 5, 7, 9, 11, 13, 15, 17, 19, 20, 22, 24, 26, 28, and 30 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:1, 3, 5, 7, 9, 11, 13, 15, 17, 19, 20, 22, 24, 26, 28, and 30 or fragments, analogs or derivatives thereof, under conditions of moderate stringency is provided. A non-limiting example of moderate stringency hybridization conditions are hybridization in 6× SSC, 5× Denhardt's solution, 0.5% SDS and 100 mg/ml denatured salmon sperm DNA at 55° C., followed by one or more washes in 1× SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NOS:1, 3, 5, 7, 9, 11, 13, 15, 17, 19, 20, 22, 24, 26, 28, and 30 or fragments, analogs or derivatives thereof, under conditions of low stringency, is provided. A non-limiting example of low stringency hybridization conditions are hybridization in 35% formamide, 5× SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2× SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences of SEQ ID NOS:1, 3, 5, 7, 9, 11, 13, 15, 17, 19, 20, 22, 24, 26, 28, and 30 thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence of SEQ ID NOS:2, 4, 6, 8, 10, 12, 14, 16, 18, 21, 23, 25, 27, 29, and 31. A “inon-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS:2, 4, 6, 8, 10, 12, 14, 16, 18, 21, 23, 25, 27, 29, and 31 yet retain biological activity. In one embodiment, the isolated nucleic acid molecule comprises a nucleotide sequence encoding a protein, wherein the protein comprises an amino acid sequence at least about 45% homologous to the amino acid sequences of SEQ ID NOS:2, 4, 6, 8, 10, 12, 14, 16, 18, 21, 23, 25, 27, 29, and 31. Preferably, the protein encoded by the nucleic acid molecule is at least about 60% homologous to SEQ ID NOS:2, 4, 6, 8, 10, 12, 14, 16, 18, 21, 23, 25, 27, 29, and 31; more preferably at least about 70% homologous to SEQ ID NOS:2, 4, 6, 8, 10, 12, 14, 16, 18, 21, 23, 25, 27, 29, and 31; still more preferably at least about 80% homologous to SEQ ID NOS:2, 4, 6, 8, 10, 12, 14, 16, 18, 21, 23, 25, 27, 29, and 31; even more preferably at least about 90% homologous to SEQ ID NOS:2, 4, 6, 8, 10, 12, 14, 16, 18, 21, 23, 25, 27, 29, and 31; and most preferably at least about 95% homologous to SEQ ID NOS:2, 4, 6, 8, 10, 12, 14, 16, 18, 21, 23, 25, 27, 29, and 31.

An isolated nucleic acid molecule encoding an NOVX protein homologous to the protein of SEQ ID NOS:2, 4, 6, 8, 10, 12, 14, 16, 18, 21, 23, 25, 27, 29, and 31 can be created by introducing one or more nucleotide substitutions, additions or deletions into the nucleotide sequence of SEQ ID NOS:1, 3, 5, 7, 9, 11, 13, 15, 17, 19, 20, 22, 24, 26, 28, and 30 such that one or more amino acid substitutions, additions or deletions are introduced into the encoded protein.

Mutations can be introduced into SEQ ID NOS:2, 4, 6, 8, 10, 12, 14, 16, 18, 21, 23, 25, 27, 29, and 31 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis of SEQ ID NOS:1, 3, 5, 7, 9, 11, 13, 15, 17, 19, 20, 22, 24, 26, 28, and 30, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS:1, 3, 5, 7, 9, 11, 13, 15, 17, 19, 20, 22, 24, 26, 28, and 30, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS:2, 4, 6, 8, 10, 12, 14, 16, 18, 21, 23, 25, 27, 29, and 31, or antisense nucleic acids complementary to an NOVX nucleic acid sequence of SEQ ID NOS:1, 3, 5, 7, 9, 11, 13, 15, 17, 19, 20, 22, 24, 26, 28, and 30, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (see, e.g., Inoue, et al. 1987. Nucl. Acids Res. 15: 6131-6148) or a chimeric RNA-DNA analogue (see, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS:1, 3, 5, 7, 9, 11, 13, 15, 17, 19, 20, 22, 24, 26, 28, and 30). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (see, Hyrup, et al., 1996.supra); or as probes or primers for DNA sequence and hybridization (see, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS:2, 4, 6, 8, 10, 12, 14, 16, 18, 21, 23, 25, 27, 29, and 31. The invention also includes a mutant or variant protein any of whose residues may be changed from the corresponding residues shown in SEQ ID NOS:2, 4, 6, 8, 10, 12, 14, 16, 18, 21, 23, 25, 27, 29, and 31 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS:2, 4, 6, 8, 10, 12, 14, 16, 18, 21, 23, 25, 27, 29, and 31) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown in SEQ ID NOS:2, 4, 6, 8, 10, 12, 14, 16, 18, 21, 23, 25, 27, 29, and 31. In other embodiments, the NOVX protein is substantially homologous to SEQ ID NOS:2, 4, 6, 8, 10, 12, 14, 16, 18, 21, 23, 25, 27, 29, and 31, and retains the functional activity of the protein of SEQ ID NOS:2, 4, 6, 8, 10, 12, 14, 16, 18, 21, 23, 25, 27, 29, and 31, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS:2, 4, 6, 8, 10, 12, 14, 16, 18, 21, 23, 25, 27, 29, and 31, and retains the functional activity of the NOVX proteins of SEQ ID NOS:2, 4, 6, 8, 10, 12, 14, 16, 18, 21, 23, 25, 27, 29, and 31.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS:1, 3, 5, 7, 9, 11, 13, 15, 17, 19, 20, 22, 24, 26, 28, and 30.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein (SEQ ID NOS:2, 4, 6, 8, 10, 12, 14, 16, 18, 21, 23, 25, 27, 29, and 31), whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediate signal transduction in vivo. The NOVX immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, A 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g, Arkin and Yourvan, 1992. Proc. Natl. Acad. Sci. USA 89: 7811-7815; Delgrave, et al., 1993. Protein Engineering 6:327-331.

Anti-NOVX Antibodies

The invention encompasses antibodies and antibody fragments, such as F_{ab} or (F_{ab})₂, that bind immunospecifically to any of the NOVX polypeptides of said invention.

An isolated NOVX protein, or a portion or fragment thereof, can be used as an immunogen to generate antibodies that bind to NOVX polypeptides using standard techniques for polyclonal and monoclonal antibody preparation. The full-length NOVX proteins can be used or, alternatively, the invention provides antigenic peptide fragments of NOVX proteins for use as immunogens. The antigenic NOVX peptides comprises at least 4 amino acid residues of the amino acid sequence shown in SEQ ID NOS:2, 4, 6, 8, 10, 12, 14, 16, 18, 21, 23, 25, 27, 29, and 31 and encompasses an epitope of NOVX such that an antibody raised against the peptide forms a specific immune complex with NOVX. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of NOVX that is located on the surface of the protein (e.g., a hydrophilic region). As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation (see, e.g., Hopp and Woods, 1981. Proc. Nat. Acad. Sci. USA 78: 3824-3828; Kyte and Doolittle, 1982. J. Mol. Biol. 157: 105-142, each incorporated herein by reference in their entirety).

As disclosed herein NOVX protein sequences of SEQ ID NOS:2, 4, 6, 8, 10, 12, 14, 16, 18, 21, 23, 25, 27, 29, and 31, or derivatives, fragments, analogs or homologs thereof, may be utilized as immunogens in the generation of antibodies that immunospecifically-bind these protein components. The term “antibody” as used herein refers to immunoglobulin molecules and immunologically-active portions of immunoglobulin molecules, i.e., molecules that contain an antigen binding site that specifically-binds (immunoreacts with) an antigen, such as NOVX. Such antibodies include, but are not limited to, polyclonal, monoclonal, chimeric, single chain, F_{ab} and F_{(ab′)2} fragments, and an F_{ab} expression library. In a specific embodiment, antibodies to human NOVX proteins are disclosed. Various procedures known within the art may be used for the production of polyclonal or monoclonal antibodies to an NOVX protein sequence of SEQ ID NOS:2, 4, 6, 8, 10, 12, 14, 16, 18, 21, 23, 25, 27, 29, and 31, or a derivative, fragment, analog or homolog thereof. Some of these proteins are discussed below.

For the production of polyclonal antibodies, various suitable host animals (e.g., rabbit, a goat, mouse or other mammal) may be immunized by injection with the native protein, or a synthetic variant thereof, or a derivative of the foregoing. An appropriate immunogenic preparation can contain, for example, recombinantly-expressed NOVX protein or a chemically-synthesized NOVX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against NOVX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of NOVX. A monoclonal antibody composition thus typically displays a single binding affinity for a particular NOVX protein with which it immunoreacts. For preparation of monoclonal antibodies directed towards a particular NOVX protein, or derivatives, fragments, analogs or homologs thereof, any technique that provides for the production of antibody molecules by continuous cell line culture may be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see e.g., Cole, et al., 1985. In: MONOCLONAL ANTIBODIES AND CANCER THERAPY, Alan R. Liss, Inc., pp. 77-96). Human monoclonal antibodies may be utilized in the practice of the invention and may be produced by using human hybridomas (see, e.g., Cote, et al., 1983. Proc Natl Acad Sci USA 80: 2026-2030) or by transforming human B-cells with Epstein Barr Virus in vitro (see, e.g., Cole, et al., 1985. In: MONOCLONAL ANTIBODIES AND CANCER THERAPY, Alan R. Liss, Inc., pp. 77-96). Each of the above citations is incorporated herein by reference in their entirety.

According to the invention, techniques can be adapted for the production of single-chain antibodies specific to an NOVX protein (see, e.g., U.S. Pat. No. 4,946,778). In addition, methods can be adapted for the construction of Fab expression libraries (see, e.g., Huse, et al., 1989. Science 246: 1275-1281) to allow rapid and effective identification of monoclonal F_{ab} fragments with the desired specificity for an NOVX protein or derivatives, fragments, analogs or homologs thereof. Non-human antibodies can be “humanized” by techniques well known in the art. See, e.g., U.S. Pat. No. 5,225,539. Antibody fragments that contain the idiotypes to an NOVX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent; and (iv) Fᵥ fragments.

Additionally, recombinant anti-NOVX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. Nos. 4,816,567; 5,225,539; European Patent Application No. 125,023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314 :446-449; Shaw, et al., 1988. J. Natl. Cancer Inst. 80: 1553-1559); Morrison(1985) Science 229:1202-1207; Oi, et al. (1986) BioTechniques 4:214; Jones, et al., 1986. Nature 321: 552-525; Verhoeyan, et al., 1988. Science 239: 1534; and Beidler, et al., 1988. J. Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells its and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are a encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONNG: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the impression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences of SEQ ID NOS:1, 3, 5, 7, 9, 11, 13, 15, 17, 19, 20, 22, 24, 26, 28, and 30 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS:1, 3, 5, 7, 9, 11, 13, 15, 17, 19, 20, 22, 24, 26, 28,and 30), but more preferably, is a non-human homologue of a human NOVX gene. For example, a mouse homologue of human NOVX gene of SEQ ID NOS:1, 3, 5, 7, 9, 11, 13, 15, 17, 19, 20, 22, 24, 26, 28, and 30 can be used to construct a homologous recombination vector suitable for altering an endogenous NOVX gene in the mouse genome. In one embodiment, the vector is designed such that, upon homolog recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction “double” transgenic animals, e.g., by g two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and No Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12:145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 9 422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comp rises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Tritono X-100, Triton® X-114, Thesit®, Isotridecypoly(ethylene glycol ether),, N-dodecyl—N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus ate gene regions associated with gendisease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS:1, 3, 5, 7, 9, 11, 13, 15, 17, 19, 20, 22, 24, 26, 28, and 30, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:1, 3, 5, 7, 9, 11, 13, 15, 17, 19, 20, 22, 24, 26, 28, and 30 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS:1, 3, 5, 7, 9, 11, 13, 15, 17, 19, 20, 22, 24, 26, 28, and 30, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., F_{ab} or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qu Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38:147-159).

Other methods for detecting mutations in the NOVX gene include methods in which protection from cleavage agents is used to detect mismatched bases in RNA/RNA or RNA/DNA heteroduplexes. See, e.g. Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxlylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-29>5. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. Set, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7:5.

In yet another embodiment, the movement of mutant or wild-type fragments in polyacrylamide gels containing a gradient of denaturant is assayed using denaturing gradient gel electrophoresis (DGGE). See, e.g., Myers, et al, 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA. or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6:1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens., Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (AT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical Trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endoggenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or ,m agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious diseases, anorexia, cancer-associated cancer, neurodegeneritive disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies which immunospecifically-bind to the novel substances of the invention for use in therapeutic or diagnostic methods.

### EQUIVALENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS:2, 4, 6, 8, 10, 12, 14, 16, 18, 21, 23, 25, 27, 29, and 31;
(b) a variant of a mature form of an amino acid sequence selected from the group consisting of SEQ ID NOS:2, 4, 6, 8, 10, 12, 14, 16, 18, 21, 23, 25, 27, 29, and 31, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 21, 23, 25, 27, 29, and 31; and
(d) a variant of an amino acid sequence selected from the group consisting of SEQ ID NOS:2, 4, 6, 8, 10, 12, 14, 16, 18, 21, 23, 25, 27, 29, and 31, wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting of SEQ ID NOS:2, 4, 6, 8, 10, 12, 14, 16, 18, 21, 23, 25, 27, 29, and 31.

3. The polypeptide of claim 2, wherein said allelic variant comprises an amino acid sequence that is, the translation of a nucleic acid sequence differing by a single nucleotide from a nucleic acid sequence selected from the group consisting of SEQ ID NOS:1, 3, 5, 7, 9, 11, 13, 15, 17, 19, 20, 22, 24, 26, 28, and 30.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS:2, 4, 6, 8, 10, 12, 14, 16, 18, 21, 23, 25, 27, 29, and 31;
(b) a variant of a mature form of an amino acid sequence selected from the group consisting of SEQ ID NOS:2, 4, 6, 8, 10, 12, 14, 16, 18, 21, 23, 25, 27, 29, and 31, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 21, 23, 25, 27, 29, and 31;
(d) a variant of an amino acid sequence selected from the group consisting SEQ ID NOS:2, 4, 6, 8, 10, 12, 14, 16, 18, 21, 23, 25, 27, 29, and 31, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2, 4, 6, 8, 10, 12, 14, 16, 18, 21, 23, 25, 27, 29, and 31, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:1, 3, 5, 7, 9, 11, 13, 15, 17, 19, 20, 22, 24, 26, 28, and 30.

9. The nucleic acid molecule of claim 5, wherein said nucleic acid molecule comprises a nucleotide sequence selected from the group consisting of:
(a) a nucleotide sequence selected from the group consisting of SEQ ID NOS:1, 3, 5, 7, 9, 11, 13, 15, 17, 19, 20, 22, 24, 26, 28, and 30;
(b) a nucleotide sequence differing by one or more nucleotides from a nucleotide sequence selected from the group consisting of SEQ ID NOS:1, 3, 5, 7, 9, 11, 13, 15, 17, 19, 20, 22, 24, 26, 28, and 30, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting of SEQ ID NOS:1, 3, 5, 7, 9, 11, 13, 15, 17, 19, 20, 22, 24, 26, 28, and 30, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide,
thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration i a expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

27. The method of claim 26 wherein the disorder is related to cell signal processing and metabolic pathway modulation.

28. The method of claim 26, wherein said subject is a human.

29. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

30. The method of claim 29 wherein the disorder is related to cell signal processing and metabolic pathway modulation.

31. The method of claim 29, wherein said subject is a human.

32. A method of treating or preventing a NOVX-associated disorder, said method comprising administering to a subject in which such treatment or prevention is desired the antibody of claim 15 in an amount sufficient to treat or prevent said NOVX-associated disorder in said subject.

33. The method of claim 32 wherein the disorder is diabetes.

34. The method of claim 32 wherein the disorder is related to cell signal processing and metabolic pathway modulation.

35. The method of claim 32, wherein the subject is a human.

36. A pharmaceutical composition comprising the polypeptide of claim 1 and a pharmaceutically-acceptable carrier.

37. A pharmaceutical composition comprising the nucleic acid molecule of claim 5 and a pharmaceutically-acceptable carrier.

38. A pharmaceutical composition comprising the antibody of claim 15 and a pharmaceutically-acceptable carrier.

39. A kit comprising in one or more containers, the pharmaceutical composition of claim 36.

40. A kit comprising in one or more containers, the pharmaceutical composition of claim 37.

41. A kit comprising in one or more containers, the pharmaceutical composition of claim 38.

42. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

43. The method of claim 42 wherein the predisposition is to cancers.

44. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

45. The method of claim 44 wherein the predisposition is to a cancer.

46. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS:2, 4, 6, 8, 10, 12, 14, 16, 18, 21, 23, 25, 27, 29, and 31, or a biologically active fragment thereof.

47. A method of treating a pathological state in a mammal, the method comprising administering to the mammal the antibody of claim 15 in an amount sufficient to alleviate the pathological state.

48. A method for the screening of a candidate substance interacting with an olfactory receptor polypeptide selected from the group consisting of SEQ ID NOS:2, 4, 6, 8, 10, 12, 14, 16, 18, 21, 23, 25, 27, 29, and 31, or fragments or variants thereof, comprises the following steps:
a) providing a polypeptide selected from the group consisting of the sequences of SEQ ID NOS:2, 4, 6, 8, 10, 12, 14, 16, 18, 21, 23, 25, 27, 29, and 31, or a peptide fragment or a variant thereof;
b) obtaining a candidate substance;
c) contacting said polypeptide with said candidate substance; and
d) detecting the complexes formed, if any, between said polypeptide and said candidate substance.

49. A method for the screening of ligand molecules interacting with an olfactory receptor polypeptide selected from the group consisting of SEQ ID NOS:2, 4, 6, 8, 10, 12, 14, 16, 18, 21, 23, 25, 27, 29, and 31, wherein said method comprises:
a) providing a recombinant eukaryotic host cell containing a nucleic acid encoding a polypeptide selected from the group consisting of the polypeptides comprising the amino acid sequences SEQ ID NOS:2, 4, 6, 8, 10, 12, 14, 16, 18, 21, 23, 25, 27, 29, and 31;
b) preparing membrane extracts of said recombinant eukaryotic host cell;
c) contacting the membrane extracts prepared at step b) with a selected ligand molecule; and
d) detecting the production level of second messengers metabolites.

50. A method for the screening of ligand molecules interacting with an olfactory receptor polypeptide selected from the group consisting of SEQ ID NOS:2, 4, 6, 8, 10, 12, 14, 16, 18, 21, 23, 25, 27, 29, and 31, wherein said method comprises:
a) providing an adenovirus containing a nucleic acid encoding a polypeptide selected from the group consisting of polypeptides comprising the amino acid sequences SEQ ID NOS:2, 4, 6, 8, 10, 12, 14, 16, 18, 21, 23, 25, 27, 29, and 31;
b) infecting an olfactory epithelium with said adenovirus;
c) contacting the olfactory epithelium b) with a selected ligand molecule; and
d) detecting the increase of the response to said ligand molecule, if present.
